(19) United States
(12) Patent Application Publication
(10) Pub. No.: US 2024/0382447 A1
(43) Pub. Date: Nov. 21, 2024

(54) BENIGN PROSTATIC HYPERPLASIA TREATMENT SYSTEM

(71) Applicant: RESURGE THERAPEUTICS, INC., San Jose, CA (US)

(72) Inventors: Olof Mikael TROLLSAS, San Jose, CA (US); John J. STANKUS, Santra Cruz, CA (US); Shahram Shawn GHOLAMI, Monte Sereno, CA (US)

(21) Appl. No.: 18/786,236

(22) Filed: Jul. 26, 2024

Related U.S. Application Data
(63) Continuation-in-part of application No. 18/528,691, filed on Dec. 4, 2023, now Pat. No. 12,090,136.
(63) Continuation of application No. PCT/US2023/011776, filed on Jan. 27, 2023.
Application No. 18/528,691 is a continuation of application No. 18/062,436, filed on Dec. 6, 2022, now Pat. No. 11,957,654.
Application No. 18/062,436 is a continuation of application No. 17/727,675, filed on Apr. 22, 2022, now Pat. No. 11,602,516.
Application No. 17/727,675 is a continuation-in-part of application No. 17/727,675, filed on Apr. 22, 2022, now Pat. No. 11,602,516.
Application No. 17/727,675 is a continuation-in-part of application No. 17/727,680, filed on Apr. 22, 2022, now Pat. No. 11,974,979.
(60) Provisional application No. 63/304,599, filed on Jan. 29, 2022.
(60) Provisional application No. 63/304,599, filed on Jan. 29, 2022.

(51) Int. Cl. A61K 31/337; A61K 45/06; A61P 35/00

(52) U.S. Cl. CPC A61K 31/337; A61K 45/06; A61P 35/00

(57) ABSTRACT

Minimally invasive treatment methods for benign prostatic hyperplasia (BPH) tissue. A system includes a sustained release formulation comprising a cytostatic or cytotoxic drug, and an applicator or delivery system for local delivery of a composition comprising or consisting essentially of the sustained release formulation to the prostate. The applicator containing the composition is characterized by a KIR value of between 15 and 1,000 Centipoise per unit area.

### CROSS-REFERENCE TO RELATED APPLICATIONS

This application is a continuation-in-part of co-pending U.S. patent application Ser. No. 18/528,691 filed Dec. 4, 2023, which is a continuation of U.S. patent application Ser. No. 18/062,436 filed Dec. 6, 2022, (now U.S. Pat. No. 11,957,654, issued Apr. 16, 2024), which is a continuation of U.S. patent application Ser. No. 17/727,675 filed Apr. 22, 2022, (now U.S. Pat. No. 11,602,516, issued Mar. 14, 2023), which claims the benefit of U.S. Provisional Patent Application No. 63/304,599 filed Jan. 29, 2022. This application is also a continuation of PCT International Application No. PCT/US2023/011776 filed Jan. 27, 2023, which claims priority to U.S. Provisional Patent Application No. 63/304,599 filed Jan. 29, 2022, U.S. patent application Ser. No. 17/727,675 filed Apr. 22, 2022, (now U.S. Pat. No. 11,602,516, issued Mar. 14, 2023), and U.S. patent application Ser. No. 17/727,680 filed Apr. 22, 2022, (now U.S. Pat. No. 11,974,979, issued May 7, 2024). Each application is incorporated by reference in its entirety for all purposes.

### FIELD

Minimally invasive, local treatments for men's health and, more particularly, lower urinary tract symptoms.

### BACKGROUND

Benign Prostatic Hyperplasia (BPH) is a noncancerous increase in size of the prostate gland due to proliferation of glandular epithelial tissue, smooth muscle and connective tissue within the prostate transition zone that causes lower urinary tract symptoms. Lower urinary tract symptoms (LUTS) include voiding or obstructive symptoms such as hesitancy, poor and/or intermittent stream, straining, feeling of incomplete bladder emptying, and storage or irritative symptoms such as frequency, urgency, urge incontinence, and nocturia. It affects approximately half of men aged 50 and over and by age 80, 90% of men are affected. Treatment options consist of lifestyle changes, medications, various procedures, and surgery. Lifestyle changes consist of weight loss, exercise, and decreased caffeine consumption. With more significant symptoms, oral medications such as alpha blockers (e.g., terazosin) or 5-alpha-reductase inhibitors (e.g., finasteride) are prescribed. These medications, requiring daily dosing for patient compliance, may require a long onset to show efficacy, if at all, and carry side effects such as ejaculation changes, erectile dysfunction, weakness, headaches, and decreased libido.

There is an unmet clinical need to treat BPH with improved and sustained efficacy, administered via a less invasive procedure and with less associated side effects.

### BRIEF SUMMARY OF THE INVENTION

In one embodiment, set forth herein is a method for treating Benign Prostatic Hyperplasia (BPH) in a subject in need thereof, comprising: using a needle syringe containing a composition comprising a cytotoxic or cytostatic drug, a glycolide-based bioabsorbable copolymer, and a water soluble solvent capable of dissolving the drug and copolymer; and dispensing a total volume of the composition into a prostate to produce an efficacious outcome for treating BPH, including dispensing no more than a unit volume of the composition into a prostate at a first prostate location using the needle syringe.

In a second embodiment, set forth herein is a method for treating Benign Prostatic Hyperplasia (BPH) in a subject in need thereof, comprising: using a needle syringe containing a composition comprising a cytotoxic or cytostatic drug, a glycolide-based bioabsorbable copolymer, and a water soluble solvent capable of dissolving the drug and copolymer; and dispensing a total volume of the composition into a prostate to produce an efficacious outcome for treating BPH, including dispensing multiple unit volume of the composition into a prostate at a first prostate location using the needle syringe.

In a third embodiment, set forth herein is a method of treating Benign Prostatic Hyperplasia (BPH) in a subject in need thereof, comprising using an apparatus comprising a needle syringe containing a composition comprising a cytotoxic or cytostatic drug, a glycolide-based bioabsorbable copolymer, and a water soluble solvent capable of dissolving the drug and copolymer, wherein the composition has an absolute viscosity, p, and the needle syringe has a needle length L with inner diameter D; and dispensing a plurality of unit volumes, v, of the composition at a respective plurality of different locations in the prostate using the needle syringe; wherein a unit volume is from 0.05 ml to 0.2 ml; wherein the plurality of unit volumes is equal to a total volume for treating BPH; and wherein the apparatus has a KIR of between 10 to 300 or 40 to 400 and KIR is defined as

$$KIR = \frac{μ {L}^{2}}{vD} ( 1 {0}^{- 6} ) .$$

In a fourth embodiment, set forth herein is an apparatus for treating a prostate volume, PV, comprising: a composition comprising an SRF having an absolute viscosity, p, and in an amount of at least one unit volume, v; and a syringe containing the composition in a barrel of the syringe, the syringe including a needle having a length, L, and lumen diameter, D, and adapted for being placed in fluid communication with the composition; wherein the syringe holds at least n of the unit volumes, v, n is related to PV as n=PV/(200*v) and the n unit volumes equal a total volume for treating BPH; and wherein the apparatus has a KIR between 10 and 1000, 10 and 300, or 40 and 400, wherein the KIR is defined as (μ)*(L)\hat{ }/(vD)*10\hat{ }(−6).

In a fifth embodiment, set forth herein is an apparatus for treating a prostate volume, PV, comprising a composition comprising an SRF having an absolute viscosity, p, and in an amount of at least one unit volume, v; a syringe containing the composition in a barrel of the syringe, the syringe including a needle having a length, L, and lumen diameter, D, and adapted for being placed in fluid communication with the composition; and wherein the syringe holds at least n of the unit volumes and n is related to PV as n=PV/(200*v); and wherein L is between 15 and 20 cm, the barrel has a slenderness ratio of about 10 and 30, the needle has an inner diameter of 0.06 cm to about 0.08 cm, v is between 0.05 ml and 0.1 ml, and p is between 1065 cP and 4000 cP; and wherein the apparatus has a KIR between 10 and 1000, defined as (μ)*(L)\hat{ }/(vD)*10\hat{ }(−6).

In a sixth embodiment, set forth herein is a guide for providing incremental dosages from a syringe coupled to the guide, the syringe including a barrel, a finger rest extending from the barrel, and a plunger that is movable relative to the barrel, the guide including: (1) a main body extending along a central axis between a first end and a second end, the main body defining an interior space, the main body including: (a) an exterior wall; (b) an interior wall disposed radially inward of the exterior wall; and (c) a plurality of detents extending from the interior wall into the interior space, the plurality of detents being spaced apart from one another along the central axis of the main body; (2) a finger rest retaining portion at the second end of the main body, the finger rest retaining portion being configured to engage the finger rest of the syringe to prevent movement of the barrel relative to the main body; wherein, when the syringe is provided in the guide, the plunger extends into the interior space of the main body, and wherein, when the plunger is moved relative to the barrel, a first detent of the plurality of detents is configured to engage the plunger to resist movement of the plunger relative to the barrel, the plunger being movable past a first detent of the plurality of detents to a second detent of the plurality of detents.

In a seventh embodiment, set forth herein is a delivery device configured to mix a first composition with a second composition, the delivery device including: a barrel extending between a first end and a second end, the first end of the barrel being adapted for attachment to a needle; a septum disposed within the barrel, the septum dividing the barrel into a first chamber and a second chamber, the first chamber receiving the first composition, the second chamber receiving the second composition; and a plunger extending from the second end of the barrel, the plunger being movable relative to the barrel, the plunger being configured to break the septum to allow the first composition to mix with the second composition to form a mixture and force the mixture from the first end of the barrel into the needle. In some embodiments, the mixture could be mixed back and forth in the barrel more than once. In some embodiments, the mixture could be mixed using a distal static mixture attachment distal to the syringe yet proximal to the needle.

In an eighth embodiment, set forth herein is a kit including: at least two or more pre-filled syringes; wherein one of the at least two or more pre-filled syringes includes a composition including a cytotoxic drug powder or cytostatic drug powder, wherein one of the at least two or more pre-filled syringes includes a composition including a glycolide-based bioabsorbable copolymer and a water soluble solvent; and wherein the kit also includes instructions for mixing the pre-filled syringes.

In a ninth embodiment, set forth herein is a kit comprising: at least one syringe comprising a composition comprising a cytotoxic drug or cytostatic drug powder, a composition comprising a glycolide-based bioabsorbable copolymer and a water soluble solvent; and instructions for administering the composition.

In an tenth embodiment, set forth herein is a method for treating Benign Prostatic Hyperplasia (BPH) in a subject in need thereof, including: providing a guide set forth herein, a device set forth herein, a kit set forth herein, or a combination thereof, and dispensing a total volume of the composition into a prostate to produce an efficacious outcome for treating BPH, including dispensing no more than a unit volume of the composition into a prostate at a first prostate location using the needle syringe.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a PRIOR ART perspective view of a needle syringe.

FIG. 2A is a schematic side-view illustration of prostate-type needle syringes for delivering a composition that includes a sustained release formulation (SRF).

FIG. 2B is a schematic side-view illustration of prostate-type needle syringes for delivering a composition including a sustained release formulation (SRF).

FIG. 3A provides examples of K-Injectate Ratings (KIRs) for transrectal and perineal BPH treatments.

FIG. 3B provides examples of K-Injectate Ratings (KIRs) for transrectal and perineal BPH treatments.

FIG. 3C shows Drug Release Curves for SRFs A, B and C.

FIG. 4 shows a histopathology H&E staining microscope image of a sectioned canine prostate lobe 30 days after SRF1 treatment in BPH animal #21C0042. Outline shows a necrotic core and the extent of the drug effect around the 100 μL volume of SRF injectate (arrow). Scale bar equals 1 mm.

FIG. 5 shows histopathology H&E staining microscope image of a sectioned canine prostate lobe 90 days after SRF1 treatment in BPH animal #21C0046. Outline shows the necrotic core and the extent of drug effect around the 50 μL volume of SRF injectate (arrow). Scale bar equals 1 mm.

FIG. 6 shows histopathology H&E staining microscope image of a sectioned canine prostate lobe 90 days after SRF1 treatment in BPH animal #21C0050. Outline shows necrotic core and extent of drug effect around the 50 μL of volume of SRF injectate (arrow). Scale bar equals 1 mm.

FIG. 7 shows systemic drug concentrations measured by liquid chromatography mass spectroscopy (LCMS) as a function of time post of a SRF1 injection per animal.

FIG. 8 shows systemic drug mean concentrations measured by LCMS as a function of time post SRF1 injection per animal as a function of drug dosage. N=6 for 0 days to 30 days and N=3 60 days and 90 days, 4 animals with 5 mg and 2 animals with the 10 mg initiation.

FIG. 9 shows locations for injection of SRF1 in a prostate. FIG. 9 shows the locations in TABLE 7 for collecting prostate samples for pharmacokinetic (“pk”) tissue analysis. Circles indicate treatment samples (“tx”), rectangles indicate either adjacent reference samples (“adj”) or distal reference samples (“distal”). Numbers in diagram are the locations corresponding to TABLE 7.

FIG. 10A shows a transurethral-type needle syringe for delivering a composition including a sustained release formulation (SRF).

FIG. 10B shows a syringe having a depth limiter for not exceeding a predetermined depth of needle injection, and for dispensing a pre-determined unit volume of a composition including a sustained release formulation (SRF) into a prostate.

FIG. 10C show an example of a transurethral needle with depth markings.

FIG. 10D show an example of transurethral needle with depth markings.

FIG. 11A shows male and female syringes for point of procedure mixing of SRF drug (labelled Syringe B: Drug Powder) with solvent and polymer solution (labelled Syringe A: Polymer and Solvent Solution). FIG. 11A also shows the male and female syringes connected for mixing (labeled Mixing). FIG. 11A also shows a transrectal 20 G×2 cm needle with a syringe and SRF composition therein formulated and labeled as a Product Ready for Injection via a transrectal 20 G×2 cm needle.

FIG. 11B shows a self-contained multi-chamber syringe delivery device containing in a first chamber a drug powder and in a second chamber a water-soluble solvent and a polymer solution. Also shown is a transrectal needle with a protective cover.

FIG. 12 shows a point of care kit for treating BPH. FIG. 12 shows a desiccant and drug product polyethylene terephthalate glycol (PETG) packaging tray.

FIG. 13 shows a cross-section of the prostate with transurethral needle injection through the urethral wall.

FIG. 14A provides examples of K-Injectate Ratings (KIRs) for transurethral BPH treatments.

FIG. 14B provides examples of K-Injectate Ratings (KIRs) for transurethral BPH treatments.

FIG. 15 shows an embodiment of a guide for providing unit volume dosages from a syringe.

FIG. 16 shows an embodiment of a guide for providing unit volume dosages from a syringe.

FIG. 17 shows an embodiment of a guide for providing unit volume dosages from a syringe.

FIG. 18 shows an embodiment of a guide for providing unit volume dosages from a syringe.

FIG. 19 shows an embodiment of a guide for providing unit volume dosages from a syringe.

FIG. 20 shows an embodiment of a guide for providing unit volume dosages from a syringe.

FIG. 21 shows an embodiment of a guide for providing unit volume dosages from a syringe.

FIG. 22 shows an embodiment of a guide for providing unit volume dosages from a syringe.

FIG. 23 shows an embodiment of a guide for providing unit volume dosages from a syringe.

FIG. 24 shows an embodiment of a guide for providing unit volume dosages from a syringe.

FIG. 25 shows an embodiment of a guide for providing unit volume dosages from a syringe.

FIG. 26 shows an embodiment of a guide for providing unit volume dosages from a syringe.

FIG. 27 shows an embodiment of a guide for providing unit volume dosages from a syringe.

FIG. 28 shows an embodiment of a guide for providing unit volume dosages from a syringe.

FIG. 29 shows a self-contained syringe containing in a first chamber a drug powder, and in a second chamber a water-soluble solvent and a polymer solution.

FIG. 30 shows an embodiment of a guide for providing unit volume dosages from a syringe.

### DETAILED DESCRIPTION

The disclosure is generally directed to achieving a local delivery of a sustained release formulation at a specification for efficacious treatment of a target tissue associated with the prostate, and/or providing relief of urinary tract symptoms originating from or associated with an enlarged prostate while mitigating if not avoiding damage to nearby prostate structures or the urethra. The treatment may be used by itself, or in combination with other known treatments. The treatment may be used to administer a composition to a subject in need thereof.

In view of the foregoing, disclosed herein is a method for delivery of a sustained release formulation (SRF) in an efficacious manner and with less side effects to a patient that known methods of treating BPH. The method uses an apparatus including a needle syringe containing a composition, which includes the SRF (hereinafter “composition” will refer to a composition that includes the SRF, unless specifically noted otherwise). The method includes dispensing one or more unit volumes of composition from the needle syringe at a respective one or more target tissue locations of the prostate. The apparatus is characterized by its K-injectate Rating (KIR) defined by the following relationship:

$$KIR = \frac{μ {L}^{2}}{vD} ( 1 {0}^{- 6} )$$

Where μ is the absolute viscosity (cP) of the composition, L is the needle length (cm) measured from the needle tip to the end of the needle hub, D is the needle lumen diameter (cm), and v is a unit volume (cm³ or cc) of the composition contained in the syringe barrel. Each unit volume represents a maximum volume of the composition dispensed at a single location of the target tissue. In some embodiments the syringe holds a plurality of unit volumes, which enables the dispensing of a unit volume at each of a corresponding plurality of discrete locations of the target tissue to achieve a total volume of injected composition at the target tissue. In some embodiments, the total volume in the syringe barrel is the plurality of unit volumes plus some excess to account for priming the needle dead volume.

There is also disclosed an apparatus for prostate treatment. The apparatus includes a needle syringe and the composition contained within the syringe. The apparatus is adapted to deliver the composition in one or more unit volume increments to a target tissue, as defined by its KIR value.

KIR values for the method, apparatus, medical device, and system according to the disclosure may range from between 10 and 1000, 10 and 300, or 40 and 400. The units of KIR are centipoise per unit area. In some embodiments, the KIR values in this paragraph are useful for transurethral administration using a needle having a needle length of 35 cm to 70 cm.

KIR values for the method, apparatus, medical device, and system according to the disclosure may also range from between 10 and 1200, 100 to 900, or 300 to 600. The units of KIR are centipoise per unit area. In some embodiments, the KIR values in this paragraph are useful for transrectal/transperineal administration using a needle having a needle length of 15 cm to 20 cm.

Prostate treatment according to the disclosure may be described as a prostate treatment having three characteristics: minimal uncontrolled drug diffusion (e.g., as shown in Table 8), sustained release and efficacy, and a unit volume as defined herein. The KIR identifies the needle syringe capable of delivering these one or more unit volumes of a composition, in a safe, repeatable pattern by a health professional and without imposing significant compromises on the composition's ability to achieve minimal uncontrolled drug diffusion, sustained release, and efficacy.

Accordingly, in one aspect, a treatment of prostatic hyperplasia tissue, as provided herein, includes the delivery of a drug or multiple drugs to the tissue, of a subject in need thereof, in a sustained release manner using a needle syringe containing the SRF. The treatment may be used with, or in addition to, treatments involving removal/ablation of tissue, and/or delivery of energy to the tissue and additionally the administering of various agents. Methods according to the disclosure may additionally, or alternatively, be administered after a treatment of BPH according to other methods.

Access to prostatic tissue may be achieved in a transurethral, transrectal, or transperineal manner via an existing body orifice. It may be beneficial and less invasive to access the tissue by either transrectal or transperineal approaches. The advantages with a transrectal or transperineal approach include one or more (1) oral and/or local anesthesia application instead of general anesthesia, (2) less trauma to the urethra tract and less resulting side effects also reducing the need for catheterization, (3) faster recovery time for the patient, (4) familiar treatment for the urologist physician similar to prostate cancer biopsy. For access by transrectal or transperineal approach, guidance may be provided by ultrasound, x-ray, computed tomography, magnetic resonance imaging or other imaging modality. Ultrasound imaging may be beneficial given that ultrasound is utilized for prostate biopsy. The transrectal approach closely mirrors the present prostate ultrasound and biopsy techniques familiar to urologists. Transrectal and transperineal approaches both avoid interaction with the urethra, which limits the caustic effects of urethral procedures therefore minimizing side effects and dysuria associated with currently available BPH procedures.

Alternatively, access to prostatic tissue may be beneficial to access the tissue by a transurethral approach. The advantage with a transurethral approach include one or more (1) ability to visualize optically the urethra tract and obstruction during treatment by cystoscopy, (2) ability to create drug prostate lesions of contiguous and consistent treatment depth across the transition zone urethra, (3) trauma to the urethral tract may be minimized using a flexible scope and/or delivery device, (4) avoids the pain and additional device requirements of transperineal approach, (5) avoids the slight infection risk of transrectal approach, (6) familiar treatment for the urologist physician similar to other BPH procedures such as transurethral resection of prostate (TURP), implant and ablation procedures such as Urolift™ and Rezum™. For access by transurethral approach, guidance may be provided by direct optical imaging, ultrasound, computed tomography, magnetic resonance imaging or other imaging modality. Optical imaging may be beneficial given the ability to directly visualize the urethral obstruction and delivery of the SRF. The transurethral approach closely mirrors the present prostate BPH treatment procedures familiar to urologists. Transurethral approach avoids interaction with the rectum, which limits the slight infection risk of transrectal approach.

The drug portion of the composition may be an anti-inflammatory, anti-proliferative, cytoreductive, cytostatic, and/or cytotoxic drug that would affect the prostate size and gland proliferation. The apparatus enables delivery of one or more drugs into the target tissue. Once delivered to the target tissue, the drug may then release from the SRF in a slow, sustained release fashion, optionally delivered as an initial burst of the drug, followed by a slow, sustained release of the drug to the target tissue. The amount or lack of burst and/or the “slow, sustained release” release period may depend on the drug delivered to the prostate, the SRF properties, and the KIR value, as will be appreciated in view of this disclosure. In some embodiments a slow, sustained release may occur over, e.g., a 24-hour period, 3-7 days, 1-4 weeks, 1 to 12 months, 3 months, or 6 months.

In another aspect there is a system for treating BPH including a needle syringe, the sustained release formulation (SRF), and imaging device for locating a target tissue of the prostate. The optical imaging device, e.g., cystoscope, may be used both as a needle guide to the target tissue and for sizing the prostate transition zone urethral length (TZUL). Once the prostate transition zone urethral length is determined, the number, n, of unit volumes, v, for injection may be determined. A needle syringe according to the disclosure may hold one or a plurality of unit volumes dispensable from the needle to treat the prostate in a controlled manner, as determined by the KIR value for the syringe containing the composition. The KIR value is, in some embodiments, between 10 and 1200, 100 and 900, or 300 and 600.

In another aspect there is a method for making a medical device for treating the prostate, including the steps of combining at least one drug with at least one polymer carrier to form a composition comprising an SRF, wherein the composition is contained within a needle syringe, and wherein the needle syringe containing the composition has a KIR value of between 10 and 1200, 100 and 900, or 300 and 600. In some embodiments, a water-soluble solvent, capable of dissolving the polymer carrier and the at least one drug, is also present with the polymer carrier.

Treatment of BPH by a needle injection, in a small quantity (unit volume) at a plurality of locations in the target tissue, offers several benefits over other methods. Among the benefits are less invasive procedures leading to greater patient acceptance and less complications during patient treatment, less frequent procedures needed, and reduced incidence of drug affecting nearby tissue leading to such outcomes as adverse consequences for urinary or sexual function.

For purposes of this disclosure, the following terms and definitions apply:

The following are examples of the polymer naming nomenclature appearing in the listing of additional disclosed embodiments following the detailed description. Other examples not explicitly spelled out here use the same rationale: PLGA8515A (0.3 dl/g) means poly(lactide-co-glycolide) with a monomer ratio of 85/15, end capped with an acid group (A), and an inherent viscosity of 0.3 dl/g; and PLGA6535E (0.5 dl/g) means poly(lactide-co-glycolide) with a monomer ratio of 65/35, end capped with an ester groups (E), and an inherent viscosity of 0.5 dl/g; and PLGA5050A (0.2 dl/g or 0.1 dl/g) means poly(lactide-co-glycolide) with a monomer ratio of 50/50, end capped with an acid group (E), and an inherent viscosity of 0.2 dl/g; and poly(lactide-co-glycolide) is typically poly(D,L-lactide-co-glycolide) but could also be e.g. any or a mixture of poly(D,L-lactide-co-glycolide), poly(D-lactide-co-glycolide), and poly(L-lactide-co-glycolide).

The terms “about” or “approximately” are defined herein as 30%, 20%, 15%, 10%, 5%, 4%, 3%, 2%, 1.5%, 1%, between 1-2%, 1-3%, 1-5%, or 0.5%-5% less or more than, less than, or more than a stated value, a range or each endpoint of a stated range, or a one-sigma, two-sigma, three-sigma variation from a stated mean or expected value (Gaussian distribution). For example, dl is about d2 means dl is 30%, 20%, 15%, 10%, 5%, 4%, 3%, 2%, 1.5%, 1%, 0% or between 1-2%, 1-3%, 1-5%, or 0.5%-5% different from d2. If dl is a mean value, then d2 is about dl means d2 is within a one-sigma, two-sigma, or three-sigma variance from dl. It is understood that any numerical value, range, or either range endpoint (including, e.g., “approximately none”, “about none”, “about all”, etc.) preceded by the word “about,” “substantially” or “approximately” in this disclosure also describes or discloses the same numerical value, range, or either range endpoint not preceded by the word “about,” “substantially” or “approximately.”

The term “drug” or “agent” as used herein is defined as a therapeutic substance, other than food, used in the prevention, diagnosis, alleviation, treatment, or cure of disease. Unless stated otherwise, “drug” and “agent” shall have the same meaning.

The term “cytostatic” as used herein refers to a drug that is non-toxic to cells but does mitigate cell proliferation and permit cell migration. Cytostatic drugs may include without limitation rapamycin, sirolimus, everolimus, zotarolimus, myolimus, temsirolimus, tacrolimus, macrolide antibiotics, ridaforolimus, biolimus, novolimus, deforolimus, structural derivatives and functional analogues of rapamycin and any macrolide immunosuppressive drug. mTOR/PI3K dual inhibitors may also be utilized including dactolisib, BGT226, SF1126, PKI-587, and NVPBE235. mTORC1/mTORC2 dual inhibitors may also be utilized including sapanisertib, AZD8055, and AZD2014 as derived from morpholino pyrazolopyrimidine.

The term “cytotoxic” as used herein refers to a drug that inhibits cell growth and proliferation such as chemotherapeutics. These drugs may include but are not limited to paclitaxel, taxanes, protaxel, vincristine, etoposide, nocodazole, indirubin, anthracycline derivatives, daunorubicin, daunomycin, tauromustine, bofumustane, carboplatin, carmustine, cisplatin, docetaxel, cabazitaxel, gemcitabine, mitomycin, procarbazine, and plicamycin. These drugs may also be apoptotic such as TGF, topoisomerase inhibitors, including, 10-hydroxycamptothecin, irinotecan, and doxorubicin.

The term “composition” as used herein means a product of mixing or combining various elements or ingredients. Whenever the word “composition” is used, it will be understood that the composition comprises an SRF. The term “polymer composition” however is not a composition that comprises the SRF.

The term “absolute viscosity” as used herein means the viscosity measured relative to the viscosity of a known substance. Unless stated otherwise “absolute viscosity” (as compared to inherent viscosity) is represented by the symbol p. Absolute viscosities may be measured using a calibrated digital rotational viscometer spindle (no. 1 size) with 10 mL solutions in 40 ml polypropylene vials with an approximately 50% submerged spindle solution contact, with measurements made at room temperature conditions.

The term “partial solution” as used herein means a composition comprising a mixture of dissolved drug in SRF and suspension of non-dissolved drug in SRF. The term “solution” as used herein means a composition comprising dissolved drug in SRF and the absence of non-dissolved drug. A solution means the concentration of the drug is below the saturation limit in the solvent.

The term “Depth setting” as used herein means the depth of needle head penetration into the prostate. From a canine preclinical study, it was determined that SRF resulting lesion size was approximately 0.5 cm 30 days after the procedure. Therefore, in an embodiment, a depth setting of the needle of at least 2.5 mm is necessary to maximize the necessary SRF drug prostate effect to treat the prostate BPH. Moreover, it is desired to have a depth setting of about half the lesion size or greater.

The term “unit volume” as used herein refers to the maximum volume (v) of composition for an individual injection when treating BPH. In most cases there is a plurality of unit volumes of a composition per patient. For example, there can be n=2, 3, 4, 5, 6, or more, depending on the size of the prostate transition zone urethra length, which can be determined from ultrasound imaging and/or by direct optical cystoscopy prior to or during treatment. Herein, n is the number of unit volumes. The larger the size of the prostate transition zone urethra length, generally speaking, the higher the number of unit volumes dispensed from a needle at different locations of the prostate. It was found in pre-clinical studies that a prostate treatment using a unit volume of 0.05 ml to 0.1 ml of composition can result in significant reduction in prostate size over a 90 day period. Moreover, it is believed that a unit volume of 0.05 ml to 0.5 ml, 0.05 ml to 0.1 ml, 0.1 to 0.2, or 0.05 to 0.2 ml will also produce positive clinical outcomes with no adverse indications.

From a pre-clinical study in canines with prostate volumes ranging from 19.5 to 44.1 cm³ (average 30 cm³), discussed in greater detail, infra., a unit volume of from 0.050 to 0.100 mL and total injected amount for a prostate for 2 unit volume injections of 0.100 mL and 0.200 mL, respectively, produced the desired result (human prostate volumes are typically between 20 to 80 cubic centimeters) or grams (prostate mass density can be approximated as 1 g/cc). In this study local retention of the drug and drug effect was observed and there was no damage to surrounding organs, and there was reduction in prostate volume at 30 and 90 days post SRF treatment.

Normal adult human prostates weigh approximately 20-25 grams (g). A majority of BPH prostate sizes are 30 g or larger with most ranging between 30 g-80 g while some can be significantly larger. To achieve approximately a >25% reduction in size, the number (n) of unit volumes (v) injected per prostate may be selected based on prostate size. Therefore, to reduce a larger size prostate by the same amount, an increased number of unit volumes may be utilized. For example, the number (n) of unit volumes injected to achieve greater than 15%, or greater than 25% reduction in prostate size may be determined from TABLE 1A.

TABLE 1A

| Prostate Size | # Unit Volumes Injected |
|---|---|
| (g or cm³ [cc]) | Total (in both lobes) |
| 20-40 | 2-4 |
| 40-60 | 2-6 |
| 60-80 | 4-8 |
| >80 | 6-10 |

The term “total volume” of composition (i.e., the sum of n unit volumes of composition injected into the prostate during a single treatment session, defined as within a 1-2 hours of, or within 24 hours of each other) is the total volume of composition administered to the patient and in an effective amount producing a programmed, sustained release and efficacious outcome when treating BPH. This programmed, sustained release and efficacious outcome may be measured using the International Prostate Symptom Score (IPSS). This programmed, sustained release and efficacious outcome may also be measured, more generally, using the relieving Lower urinary tract symptoms (LUTS), which include voiding or obstructive symptoms such as hesitancy, poor and/or intermittent stream, straining, feeling of incomplete bladder emptying, and storage or irritative symptoms such as frequency, urgency, urge incontinence, and nocturia. A total volume is expected to produce more than a 15% reduction in prostate volume, or more than a 25% reduction in prostate volume. According to the pre-clinical study using canine models, prostate volumes were reduced by up to 30% over a 30 day to 90 day period (canine prostate sizes ranged from 19.5 to 44.1 cc, with average being 30 cc at treatment, and averages being 10 cc at 30 and 90 days after treatment). In some embodiments, the number, n, of a unit volume, v, needed to attain a total volume of composition for prostate treatment is related to the prostate volume, PV as n=PV/(200*v) (rounded down to nearest integer). For example, a prostate volume of 30 cc, and unit volume of 0.07 ml gives a value of 2.14, which is then rounded down to n=2.

A majority of human BPH transition zone urethra lengths (TZUL) range from 1.5 cm to 3.5 cm or larger. To achieve approximately a greater than 25% reduction in size, the number (n) of unit volumes (v) injected per prostate may be selected based on prostate TZUL. Therefore, to reduce a larger TZUL by the same amount, an increased number of unit volumes may be utilized. For example, the number (n) of unit volumes injected to achieve greater than 15%, or greater than 25% reduction in prostate size may be determined from TABLE 1B.

TABLE 1B

| Prostate | # Unit Volumes Injected | # Unit Volumes Injected |
|---|---|---|
| TZUL | Total (in each lateral | Total (in median lobe of |
| (cm) | lobe of prostate) | prostate if obstructive) |
| 1.5-2.0 | 1-2 | 1 |
| >2.0-3.0 | 2-3 | 2 |
| >3.0 | 3-4 | 3 |

| | | n (total inc. | | | n (total add | Median Lobe | Total |
|---|---|---|---|---|---|---|---|
| | Unit | 1 per each | Transition | Total | per median | Urethra | Injection |
| n | Volume | lateral lobe | Zone Urethra | Injection | lobe of | Length | Volume |
| (eqn) | (cc) | of prostate) | Length (cm) | Volume (cc) | prostate) | (cm) | (cc) |
| 2 | 0.05 | 2 | 1.5 | 0.1 | 1 | 1.5 | 0.05 |
| 3 | 0.05 | 2 | 1.75 | 0.1 | 1 | 1.75 | 0.05 |
| 3 | 0.05 | 2 | 2 | 0.1 | 1 | 2 | 0.05 |
| 4 | 0.05 | 4 | 2.25 | 0.2 | 2 | 2.25 | 0.1 |
| 4 | 0.05 | 4 | 2.5 | 0.2 | 2 | 2.5 | 0.1 |
| 5 | 0.05 | 4 | 2.75 | 0.2 | 2 | 2.75 | 0.1 |
| 5 | 0.05 | 6 | 3 | 0.3 | 3 | 3 | 0.15 |
| 5 | 0.05 | 6 | 3.25 | 0.3 | 3 | 3.25 | 0.15 |
| 6 | 0.05 | 6 | 3.5 | 0.3 | 3 | 3.5 | 0.15 |
| | | n = TZUL / | | | | | |
| | | (11*v) | | | | | |

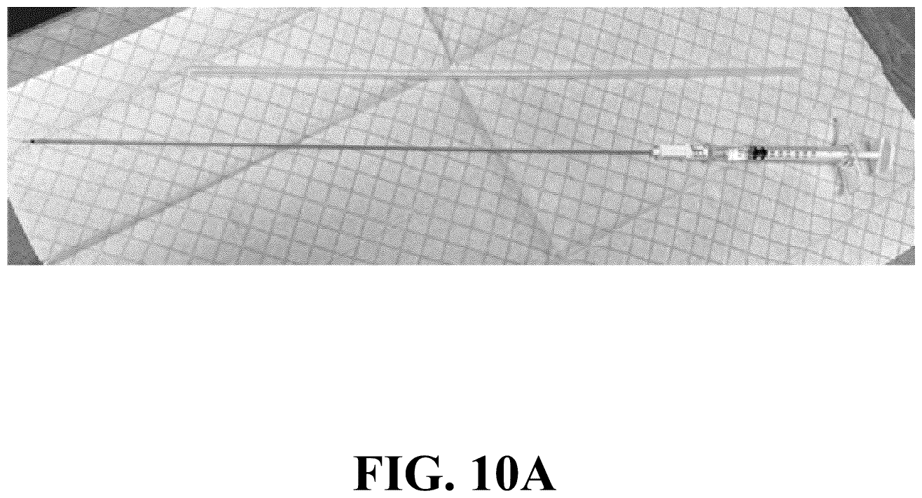

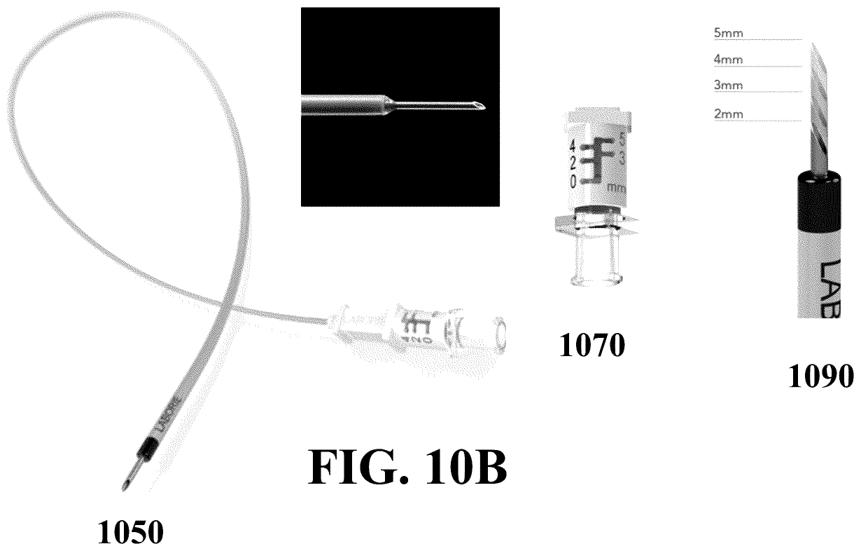

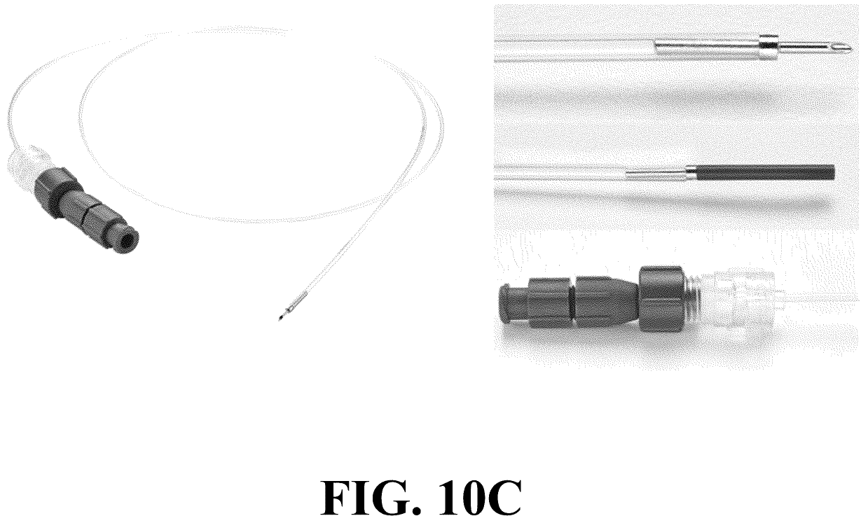

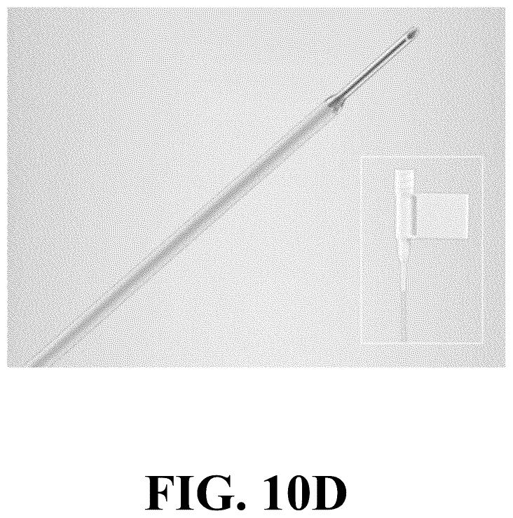

FIG. 10A shows a syringe coupled to a needle having a depth limiter at the end of the needle syringe. FIG. 10B shows a flexible needle—labeled 1050—that can be coupled to a syringe. The tip of the needle, illustrated on the far right side of FIG. 10B—labeled 1090—shows a needle tip having depth limiting indications labeled at 2 mm, 3 mm, 4 mm, and 5 mm. Also shown is adapter—labeled 1070—for controlling the depth of penetration of the needle. FIG. 10C shows another example of a flexible needle for administration a SRF composition set forth herein. FIG. 10D shows a needle tip assembly.

FIGS. 10A, 10B, 10C, and 10D, show examples of commercially available transurethral cystoscopy needles from Laborie (https://www.laborie.com/product/injetak-cystoscopy-needies/), Coloplast (https://iu.coloplast.us/products/bonee-needle) Cook (https://www.cookmedical.com/products/uro_wcin_webds/) that have either fixed or adjustable needle penetration depths. The entire contents of these network addresses, as of Jan. 27, 2023, is herein incorporated by reference in their entirety for all purposes.

In some embodiments, including any of the foregoing, the SRF syringe is connected to the proximal luer hub of the needle and then these needles are inserted into commercially available rigid or flexible cystoscopes (https://medical.olympusamerica.com/procedure/flexible-cystoscopy) to inject or implant the SRF in the prostate via transurethral access under optical guidance from the cystoscope. The cystoscope also allows direct imaging of the urethral obstruction and length of obstruction to enable precise determination of number of unit volumes to inject and location of each injection. The entire contents of the network address, in this paragraph, as of Jan. 27, 2023, is herein incorporated by reference in its entirety for all purposes.

The unit volume to TZUL correlation is also important to create contiguous SRF drug lesions across the TZUL in order to treat the LUTS resulting from BPH for efficacy and symptom relief to the patient and also not create additional trauma and/or pain. It is known that excessive necrosis and lesion sizes in the prostate can lead to prostate swelling and urethral compression and worsening of BPH symptoms during the lesion healing phase of approximately 30 days or more prior to symptom relief, which can delay symptom relief by 90 days or more. The instant disclosure sets forth methods and apparatus to delivery multiple precise unit volumes of a SRF per the TZUL correlation and at precise depth settings to obtain patient symptom relief by 30 days in absence of prostate swelling and urethral compression due to excess drug lesion effects.

The term “sustained release formulation (SRF)” as used herein refers to a substance for treating BPH, the substance including a drug (or drugs), solvent for the drug and carrier for the drug(s) or drug carrier comprising a polymer composition administered to the target tissue in liquid, gel or solid form using a delivery vehicle, such as a needle syringe, whereupon local delivery of the composition (comprising the SRF) to the target tissue the SRF is effective in producing a sustained release of the drug(s) to the targeted tissue of the prostate, thereby producing an efficacious result over a period of time, e.g., from 14 days, 30 days, 1 to 3 months, up to 6 months, up to 12 months, or up to 2 years following treatment and with minimal uncontrolled drug diffusion. Other substances may also be present in the composition (e.g., an ultrasound/echoing enhancing medium or other imaging enhancing depending on the imaging modality used). A programmed, sustained release of, e.g., from 14 days or 1 to 12 months for substantially all of the drug to dissipate from the carrier is achieved by selection of drug carrier (polymer composition) and/or modifying the morphology and mechanical properties, the polymer/drug ratio, and controlling the physical shape/dimensions (volume) of the SRF and/or composition that is delivered to the target tissue. Other factors influencing a release rate are described in greater detail, below.

The term “target tissue” as used herein is defined as prostate tissue to include the transition zone, peripheral zone and central zone of the prostate, and the prostate. Also, to include treatment of prostate lateral lobes and medial lobe by a transurethral approach.

The most common types of prostate treatment are treatments for cancerous or pre-cancerous conditions (i.e., non-malignant tumors) and enlarged prostate, more commonly known as benign prostatic hyperplasia/hypertrophy (BPH). The tissue types are very different between treatments for cancer or pre-cancerous tumors vs. BPH, which calls for different formulations of a similar drug that may be effective in treating either condition. Most notably, the volume of drug (on a per-volume of prostate and/or TZUL basis) at a specific time point is very different when treating a tumor or cancer than when treating BPH. The TZUL correlation is not valid given that treatment of cancerous tissue requires excessive cellular necrosis to eliminate the cancerous cells and prevent spread and resulting risk of swelling due to inflammation is of less concern when treating cancer. Finally, when treating for cancer or tumors, one may want to have the full drug released from a carrier quickly, i.e., within the first 24 hours of injecting the composition into the patient. A slow release, in contrast, means that drug is released at a minimum of a time period of 1-3 month from when the composition was injected into the patient. This creates an extended drug effect that does not cause additional damage and swelling to the prostate. Unlike a cancer treatment, the goal of which may to be remove all cancerous tissue, even at the expense of some healthy tissue, the instant disclosure describes treatments in which a sufficient volume reduction of affected tissue is achieved without also causing excessive damage to surrounding tissue.

In some embodiments a patient to be treated has cancer in another part of the body (other than the prostate) but is being treated for BPH according to the disclosure. In some embodiments the patient does not have cancer in another part of the body and is being treated for BPH.

In some embodiments, including any of the foregoing, the methods herein include administering a SRF or composition described herein to a subject in need thereof and wherein the subject does not have prostate cancer.

In some other embodiments, including any of the foregoing, the methods herein include administering a SRF or composition described herein to a subject in need thereof who does not have cancer.

In yet other embodiments, including any of the foregoing, the methods herein include administering a SRF or composition described herein to a subject in need thereof in which the SRF is not efficacious against a cancer that the subject has.

Procedures for treating BPH have demonstrated varying levels of efficacy and are often accompanied by undesirable or adverse effects. For example, TURP produces improved efficacy and improvement in urinary flow rate and symptom score (IPSS) but is invasive with significant side effects including incontinence, urgency, dysuria, acute retention, stricture, ejaculation dysfunction and sexual dysfunction. Water vapor therapy and PUL have demonstrated less sexual dysfunction side effects but are limited to use in smaller BPH prostates less than 80 ml and have shown less efficacy with non-responders and higher retreatment rates compared to TURP. Furthermore, these procedures are invasive and require transurethral access with large rigid delivery devices of 20 Fr (French size catheter) or more and catheter placement after treatment due to excessive prostate injury and swelling during healing.

Less invasive targeted drug delivery approaches to the prostate zone have been attempted by the transurethral, transrectal or transperineal routes such as pore forming proteins and peptides in saline formulations with single dosages but demonstrated limited efficacy versus saline placebo in randomized clinical trials. See Indian J Urol. 2008 July-September; 24(3): 329-335. doi: 10.4103/0970-1591.42613, PMCID: PMC2684358, PMID: 19468462; Injection therapy for prostatic disease: A renaissance concept. Arash M. Saemi, Jeffrey B. Folsom, and Mark K. Plante. Additionally, alcohol, botox (onabotulinumtoxinA) or medications injected into the prostate have been ineffective. Alcohol single injection is very caustic and poorly controls the area of delivery. Medication injection into the prostate has also been ineffective as it is given in a single dose with poor effect. Other attempts to treat prostate using similar drugs and/or peptide drugs have been used. If injected, the injectate did not include a sustained release formulation of the drug and thus a long acting, efficacious response in the target tissue injected would not be exhibited.

While the foregoing methods may show efficacy in reducing BPH, they either require a more invasive procedure (vs. localized treatment using the instantly disclosed delivery devices and methods herein), more frequent treatment due to diffusion or more generalized treatment of BPH raising the possibility of adverse effects because a comparatively high dosage of the drug is needed to treat the area while accounting for leakage or diffusion of the drug to other areas (i.e., at least 2 to 3 times higher dose of the drug compared to the drug dosage in a unit volume of an SRF). This may often result in a greater total volume of the SRF to the affected area than would be necessary if the instantly disclosed devices and methods were used. Adverse effects may include diminished urinary or sexual function. It is desired to have an effective treatment targeting only the target tissue and nowhere else (e.g., avoiding the bladder, urethra and nerves surrounding the prostate) and to perform the procedure in a less invasive manner for patient acceptance.

With these objectives in mind, the inventors sought to develop a formulation and method that could be delivered by way of local prostate needle injection and that could overcome the foregoing drawbacks with existing procedures for treating BPH. The formulation developed is the SRF, which is described in greater detail below, followed by several embodiments of the SRF. Embodiments of a device and method for delivering the SRF using the device, which involves use of a needle syringe, is also described.

A SRF for treating BPH, according to the disclosure, is preferably delivered transurethrally (i.e., by way of the patient's urethra), transrectally, or transperineally to the target tissue using a needle syringe having a relatively long and narrow needle (for example, a 15 cm to 20 cm length needle, and 0.02 cm to 0.12 cm needle lumen, or 35 cm 45 cm length needle and 0.02 cm to 0.04 cm needle lumen. In some embodiments, a 23 G needle is used. In some embodiments, a needle having a slenderness ratio, defined as needle length divided by lumen diameter. In some embodiments, including any of the foregoing, the slenderness ratio is between 200 and 400 or, more preferably, between 250 and 340 for transrectal, or greater than 850 for transurethral approach. In some embodiments, including any of the foregoing, the slenderness ratio is less than 2100 for transrectal, or for transurethral approach. In some embodiments, including any of the foregoing, the slenderness ratio is 200 to 400. In some other embodiments, including any of the foregoing, the slenderness ratio is 200 to 300. In some embodiments, including any of the foregoing, the slenderness ratio is 300 to 400. In some embodiments, including any of the foregoing, the slenderness ratio is 250 to 350. In some embodiments, including any of the foregoing, the slenderness ratio is 250 to 340. In some embodiments, including any of the foregoing, the slenderness ratio is 250 to 330. In some embodiments, including any of the foregoing, the slenderness ratio is 250 to 320. In some embodiments, including any of the foregoing, the slenderness ratio is 250 to 310. In some embodiments, including any of the foregoing, the slenderness ratio is 250 to 300. In some embodiments, including any of the foregoing, the slenderness ratio is 250 to 290. In some embodiments, including any of the foregoing, the slenderness ratio is 250 to 280. In some embodiments, including any of the foregoing, the slenderness ratio is 250 to 270. In some embodiments, including any of the foregoing, the slenderness ratio is 250 to 260. In some embodiments, including any of the foregoing, the slenderness ratio is 260 to 350. In some embodiments, including any of the foregoing, the slenderness ratio is 270 to 350. In some embodiments, including any of the foregoing, the slenderness ratio is 280 to 350. In some embodiments, including any of the foregoing, the slenderness ratio is 290 to 350. In some embodiments, including any of the foregoing, the slenderness ratio is 300 to 350. In some embodiments, including any of the foregoing, the slenderness ratio is 310 to 350. In some embodiments, including any of the foregoing, the slenderness ratio is 320 to 350. In some embodiments, including any of the foregoing, the slenderness ratio is 330 to 350. In some embodiments, including any of the foregoing, the slenderness ratio is 340 to 350. In some embodiments, including any of the foregoing, the slenderness ratio is 260 to 340. In some embodiments, including any of the foregoing, the slenderness ratio is 270 to 340. In some embodiments, including any of the foregoing, the slenderness ratio is 280 to 340. In some embodiments, including any of the foregoing, the slenderness ratio is 290 to 340. In some embodiments, including any of the foregoing, the slenderness ratio is 300 to 340. In some embodiments, including any of the foregoing, the slenderness ratio is 310 to 340. In some embodiments, including any of the foregoing, the slenderness ratio is 320 to 340. In some embodiments, including any of the foregoing, the slenderness ratio is 330 to 340. In some embodiments, including any of the foregoing, the slenderness ratio is 340 to 340. The fluid properties and injectate volume requirements of the composition using this type of needle were initially examined to determine whether there were any limitations, special considerations, or modifications needed to the SRF (and/or delivery device) for delivery of this injectate to a prostate.

In certain embodiments, different needles will be used for different modes of administration. For example, in one embodiment, the mode of administration is transurethral, and the needle length is 35-70 cm. For example, in one embodiment, the mode of administration is transperineal and the needle length is 10-20 cm. For example, in one embodiment, the mode of administration is transrectal and the needle length is 10-20 cm.

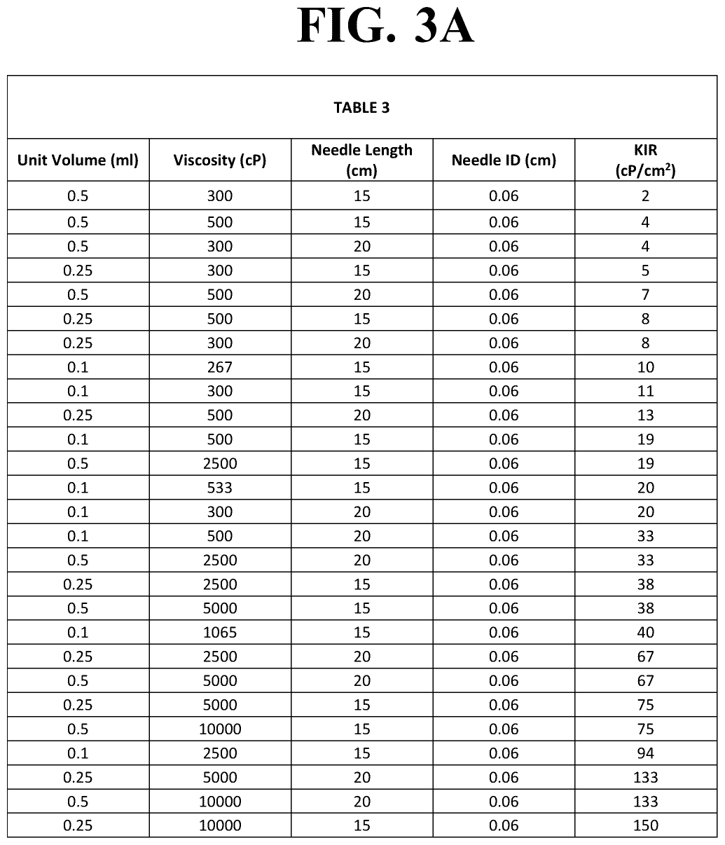

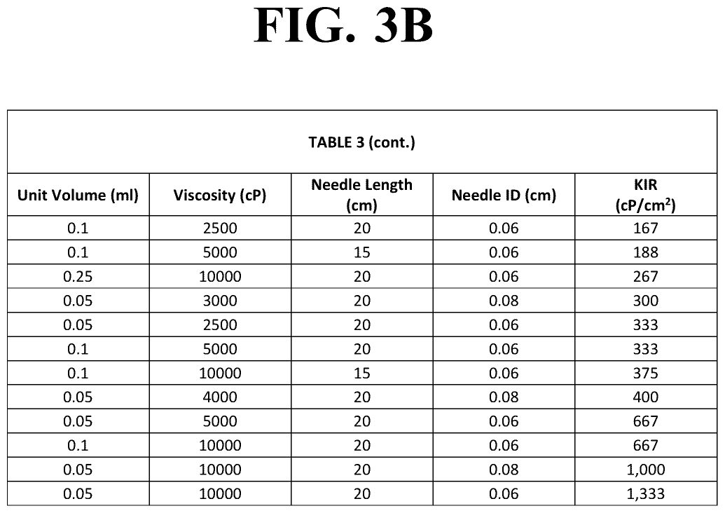

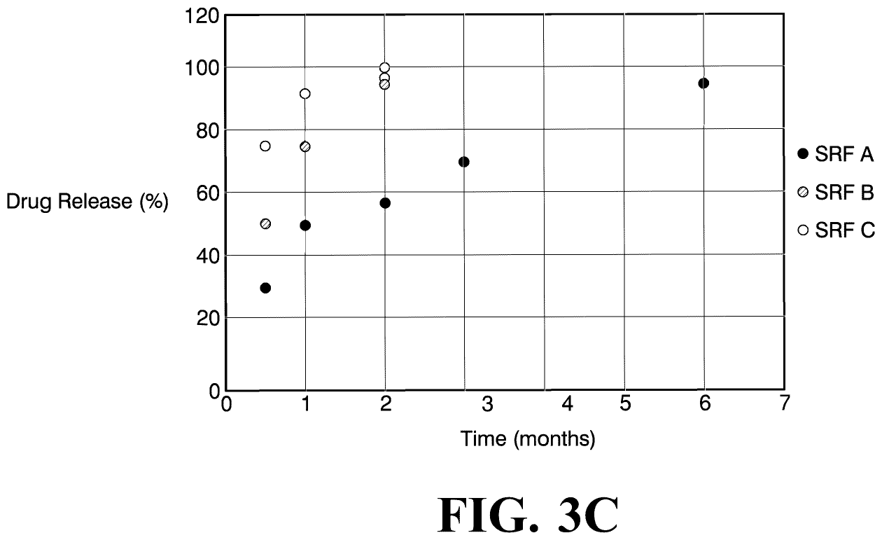

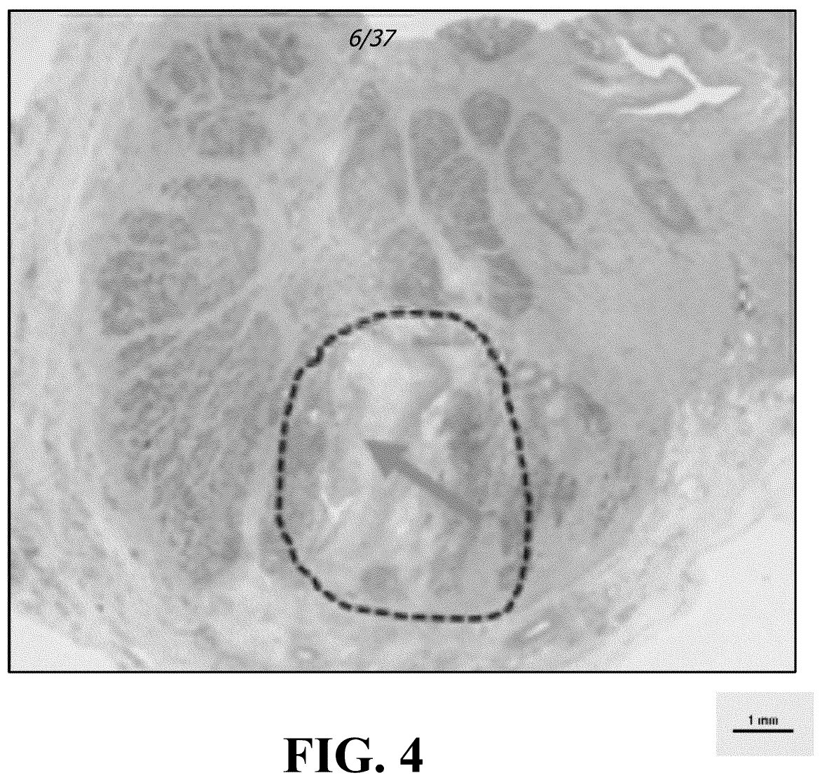

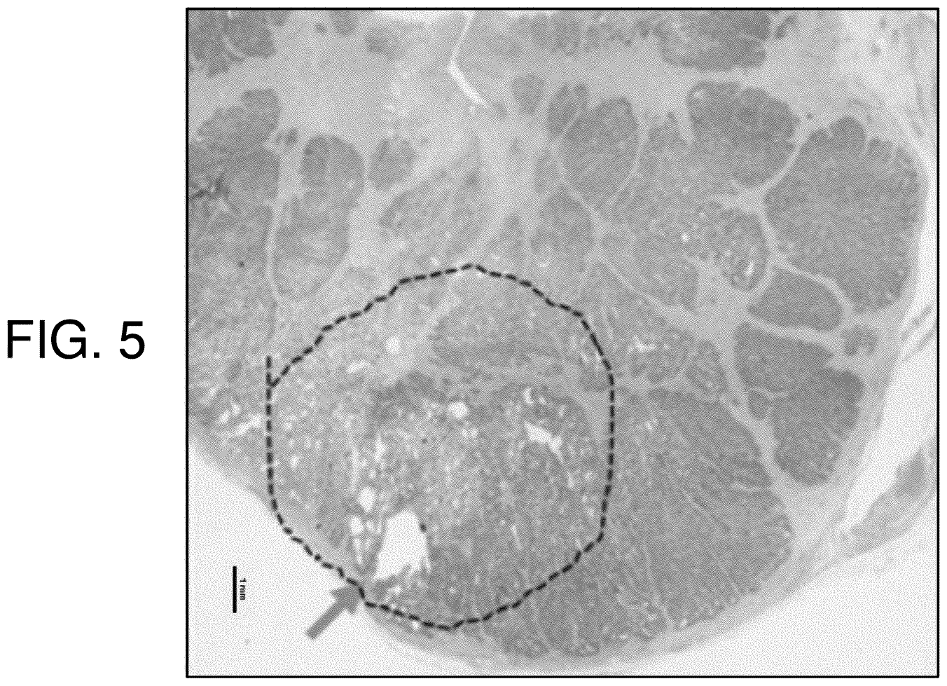

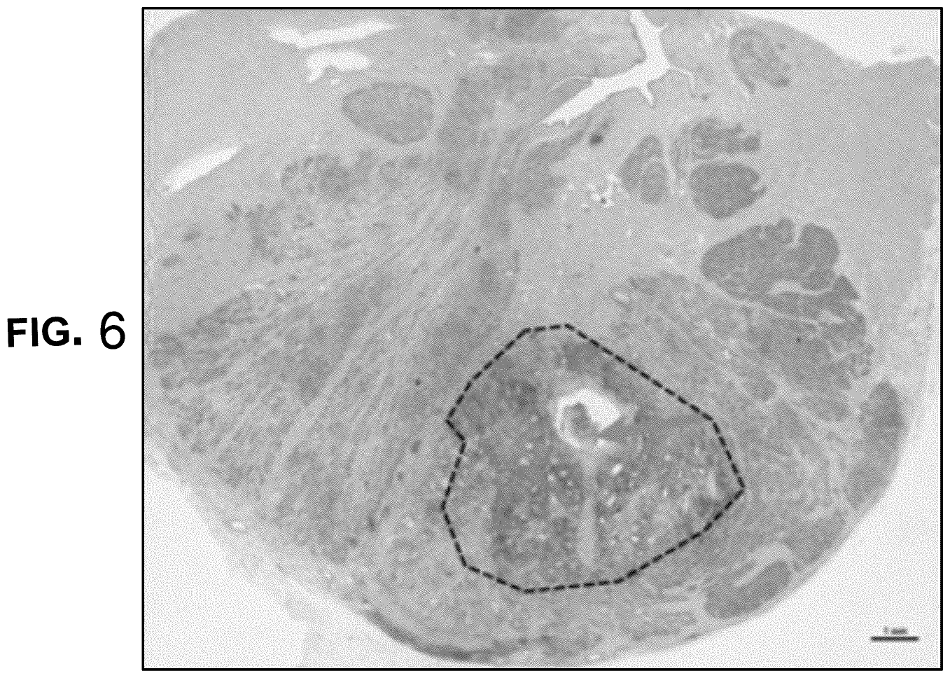

In some embodiments, when the mode of administration is transperineal or transrectal, KIR ranges substantially as shown in FIGS. 3A and 3B.

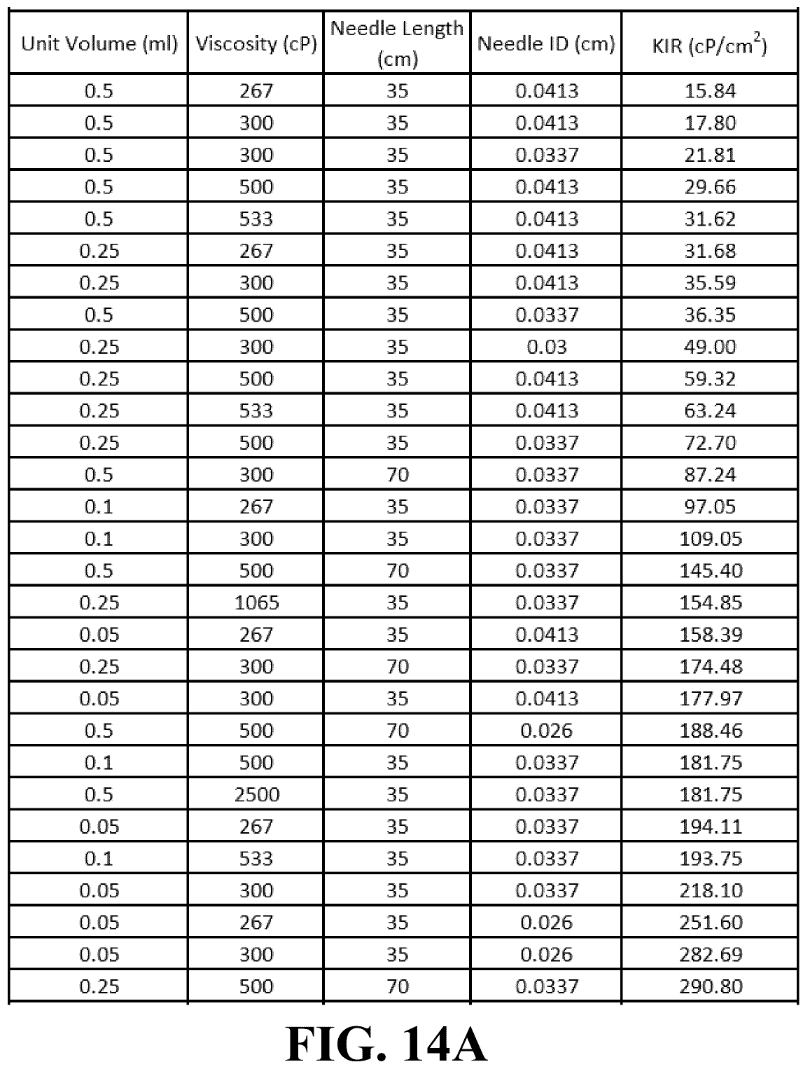

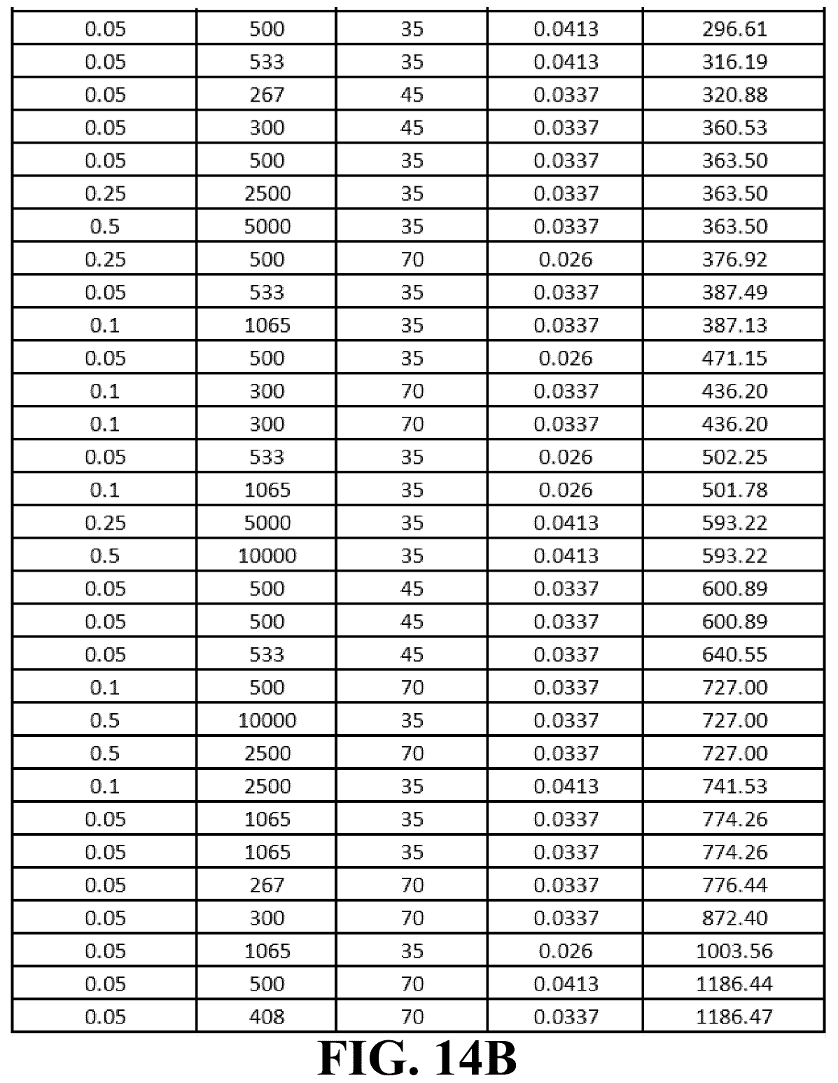

In some embodiments, when the mode of administration is transurethral, KIR ranges substantially as shown in FIGS. 14A and 14B.

In some embodiments, when the mode of administration is transrectal or transperineal, KIR ranges from 2 to 1300. In some embodiments, the needle length is 10 cm to 20 cm. In some embodiments, the needle length is 15 cm to 20 cm.

In some embodiments, when the mode of administration is transurethral, KIR ranges from 16 to 1200. In some embodiments, the needle length is 35 cm to 70 cm.

The question asked, by the inventors of the instant disclosure, was whether the SRF could be delivered to the prostate on a consistent, repeatable basis to ensure a total volume by prostate size is substantially met, but not exceeded, by an administering health professional, to achieve the desired outcome (reduction in prostate volume, over a period of time by a programmed, sustained release of a drug) and without adverse indications (e.g., infection, post-treatment pain or discomfort, drug diffusion into nearby areas such as the urethra or bladder). The inventors found, after extensive bench testing and evaluation of several trials of both delivery device and candidate SRF formulations, a needle syringe suited to deliver the SRF in the manner sought by the inventors.

A prostate treatment according to the disclosure may be described as a prostate treatment having three characteristics:

1. MINIMAL UNCONTROLLED DRUG DIFFUSION: Obtaining sufficient solidification or gelation of the SRF in a relatively short period of time upon contact with the prostate, so that the SRF remains at the target tissue when the needle is removed and, hence, little or no drug diffusion takes place possibly affecting unintended, adjacent tissue such as the urethra or bladder (e.g., as demonstrated by the little or no drug diffusion outside of the prostate for the canine models studied, see TABLE 8).

2. SUSTAINED RELEASE & EFFICACY: Enabling a programmed release of a drug over time from the SRF (see, e.g., FIG. 3C, TABLE 4A and TABLE 4B). The SRF is delivered in a sufficient total volume to enable a sustained release of a drug for prolonged treatment of the target tissue to achieve the efficacious result of reduced prostate size.

3. LOW UNIT VOLUME: Delivery of the composition total volume to the prostate as one or more unit volumes (i.e., “unit volume” as defined herein) using a needle syringe. This manner of delivery avoids causing additional acute pressure on the urethra, which may cause additional discomfort for the patients immediately after or shortly after the procedure, or to avoid excessive swell due to uptake of fluids from the surrounding tissue. Additionally, a low unit volume of the composition avoids pressure at the injection site, which can mitigate against composition flow to surrounding tissues and organs and/or backflow of the composition through the delivery device or through the needle track once the needle is removed, and/or inhibiting the composition from being expelled from the target tissue due to pressure buildup. It can also be desirable to have a unit volume, which is less than the total volume administered, injected at several discrete, nearby locations to achieve the targeted clinical outcome, as this can optimize diffusion of a drug relative to diffusion from a large volume injected in one location.

The factors influencing, and manner by which the inventors arrived at, the SRF formulations and compositions that enable a prostate treatment with these characteristics will now be discussed. More specific examples of embodiments of an SRF are provided later.

Drug to Polymer Ratio. The drug to polymer ratio for the SRF formulation was investigated, as well as the drug to solvent ratio. Too low of a drug to polymer ratio is not attractive. If the drug dosage is too low, then it results in the local drug concentrations being too low. Too high of a drug to polymer ratio results in the drug releasing from the SRF too fast which limits the longer term local drug release and also leads to surrounding tissue and/or systemic drug exposure.

Polymer to Solvent Ratio and Polymer Molecular Weight. The polymer to solvent ratio and polymer molecular weight associated with the SRF formulation were investigated. Too low of a polymer to solvent ratio is not attractive. If the polymer concentration and or molecular weight are too low, then it results in slow gelation and the drug releasing from the SRF too fast which limits the longer-term local drug release and also leads to surrounding tissue and/or systemic drug exposure. As discussed in greater detail below, the desire to achieve a high gelation rate was balanced against the need for deliverability using a needle characterized by a high slenderness ratio. Too high of a polymer to solvent ratio and molecular weight results in a composition having a high viscosity, which requires to be delivered with needles characterized by low slenderness ratios and in relatively large volumes. But the same polymer to solvent ratio and molecular weight can pose a significant challenge to deliver in unit volume amounts using needles characterized by a high slenderness ratio, in addition to the challenge of determining whether that selected polymer to solvent ratio and molecular weight produces the desired gelation rate without also producing too-slow of a drug release. It was these types of trade-offs or balances that led the inventors to identify an optimal combination of needle and composition, which is represented by the KIR.

It should be noted that the above-mentioned ratios and polymer molecular weights (examples provided below) are easily determined for the clinical use intended here; rather, it required lengthy investigation and discovery in order to arrive at the desired localized efficacy and effectiveness specifically tailored to satisfy all three characteristics of BPH treatment.

It is desired to have a programmed, sustained release of, e.g., from 14 days or 1 month to 12 months for substantially all of the drug to dissipate from the carrier. This requires the selection of the drug carrier (polymer composition) and/or modifying the morphology and mechanical properties, the polymer/drug ratio, controlling the physical shape/dimensions (volume) of the SRF and/or composition delivered to the target tissue. Other factors affecting the release rate include:
in-vivo polymer swelling (controlled by polymer structure, e.g., monomers selection, monomer ratios, molecular weight, end groups, and porosity/morphology).
porosity/morphology (controlled by, e.g., polymer structure and concentration, polymer/solvent ratio and miscibility, and polymer/drug ratio). If there is less drug than polymer, for example, then the drug may be trapped longer.
how fast the materials gel and whether the SRF is above or below the wet glass transition temperature—transition from liquid to solid using water soluble or insoluble solvents (fast gelation leads to faster initial release/burst). The speed of gelation depends on polymer structure, molecular weight, and concentration, polymer/solvent ratio and miscibility.
polymer degradation (controlled by polymer structure e.g., monomers selection, monomer ratios, molecular weight, end groups, and porosity/morphology).
drug/polymer miscibility and polarity of solvent, and
molecular weight and lipophilicity of drug.

TABLE 2
Examples of constituents by percent by volume
(% vol) in a composition comprising SRF

| | | | SRF (drug & | SRF |
|---|---|---|---|---|
| Example | Drug | Polymer | polymer %) | (solvent %) |
| 1 | Paclitaxel | PLGA 50/50 | 52.1% | NMP 47.8% |
| | (4.3%) | (47.8%) | (polymer + | |
| | | | drug) | |
| 2 | Sirolimus | PLGA 85/15 | 52.1% | NMP 47.8% |
| | (4.3%) | (47.8%) | (polymer + | |
| | | | drug) | |
| 3 | Paclitaxel | PLGA 50/50 | 51.8% | NMP 48.2% |
| | (3.6%) | (48.2%) | (polymer + | |
| | | | drug) | |
| 4 | Sirolimus | PLGA 85/15 | 51.8% | NMP 48.3% |
| | (3.4%) | (48.3%) | (polymer + | |
| | | | drug) | |
| 5 | Paclitaxel | PLGA 50/50 | 52.5% | NMP 47.5% |
| | (5.0%) | (47.5%) | (polymer + | |
| | | | drug) | |
| 6 | Sirolimus | PLGA 85/15 | 52.5% | NMP 47.5% |
| | (5.0%) | (47.5%) | (polymer + | |
| | | | drug) | |
| 7 | Sirolimus | PLGA 50/50 | 53.5% | NMP 46.5% |
| | (7.0%) | (46.5%) | (polymer + | |
| | | | drug) | |
| 8 | Sirolimus | PLGA 85/15 | 53.5% | NMP 46.5% |
| | (7.0%) | (46.5%) | (polymer + | |
| | | | drug) | |
| 9 | Paclitaxel | PLGA 50/50 | 53.5% | NMP 46.5% |
| | (7.0%) | (46.5%) | (polymer + | |
| | | | drug) | |
| 10 | Paclitaxel | PLGA 85/15 | 53.5% | NMP 46.5% |
| | (7.0%) | (46.5%) | (polymer + | |
| | | | drug) | |
| 11 | Sirolimus | PLGA 50/50 | 45.5% | NMP 54.5% |
| | (8.0%) | (37.5%) | (polymer + | |
| | | | drug) | |
| 12 | Sirolimus | PLGA 85/15 | 45.5% | NMP 54.5% |
| | (8.0%) | (37.5%) | (polymer + | |
| | | | drug) | |
| 13 | Paclitaxel | PLGA 50/50 | 45.5% | NMP 54.5% |
| | (8.0%) | (37.5%) | (polymer + | |
| | | | drug) | |
| 14 | Paclitaxel | PLGA 85/15 | 45.5% | NMP 54.5% |
| | (8.0%) | (37.5%) | (polymer + | |
| | | | drug) | |
| 15 | Sirolimus | PLGA 50/50 | 49.0% | NMP 51.0% |
| | (9.0%) | (40.0%) | (polymer + | |
| | | | drug) | |
| 16 | Sirolimus | PLGA 85/15 | 49.0% | NMP 51.0% |
| | (9.0%) | (40.0%) | (polymer + | |
| | | | drug) | |
| 17 | Paclitaxel | PLGA 50/50 | 49.0% | NMP 51.0% |
| | (9.0.%) | (40.0%) | (polymer + | |
| | | | drug) | |
| 18 | Paclitaxel | PLGA 85/15 | 49.0% | NMP 51.0% |
| | (9.0%) | (40.0%) | (polymer + | |
| | | | drug) | |
| 19 | Sirolimus | PLGA 50/50 | 50.0% | NMP 50.0% |
| | (10.0%) | (40.0%) | (polymer + | |
| | | | drug) | |
| 20 | Sirolimus | PLGA 85/15 | 50.0% | NMP 50.0% |
| | (10.0%) | (40.0%) | (polymer + | |
| | | | drug) | |
| 21 | Paclitaxel | PLGA 50/50 | 50.0% | NMP 50.0% |
| | (10.0%) | (40.0%) | (polymer + | |
| | | | drug) | |
| 22 | Paclitaxel | PLGA 85/15 | 50.0% | NMP 50.0% |
| | (10.0%) | (40.0%) | (polymer + | |
| | | | drug) | |
| 23 | Sirolimus | PLGA 50/50 | 47.0% | NMP 53.0% |
| | (12.0%) | (35.0%) | (polymer + | |
| | | | drug) | |
| 24 | Sirolimus | PLGA 85/15 | 47.0% | NMP 53.0% |
| | (12.0%) | (35.0%) | (polymer + | |
| | | | drug) | |
| 25 | Paclitaxel | PLGA 50/50 | 47.0% | NMP 53.0% |
| | (12.0%) | (35.0%) | (polymer + | |
| | | | drug) | |
| 26 | Paclitaxel | PLGA 85/15 | 47.0% | NMP 53.0% |
| | (12.0%) | (35.0%) | (polymer + | |
| | | | drug) | |
| 27 | Docetaxel | PLGA 50/50 | 45.5% | NMP 54.5% |
| | (8.0%) | (37.5%) | (polymer + | |
| | | | drug) | |
| 28 | Docetaxel | PLGA 85/15 | 45.5% | NMP 54.5% |
| | (8.0%) | (37.5%) | (polymer + | |
| | | | drug) | |
| 29 | Docetaxel | PLGA 50/50 | 49.0% | NMP 51.0% |
| | (9.0%) | (40.0%) | (polymer + | |
| | | | drug) | |
| 30 | Docetaxel | PLGA 85/15 | 49.0% | NMP 51.0% |
| | (9.0%) | (40.0%) | (polymer + | |
| | | | drug) | |
| 31 | Docetaxel | PLGA 50/50 | 49.0% | NMP 51.0% |
| | (9.0.%) | (40.0%) | (polymer + | |
| | | | drug) | |
| 32 | Docetaxel | PLGA 85/15 | 49.0% | NMP 51.0% |
| | (9.0%) | (40.0%) | (polymer + | |
| | | | drug) | |
| 33 | Docetaxel | PLGA 50/50 | 50.0% | NMP 50.0% |
| | (10.0%) | (40.0%) | (polymer + | |
| | | | drug) | |
| 34 | Docetaxel | PLGA 85/15 | 50.0% | NMP 50.0% |
| | (10.0%) | (40.0%) | (polymer + | |
| | | | drug) | |
| 35 | Docetaxel | PLGA 50/50 | 50.0% | NMP 50.0% |
| | (10.0%) | (40.0%) | (polymer + | |
| | | | drug) | |
| 36 | Docetaxel | PLGA 85/15 | 50.0% | NMP 50.0% |
| | (10.0%) | (40.0%) | (polymer + | |
| | | | drug) | |
| 37 | Docetaxel | PLGA 50/50 | 47.0% | NMP 53.0% |
| | (12.0%) | (35.0%) | (polymer + | |
| | | | drug) | |
| 38 | Docetaxel | PLGA 85/15 | 47.0% | NMP 53.0% |
| | (12.0%) | (35.0%) | (polymer + | |
| | | | drug) | |
| 39 | Docetaxel | PLGA 50/50 | 47.0% | NMP 53.0% |
| | (12.0%) | (35.0%) | (polymer + | |
| | | | drug) | |
| 40 | Docetaxel | PLGA 85/15 | 47.0% | NMP 53.0% |
| | (12.0%) | (35.0%) | (polymer + | |
| | | | drug) | |
| 41 | Everolimus | PLGA 50/50 | 45.5% | NMP 54.5% |
| | (8.0%) | (37.5%) | (polymer + | |
| | | | drug) | |
| 42 | Everolimus | PLGA 85/15 | 45.5% | NMP 54.5% |
| | (8.0%) | (37.5%) | (polymer + | |
| | | | drug) | |
| 43 | Everolimus | PLGA 50/50 | 49.0% | NMP 51.0% |
| | (9.0%) | (40.0%) | (polymer + | |
| | | | drug) | |
| 44 | Everolimus | PLGA 85/15 | 49.0% | NMP 51.0% |
| | (9.0%) | (40.0%) | (polymer + | |
| | | | drug) | |
| 45 | Everolimus | PLGA 50/50 | 49.0% | NMP 51.0% |
| | (9.0.%) | (40.0%) | (polymer + | |
| | | | drug) | |
| 46 | Everolimus | PLGA 85/15 | 49.0% | NMP 51.0% |
| | (9.0%) | (40.0%) | (polymer + | |
| | | | drug) | |
| 47 | Everolimus | PLGA 50/50 | 50.0% | NMP 50.0% |
| | (10.0%) | (40.0%) | (polymer + | |
| | | | drug) | |
| 48 | Everolimus | PLGA 85/15 | 50.0% | NMP 50.0% |
| | (10.0%) | (40.0%) | (polymer + | |
| | | | drug) | |
| 49 | Everolimus | PLGA 50/50 | 50.0% | NMP 50.0% |
| | (10.0%) | (40.0%) | (polymer + | |
| | | | drug) | |
| 50 | Everolimus | PLGA 85/15 | 50.0% | NMP 50.0% |
| | (10.0%) | (40.0%) | (polymer + | |
| | | | drug) | |
| 51 | Docetaxel | PLGA 50/50 | 47.0% | NMP 53.0% |
| | (12.0%) | (35.0%) | (polymer + | |
| | | | drug) | |
| 52 | Docetaxel | PLGA 85/15 | 47.0% | NMP 53.0% |
| | (12.0%) | (35.0%) | (polymer + | |
| | | | drug) | |
| 53 | Carmustine | PLGA 50/50 | 45.5% | NMP 54.5% |
| | (8.0%) | (37.5%) | (polymer + | |
| | | | drug) | |
| 54 | Carmustine | PLGA 85/15 | 45.5% | NMP 54.5% |
| | (8.0%) | (37.5%) | (polymer + | |
| | | | drug) | |
| 55 | Carmustine | PLGA 50/50 | 49.0% | NMP 51.0% |
| | (9.0%) | (40.0%) | (polymer + | |
| | | | drug) | |
| 56 | Carmustine | PLGA 85/15 | 49.0% | NMP 51.0% |
| | (9.0%) | (40.0%) | (polymer + | |
| | | | drug) | |
| 57 | Carmustine | PLGA 50/50 | 49.0% | NMP 51.0% |
| | (9.0.%) | (40.0%) | (polymer + | |
| | | | drug) | |
| 58 | Carmustine | PLGA 85/15 | 49.0% | NMP 51.0% |
| | (9.0%) | (40.0%) | (polymer + | |
| | | | drug) | |
| 59 | Carmustine | PLGA 50/50 | 50.0% | NMP 50.0% |
| | (10.0%) | (40.0%) | (polymer + | |
| | | | drug) | |
| 60 | Carmustine | PLGA 85/15 | 50.0% | NMP 50.0% |
| | (10.0%) | (40.0%) | (polymer + | |
| | | | drug) | |
| 61 | Carmustine | PLGA 50/50 | 50.0% | NMP 50.0% |
| | (10.0%) | (40.0%) | (polymer + | |
| | | | drug) | |
| 62 | Carmustine | PLGA 85/15 | 50.0% | NMP 50.0% |
| | (10.0%) | (40.0%) | (polymer + | |
| | | | drug) | |
| 63 | Carmustine | PLGA 50/50 | 47.0% | NMP 53.0% |
| | (12.0%) | (35.0%) | (polymer + | |
| | | | drug) | |
| 64 | Carmustine | PLGA 85/15 | 47.0% | NMP 53.0% |
| | (12.0%) | (35.0%) | (polymer + | |
| | | | drug) | |
| 65 | Mitomycin | PLGA 50/50 | 45.5% | NMP 54.5% |
| | (8.0%) | (37.5%) | (polymer + | |
| | | | drug) | |
| 66 | Mitomycin | PLGA 85/15 | 45.5% | NMP 54.5% |
| | (8.0%) | (37.5%) | (polymer + | |
| | | | drug) | |
| 67 | Mitomycin | PLGA 50/50 | 49.0% | NMP 51.0% |
| | (9.0%) | (40.0%) | (polymer + | |
| | | | drug) | |
| 68 | Mitomycin | PLGA 85/15 | 49.0% | NMP 51.0% |
| | (9.0%) | (40.0%) | (polymer + | |
| | | | drug) | |
| 69 | Mitomycin | PLGA 50/50 | 49.0% | NMP 51.0% |
| | (9.0.%) | (40.0%) | (polymer + | |
| | | | drug) | |
| 70 | Mitomycin | PLGA 85/15 | 49.0% | NMP 51.0% |
| | (9.0%) | (40.0%) | (polymer + | |
| | | | drug) | |
| 71 | Mitomycin | PLGA 50/50 | 50.0% | NMP 50.0% |
| | (10.0%) | (40.0%) | (polymer + | |
| | | | drug) | |
| 72 | Mitomycin | PLGA 85/15 | 50.0% | NMP 50.0% |
| | (10.0%) | (40.0%) | (polymer + | |
| | | | drug) | |
| 73 | Mitomycin | PLGA 50/50 | 50.0% | NMP 50.0% |
| | (10.0%) | (40.0%) | (polymer + | |
| | | | drug) | |
| 74 | Mitomycin | PLGA 85/15 | 50.0% | NMP 50.0% |
| | (10.0%) | (40.0%) | (polymer + | |
| | | | drug) | |
| 75 | Mitomycin | PLGA 50/50 | 47.0% | NMP 53.0% |
| | (12.0%) | (35.0%) | (polymer + | |
| | | | drug) | |
| 76 | Mitomycin | PLGA 85/15 | 47.0% | NMP 53.0% |
| | (12.0%) | (35.0%) | (polymer + | |
| | | | drug) | |
| 77 | Tacrolimus | PLGA 50/50 | 45.5% | NMP 54.5% |
| | (8.0%) | (37.5%) | (polymer + | |
| | | | drug) | |
| 78 | Tacrolimus | PLGA 85/15 | 45.5% | NMP 54.5% |
| | (8.0%) | (37.5%) | (polymer + | |
| | | | drug) | |
| 79 | Tacrolimus | PLGA 50/50 | 49.0% | NMP 51.0% |
| | (9.0%) | (40.0%) | (polymer + | |
| | | | drug) | |
| 80 | Tacrolimus | PLGA 85/15 | 49.0% | NMP 51.0% |
| | (9.0%) | (40.0%) | (polymer + | |
| | | | drug) | |
| 81 | Tacrolimus | PLGA 50/50 | 49.0% | NMP 51.0% |
| | (9.0.%) | (40.0%) | (polymer + | |
| | | | drug) | |
| 82 | Tacrolimus | PLGA 85/15 | 49.0% | NMP 51.0% |
| | (9.0%) | (40.0%) | (polymer + | |
| | | | drug) | |
| 85 | Tacrolimus | PLGA 50/50 | 50.0% | NMP 50.0% |
| | (10.0%) | (40.0%) | (polymer + | |
| | | | drug) | |
| 86 | Tacrolimus | PLGA 85/15 | 50.0% | NMP 50.0% |
| | (10.0%) | (40.0%) | (polymer + | |
| | | | drug) | |
| 87 | Tacrolimus | PLGA 50/50 | 50.0% | NMP 50.0% |
| | (10.0%) | (40.0%) | (polymer + | |
| | | | drug) | |
| 88 | Tacrolimus | PLGA 85/15 | 50.0% | NMP 50.0% |
| | (10.0%) | (40.0%) | (polymer + | |
| | | | drug) | |
| 89 | Tacrolimus | PLGA 50/50 | 47.0% | NMP 53.0% |
| | (12.0%) | (35.0%) | (polymer + | |
| | | | drug) | |
| 90 | Tacrolimus | PLGA 85/15 | 47.0% | NMP 53.0% |
| | (12.0%) | (35.0%) | (polymer + | |
| | | | drug) | |
| 91 | Temsirolimus | PLGA 50/50 | 45.5% | NMP 54.5% |
| | (8.0%) | (37.5%) | (polymer + | |
| | | | drug) | |
| 92 | Temsirolimus | PLGA 85/15 | 45.5% | NMP 54.5% |
| | (8.0%) | (37.5%) | (polymer + | |
| | | | drug) | |
| 93 | Temsirolimus | PLGA 50/50 | 49.0% | NMP 51.0% |
| | (9.0%) | (40.0%) | (polymer + | |
| | | | drug) | |
| 94 | Temsirolimus | PLGA 85/15 | 49.0% | NMP 51.0% |
| | (9.0%) | (40.0%) | (polymer + | |
| | | | drug) | |
| 95 | Temsirolimus | PLGA 50/50 | 49.0% | NMP 51.0% |
| | (9.0.%) | (40.0%) | (polymer + | |
| | | | drug) | |
| 96 | Temsirolimus | PLGA 85/15 | 49.0% | NMP 51.0% |
| | (9.0%) | (40.0%) | (polymer + | |
| | | | drug) | |
| 97 | Temsirolimus | PLGA 50/50 | 50.0% | NMP 50.0% |
| | (10.0%) | (40.0%) | (polymer + | |
| | | | drug) | |
| 98 | Temsirolimus | PLGA 85/15 | 50.0% | NMP 50.0% |
| | (10.0%) | (40.0%) | (polymer + | |
| | | | drug) | |
| 99 | Temsirolimus | PLGA 50/50 | 50.0% | NMP 50.0% |
| | (10.0%) | (40.0%) | (polymer + | |
| | | | drug) | |
| 100 | Temsirolimus | PLGA 85/15 | 50.0% | NMP 50.0% |
| | (10.0%) | (40.0%) | (polymer + | |
| | | | drug) | |
| 101 | Temsirolimus | PLGA 50/50 | 47.0% | NMP 53.0% |
| | (12.0%) | (35.0%) | (polymer + | |
| | | | drug) | |
| 102 | Temsirolimus | PLGA 85/15 | 47.0% | NMP 53.0% |
| | (12.0%) | (35.0%) | (polymer + | |
| | | | drug) | |

The SRF polymer concentration and molecular weight (MW) will influence the viscosity term in KIR. Values for polymer concentration and molecular weight outside of claimed ranges may fail KIR. For example, at high viscosity, the KIR value would be outside an acceptable range if the SRF failed because, for example, it was too viscous. TABLE 2 shows compositions which have a KIR value useful for treating BPH transurethrally, transperineally, or transrectally. Composition with KIR values that are not useful for treating BPH, include, but are not limited to, a SRF or composition comprising:
5% Drug; 60% Polymer; 35% NMP
7% Drug; 65% Polymer; 28% NMP
10% Drug; 60% Polymer; 30% NMP
12% Drug; 60% Polymer; 28% NMP.

Certain compositions with higher than 55% by weight bioabsorbable co-polymer, or lower than 25% by weight bioabsorbable co-polymer may have KIR values that are not useful for treating BPH.

In some embodiments, including any of the foregoing, the composition may include a bioabsorbable polymer at a concentration of 10-55%, 15-50% by wt.; and a drug at a concentration of 1%-20% by wt., or 1%-5% by wt. In certain embodiments, the composition also includes a solvent which makes up the remainder of the composition. In some embodiments, the solvent represents 20-60% by wt. of the composition. The % by wt. of polymer drug and solvent prescribe adds up to 100% wt. In some examples, the solvent is the majority species and the bioabsorbable polymer and drug are dissolved in the solvent. In some embodiments, including any of the foregoing, the solvent is a water-soluble solvent. In some embodiments, including any of the foregoing, the solvent is a water-soluble solvent capable of dissolving the drug and the bioabsorbable polymer. In certain embodiments, the drug and bioabsorbable polymer are present in the solvent above their saturation point. In some of these embodiments, the drug and bioabsorbable polymer may be present as a suspension in the solvent.

Number of injections/injection total volume. In a first pre-clinical canine study composition was delivered to the prostate by needle injection as a single, relatively large volume with some observed backflow through the needle. This approach resulted in injectate being diffused away from the target tissue, which is undesirable for reasons previously stated. In response, in the second pre-clinical canine study (below) the inventors instead tried a low injection volume of composition, minimized between 10-200 microliter per injection over 1-10 injections across each side of the prostate to reduce prostatic tissue pressure on the urethra to mitigate any potential backflow through the delivery device and/or loss of therapeutic injectate from the target tissue. A further injection volume range 50-100 microliter per injection over 1-5 injections across each side of the prostate was studied. The low unit volume (herein 0.05 ml to 0.2 ml) injections are designed to minimize swelling and bulking of the prostate.

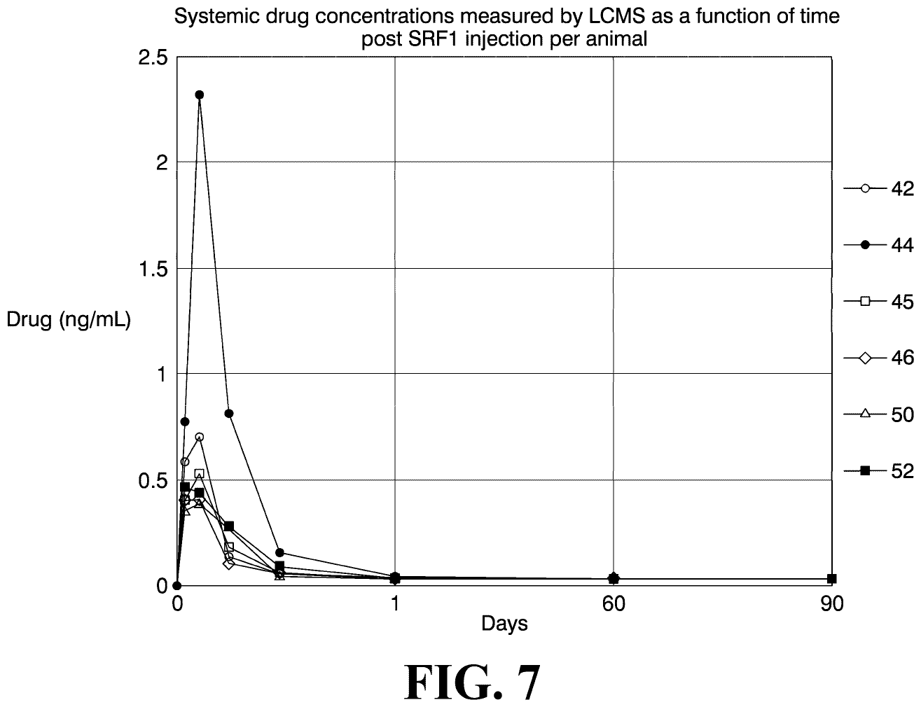

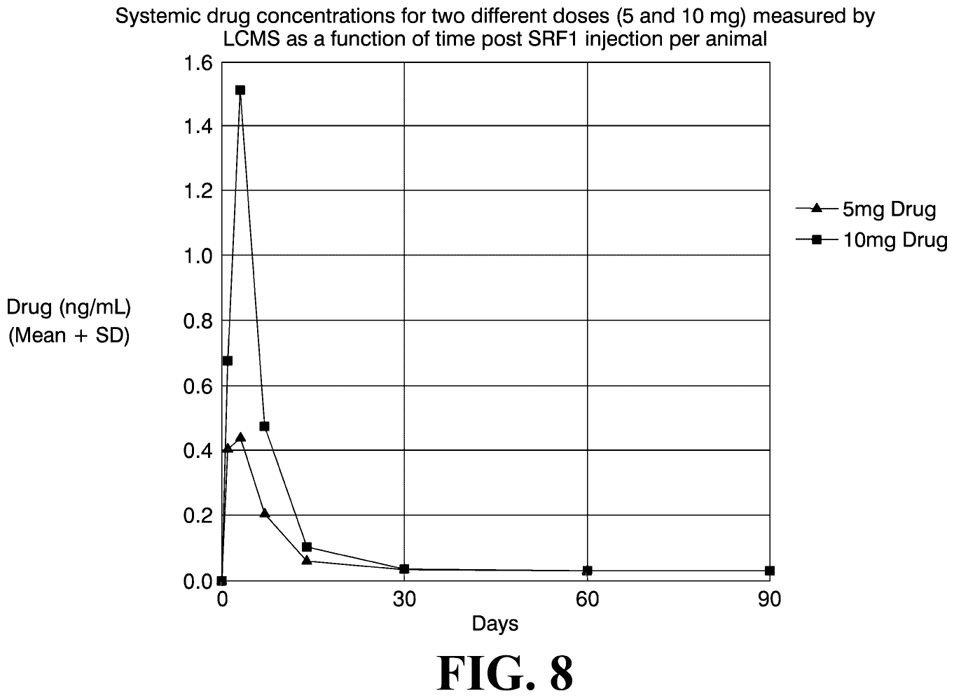

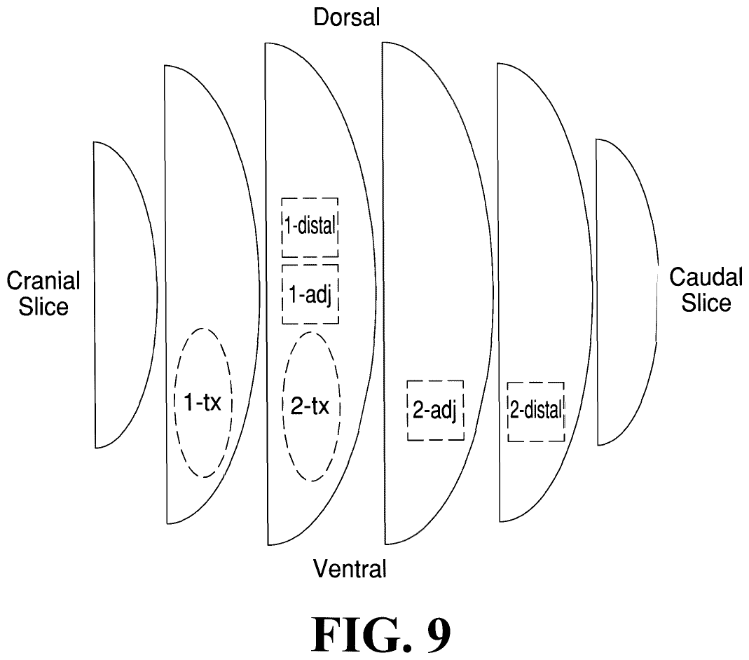

The distributed unit volume approach adopted (third characteristic) also produced an unexpected efficacious benefit. The second pre-clinical study, discussed infra., achieved a greater than 30% reduction in prostate size in the canine models. Notably, in the first study a higher volume of drug (by prostate weight) was used, compared to the total volume of drug in the second study. The SRF also was distributed throughout the prostate in unit volumes in the second study, but the total volume of drug (by prostate weight) in the second study was less than that used in the first study. This indicates that using smaller unit volumes distributed throughout the prostrate reduces prostate size more effectively than injecting a relatively higher unit volume at one location, which is a typical approach taken by others treating BPH. The distributed unit volume composition approach also showed controlled drug diffusion away (i.e., minimal drug lost to surrounding tissues and circulation) from the treatment area (see, FIG. 7 and FIG. 8 and accompanying text summarizing results).

In addition to the search for a composition that could satisfy the first two of the three characteristics sought by the inventors (minimal uncontrolled drug diffusion, sustained release rate and efficacy), there was the concomitant need to determine what kind of needle syringe could deliver the total volume of composition effectively, in a repeatable manner as one or more low unit volumes without the need for compromising the effectiveness of the SRF once injected. Thus, investigation into the deliverability of the composition to a patient was also needed.

A long, narrow needle (e.g., 20 cm with 0.06 cm lumen size for transrectal or 45 cm with 0.03 cm lumen size for transurethral) is desired as the manner for delivering the composition to the target tissue. Because it is a less invasive approach to accessing the target tissue than other methods for treating BPH and is more likely to be acceptable by patients in general. But use of this type of delivery device presented special challenges that needed to be overcome before the three characteristics of prostate treatment could be satisfied when using this delivery device. In one embodiment, the needle is a transurethral needle, discussed in greater detail below.

Delivery of a SRF, including a composition characterized by a relatively high viscosity for dispensing from a needle, is challenging when using a long, narrow needle of high slenderness and when dispensed in small volumes, due to the liquid shear stresses influenced by the needle inner surface area. The resistance to flow by a composition pushed through a long and narrow needle becomes more acute and deserving of attention the more viscous the liquid. Accordingly, for a relatively high viscosity fluid, which is more influenced by wall-fluid shearing stresses than a less viscous fluid, the inaccuracy in volume dispensed from a needle is more pronounced and varies proportionately with needle slenderness (defined as needle length, L, divided by lumen diameter, D). Accordingly, since the inaccuracy of volume delivered varies proportionately with LID and directly with the composition's viscosity, the smaller the dispensed volume desired and more viscous the composition, the more the inaccuracies are expected. The inventors, realizing this challenge regarding accurately dispensing the viscous composition for prostate treatment, had to identify both the embodiments of composition satisfying the first two requirements for KIR (i.e., the minimal uncontrolled drug diffusion, sustained release rate and efficacy requirements) and the needle syringe(s) that when used by a health professional enabled him/her to satisfy the third requirement for KIR (low unit volumes distributed over the prostate to attain the total volume of composition), without unduly compromising SRF effectiveness when introduced into the prostate.

On the one hand, bench tests indicated promising SRF formulations, but the corresponding compositions could not be reliably delivered using a long narrow needle. On the other hand, when the viscosity of the composition was reduced to make it easier to dispense the composition at the desired unit volume, either the desired drug release profile and/or gelation rate would not be achieved, or there would be an unacceptable level of drug diffusion into adjacent tissue or organs. Either the drug eluted too quickly from the SRF, the polymer gel was not dense or entangled enough, or the gelation rate was not high enough to avoid injectate flowing back to the needle and being drawn back along with the needle, away from the target tissue, after completion of the injection. Examples of compositions that failed to satisfy the KIR for these reasons were mentioned earlier.

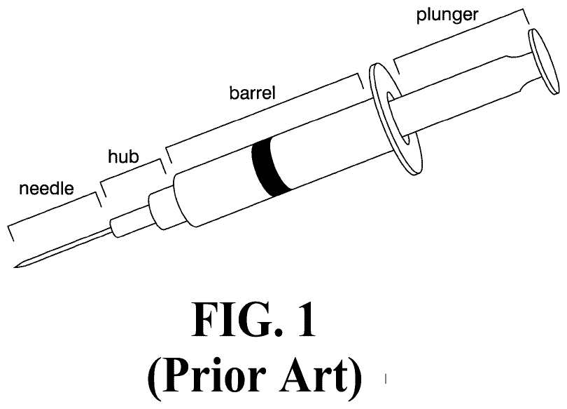

Thus, a careful balancing of constituents in the formulation—a polymer structure and molecular weight and concentration with a drug ratio and dose and solvent ratio to produce a desirable SRF with controlled release (e.g., as shown in FIG. 1, Table 1)—was eventually accomplished. But then it was found that the desired composition could not be delivered reliably as several unit volumes using a long needle as required to treat the prostate. Not infrequently, a more preferred, viscous formulation would need to be abandoned for a less viscous formulation to ensure that the right unit volume of composition with desired drug release could be repeatedly delivered using a long and narrow needle.

SRF1, which was used in the second pre-clinical study, discussed below, is an example of a SRF that satisfied the first two requirements for KIR (minimal uncontrolled drug diffusion, sustained release rate and efficacy) and was capable of being accurately and repeatedly delivered in low unit volumes, thereby also satisfying the third requirement for KIR. SRF1 is only but one of many examples disclosed herein that were found to satisfy the three requirements for KIR after conducting the validation, examination and testing described above (see FIGS. 14A, 14B and TABLES 3A, 3B for other examples of SRFs satisfying KIR requirements).

Two unit volumes (0.05 ml and 0.10 ml) were considered in the pre-clinical study. Both injection volumes showed significant reduction in prostate size, as reported in TABLES 5 and 6. Additionally, as shown in FIG. 7 and FIG. 8, there was very low systemic drug concentrations present in the blood, indicating that the agent was contained at the target tissue. The pre-clinical study therefore indicates that an SRF injectate according to the disclosure can (1) reduce prostate volume over a 30 and 90 day period, and (2) limit substantially all of the active agent to only the target tissue. Moreover, the study indicated unexpectedly a significant ratio of prostate 30-day and 90-day drug concentration to maximum plasma drug concentration of at least 10,000. A unit volume of SRF injectate needed for efficacy was also demonstrated. SRF (SRF1) injectate was minimized to between 10-200 microliter per injection over 1-10 injections across each side of the prostate, or 50-100 microliter per injection over 1-5 injections across each side of the prostate.

As for compositions comprising the SRF, drug and polymer concentrations in the composition may range from 0.1 wt % up to 60 wt %. Injection volumes of the SRF may range from 25 microliters up to 5 mL per injection. Overall dosage of drug provided in the SRF can range from 50 μg up to 200 mg. For example, a composition comprises 300 μg of sirolimus dissolved with 3,000 μg (“μg”—micrograms) poly (D, L-lactide-co-glycolide) (85:15) in NMP at an about 50 solid wt. % concentration.

In some embodiments, including any of the foregoing, a composition comprises 300 μg of paclitaxel dissolved with 3,000 μg (“μg”—micrograms) poly (D, L-lactide-co-glycolide) (85:15) in NMP at an about 50 solid wt. % concentration. In some embodiments, including any of the foregoing, a composition comprises 500 μg of sirolimus dissolved with 2,900 μg (“μg”—micrograms) poly (D,L-lactide-co-glycolide) (85:15) in NMP at an about 50 solid wt. % concentration. In some embodiments, including any of the foregoing, a composition comprises 500 μg of paclitaxel dissolved with 2,900 μg (“μg”—micrograms) poly (D, L-lactide-co-glycolide) (85:15) in NMP at an about 50 solid wt. % concentration. In some embodiments, including any of the foregoing, a composition comprises 300 μg of sirolimus dissolved with 3,000 μg (“μg”—micrograms) poly (D, L-lactide-co-glycolide) (50:50) in NMP at an about 50 solid wt. % concentration. In some embodiments, including any of the foregoing, a composition comprises 300 μg of paclitaxel dissolved with 3,000 μg (“μg”—micrograms) poly (D, L-lactide-co-glycolide) (50:50) in NMP at an about 50 solid wt. % concentration. In some embodiments, including any of the foregoing, a composition comprises 500 μg of sirolimus dissolved with 2,900 μg (“μg”—micrograms) poly (D, L-lactide-co-glycolide) (50:50) in NMP at an about 50 solid wt. % concentration. In some embodiments, including any of the foregoing, a composition comprises 500 μg of paclitaxel dissolved with 2,900 μg (“μg”—micrograms) poly (D, L-lactide-co-glycolide) (50:50) in NMP at an about 50 solid wt. % concentration. As used herein, the term solvent refers to a solvent capable of fully or homogenously dissolving substances added to the solvent.

Further examples of compositions that satisfy the first two characteristics (efficacy and sustained release, and low diffusion) are provided in the examples, below.

Precise delivery of low volumes of a SRF for therapeutic retention with a precise drug dosage may be accomplished using a 250-500, or 250-2500 microliter or smaller needle syringe. This delivery device may have a gastight, glass cyclic olefin polymer, or polypropylene syringe body with Teflon, polyethylene, cyclic olefin polymer, or polypropylene plunger connected with a 20 cm long 20 g needle, which has a 0.06 cm lumen diameter. Herein, 20 g is 20 gauge. The volume of the delivery syringe must be large enough to accommodate the dead volume within the needle of approximately 150 microliter. Therefore, a 250 microliter gastight syringe can be utilized to deliver one to two 50 microliter unit volumes of a composition. For multiple injections from the same delivery device a 250-1,000 microliter gastight syringe volume may be used, which allows sufficient volume to overcome needle dead volume and can deliver at least eight 100 microliter unit volume injections to each lobe of a prostate.

Alternatively, a 1,000 microliter or 1 milliliter cyclic olefin polymer and/or polypropylene syringes may be utilized. For example, the SRF may be provided as a kit containing one syringe with the PLGA dissolved in NMP solvent with female luer lock and the other syringe with the drug powder with male luer lock. These syringes are then directly connected and mixed back and forth a number of times (fifty) in order to completely solubilize the drug such that no drug particles remain. A lower number of mixes (ten) can also be utilized to result in a partial solution partially mixed SRF with remaining drug particulate that can produce an extended drug release time in vivo due to the additional requirement of drug particle dissolution in the SRF. After mixing the SRF is loaded into the graduated syringe for delivery via long slender needle.

Precise delivery of low volumes of a SRF for therapeutic retention with a precise drug dosage may also be accomplished using a 2,000 microliter or 2 milliliter syringe. This delivery device may have a gastight, glass cyclic olefin polymer, or polypropylene syringe body with Teflon, polyethylene, cyclic olefin polymer, or polypropylene plunger connected with a 45 cm long 23 g needle, which has a 0.03 cm lumen diameter. The volume of the delivery syringe must be large enough to accommodate the dead volume within the needle of approximately 1.26 milliliter. Therefore, a 1,500 microliter gastight syringe can be utilized to deliver one to two 50 microliter unit volumes of a composition. For multiple injections from the same delivery device a 2,000-3,000 microliter gastight syringe volume may be used, which allows sufficient volume to overcome needle dead volume and can deliver at least eight 100 microliter unit volume injections to each lobe of a prostate. Alternatively, a 3,000 microliter or 3 milliliter cyclic olefin polymer and/or polypropylene syringes may be utilized.

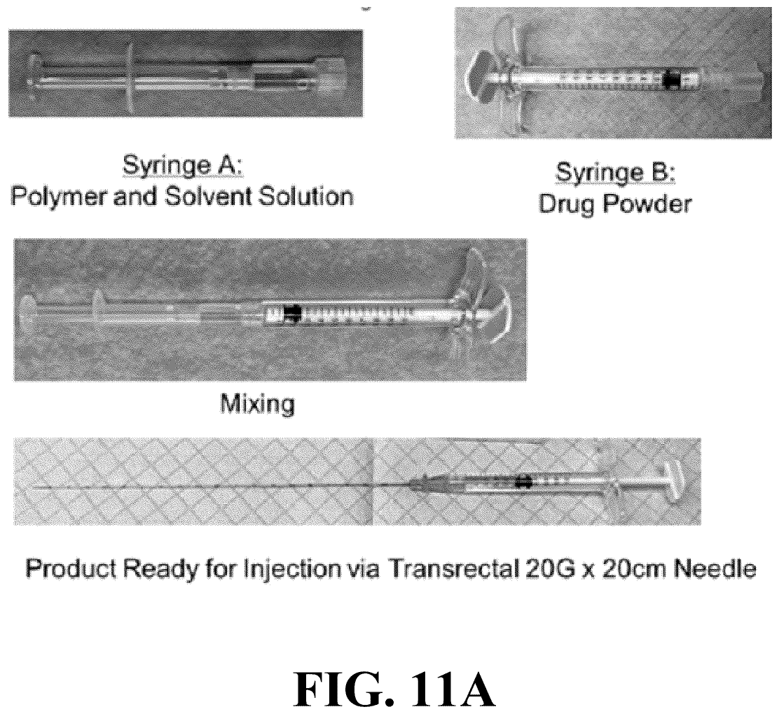

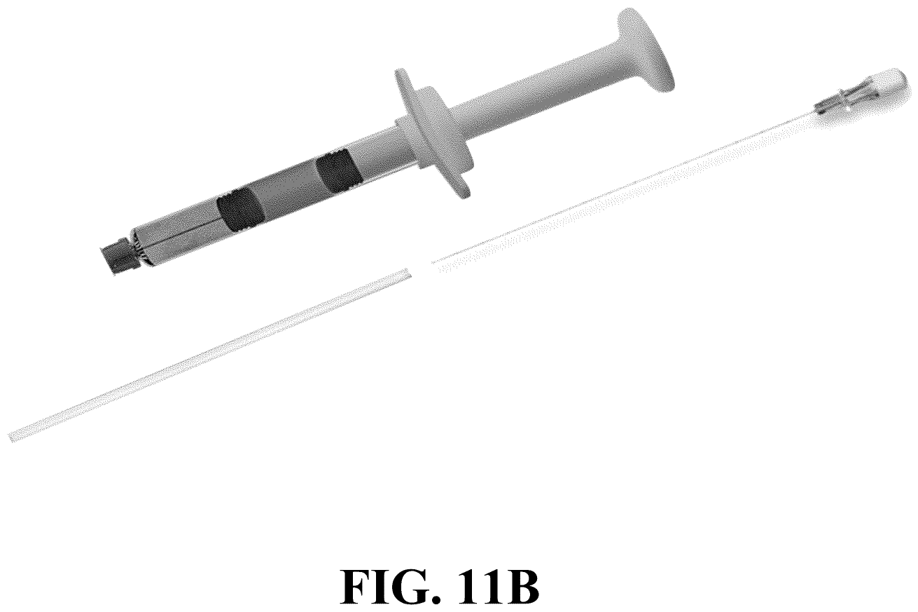

For example, the SRF may be provided as a kit containing one syringe with the PLGA dissolved in NMP solvent with female luer lock and the other syringe with the drug powder with male luer lock. These syringes are then directly connected and mixed back and forth a number of times (fifty) in order to completely solubilize the drug such that no drug particles remain. A lower number of mixes (ten) can also be utilized to result in a partially mixed SRF with remaining drug particulate that can produce an extended drug release time in vivo due to the additional requirement of drug particle dissolution in the SRF. After mixing the SRF is loaded into the graduated syringe for delivery via long slender needle. An example embodiment is shown in FIG. 11A and FIG. 11B.

FIG. 11A and FIG. 11B show embodiments of syringes that would, in some embodiments, be included in a kit disclosed herein.

See, for example, FIG. 11A. FIG. 11A shows Syringe A, which includes a solution of the bioabsorbable polymer, PLGA, in NMP solvent. FIG. 11A also shows Syringe B, which includes a drug powder. Above the label “Mixing,” FIG. 11A shows Syringe A and Syringe B connected via the female luer lock on Syringe A. In the “Mixing” configuration, in which the two syringes are connected via a luer lock, the solution of the bioabsorbable polymer, PLGA, in NMP solvent can be mixed with the drug powder to form a second solution in which the drug and the bioabsorbable polymer are both dissolved. This second solution is shown at the bottom of FIG. 11A above the label stating “Product Ready for Injection via Transrectal 20G×20 cm Needle.”

FIG. 11B shows a syringe barrel having two chambers separated by a septum. One of these two chambers may include a solution of the bioabsorbable polymer, PLGA, in NMP solvent. The other of these two chambers may include drug powder. When the separating septum is breached or punctured, the contents of the two chambers can mix to form a second solution in which the drug and the bioabsorbable polymer are both dissolved. FIG. 11B also shows a transurethral needle and needle cover.

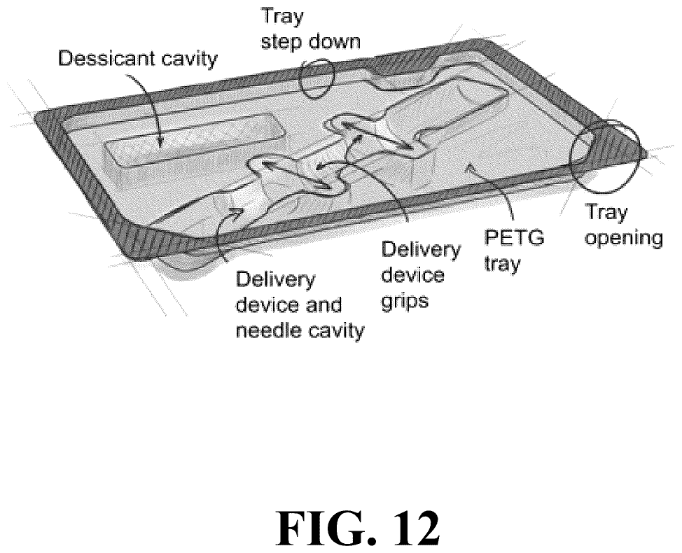

See, also FIG. 12, which shows an embodiment of a kit disclosed herein. FIG. 12 shows a pre-formed, sterilized PETG tray with impressions for holding, inter alia, kit components. For example, FIG. 12 shows a Desiccant cavity for holding a desiccant and/or oxygen absorber. FIG. 12 shows a Delivery device and needle cavity for holding the same. The delivery device and needle cavity include space to accommodate the delivery device finger rests. FIG. 12 shows the PETG tray as having a tray step down and Tray openings.

Referring to FIG. 1, a typical needle syringe includes an applicator portion (plunger, barrel), and hub portion that connects the needle with the interior chamber of the barrel and forms a fluid-tight connection for passage of a fluid contained within the barrel through the needle lumen.

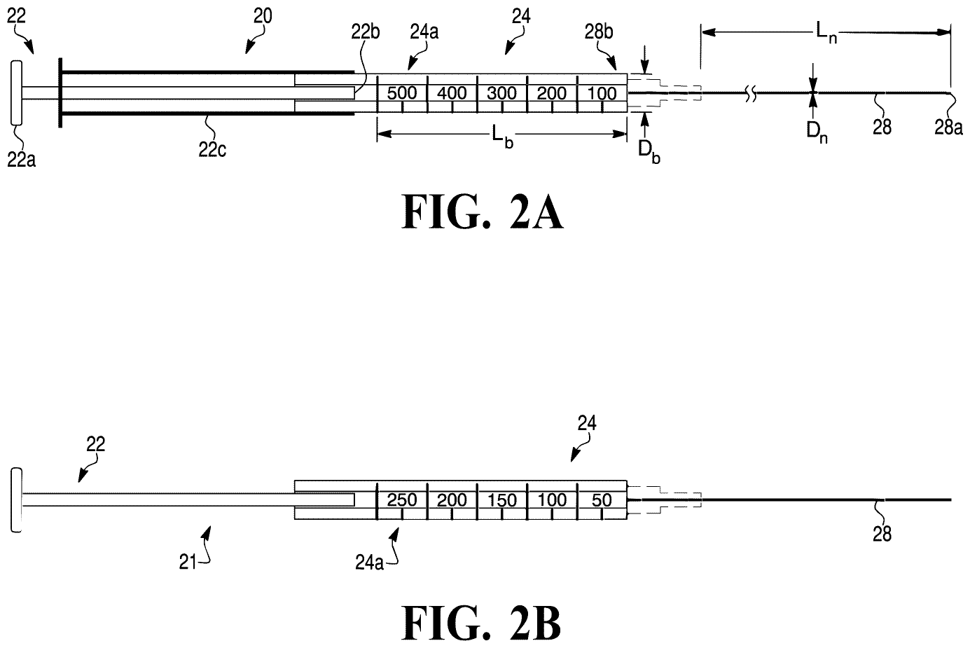

Referring to FIGS. 2A and 2B, there is shown side view schematics of needle syringes 20, 21 according to the disclosure. More specifically, the needle syringes depicted have sizes for holding and dispensing a unit volume through a long, narrow needle tip 28a. The needle 28 is characterized by a length Lₐ and lumen diameter Dₙ (a needle hub is shown in phantom for the sake of illustrating the measured length Lₙ, which is taken from the upstream opening 28b forming the fluid-tight connection between the barrel and needle 28 body and the terminal, or downstream-most tip that first punctures the skin). The needle may be made from stainless steel.

The needle syringe in FIG. 2A shows by example graduations of 500/400/300/200/100/50/25 micro-liters for dispensing unit volumes of e.g., 0.05 ml (50 micro-liters). The barrel capacity is 1000 micro-liters. The barrel 24 has a length L_{b} and barrel inner or lumen diameter D_{b}. The prefilled syringe barrel (1000 micro-liter) may have attached to it a needle length Lₐ of 20 cm and the needle may have a 150-200 micro-liter dead volume. The syringe, which may need to be prefilled, may also incorporate a stabilizer 22c for guiding the plunger head 22b towards the needle opening 28b. The composition comprising the SRF (contained in the syringe barrel 24 and in amounts up to about 500 micro-liters) is ejected through the needle tip 28a by finger pressure applied to the plunger head 22a. In this example, the barrel, when holding 500 micro-liters of composition can hold, for example, up to 7, 50 micro-liter unit volumes accounting for the 150 micro-liter dead volume. The needle syringe in FIG. 2B is the same in several respects as the syringe in FIG. 2A, except that it has instead a 3000 micro-liter barrel capacity. It may also incorporate a stabilizer 22c. In another example a 3000 micro-liter capacity syringe has graduations of 200 Ul, 100 Ul, and 50 Ul to help with more accurately dispensing low unit volumes of the composition.

The needle syringe of FIG. 2A may be provided with features or an additional structure that allows the needle syringe to provide incremental dosages of a composition contained therein. According to one example, the incremental dosages correspond to a single unit volume or incremental fractions of a unit volume (e.g., ½ of a unit volume, ¼ of a unit volume, etc.) According to one example, the additional structure may be a dosing guide. The dosing guide may be made from plastic or any other desired material and may be economically manufactured using injection molding or any other desired process. The dosing guide may be easily attachable and removable from the needle syringe and adaptable to needle syringes of various dimensions.

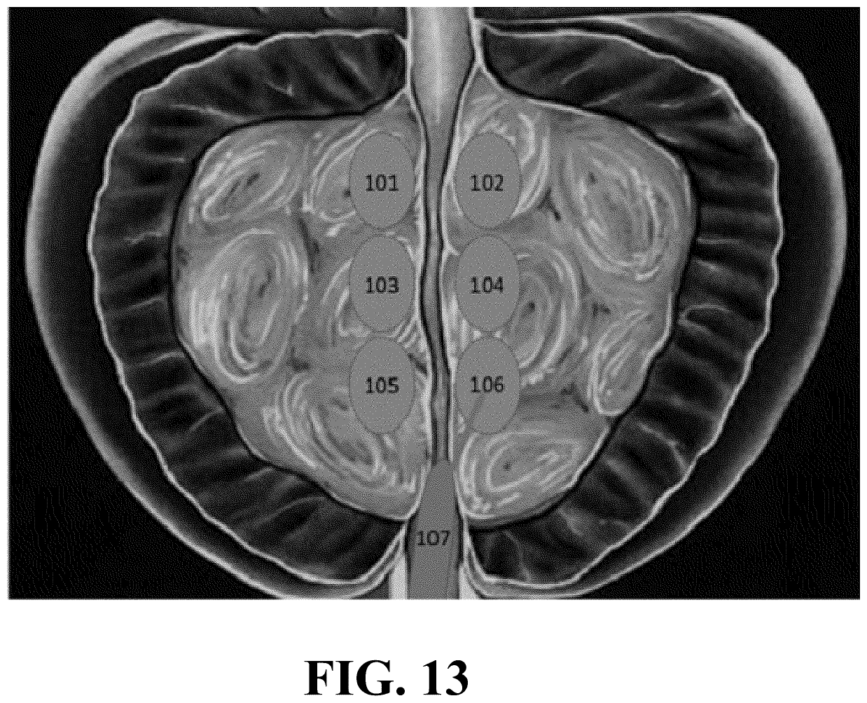

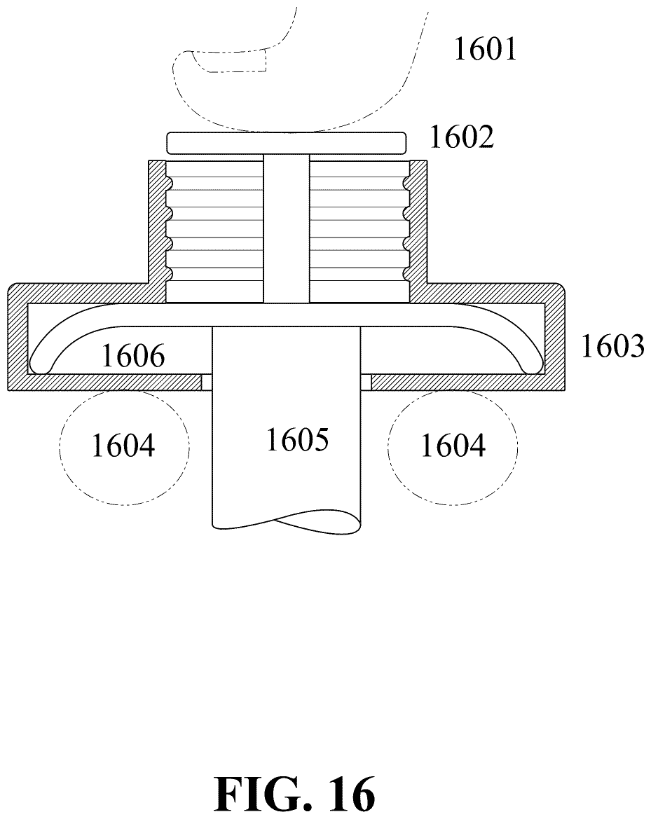

FIG. 13 shows an illustration of a prostate with a urethra running through its center. In FIG. 13, a needle passes through and exist from the end of a catheter 107 at an angle to inject a unit volume of composition at location 106. The needle end has a depth limiter associated with it. As this needle moves through the urethra, the needle injects a unit volume, v, of a composition comprising an SRF at locations 101, 102, 103, 104, 105, and 106, which number of injection sites is based on the length of the transition zone urethral length (TZUL). The needle has a retractable tip that penetrates the tissue at positions 101, 102, 103, 104, 105, or 106 to deliver the unit volume at each of these positions. The unit volume delivery may be assisted by using a dose guide attached to the syringe, such as the dose guide shown in FIG. 16. The number of injection sites based on the TZUL is provided in TABLE 1B. It was found that the number of injections n is related to TZUL as n=TZUL/(11*v). FIG. 13 depicts the n=6 delivery of the composition comprising an SRF at 6 different locations along the length of the urethra having a length TZUL. Each oval represents drug tissue exposure from a unit volume treatment.

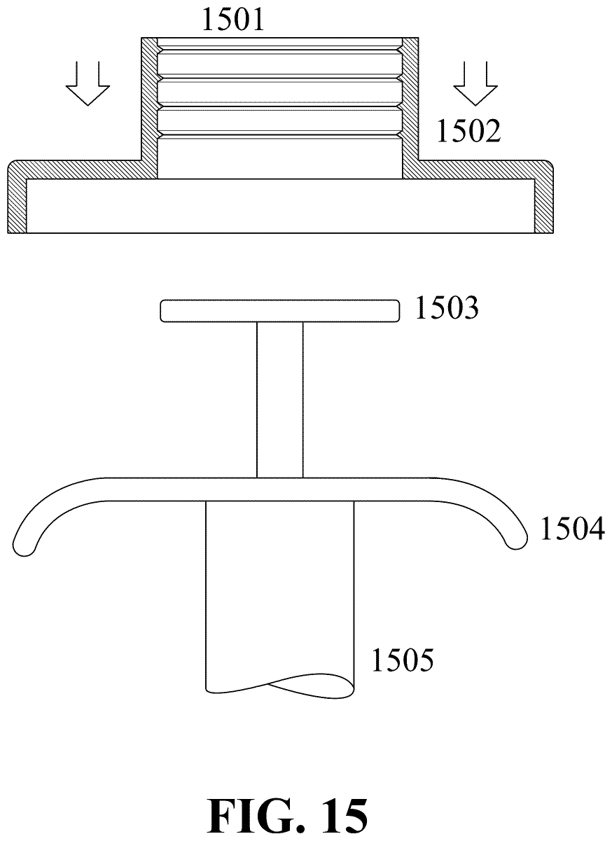

FIG. 15 shows an embodiment of a dosing guide 1502 prior to installation on a needle syringe. The dosing guide 1502 may have four (or more) detents (1501) on the interior wall. Embodiments are contemplated herein that include more or less detents. As will be explained herein, a distance between the detents may correspond to a single unit volume or incremental fractions of a unit volume. The dosing guide 1502 is open on the bottom so that when dosing guide 1502 engages the syringe plunger and barrel shown below the dosing guide, the dosing guide rests on top of the finger rest 1504. Also shown is plunger 1503 and barrel 1505. In this embodiment, when the needle syringe is coupled to the dosing guide 1502, an operator can engage the bottom of the finger rest 1504.

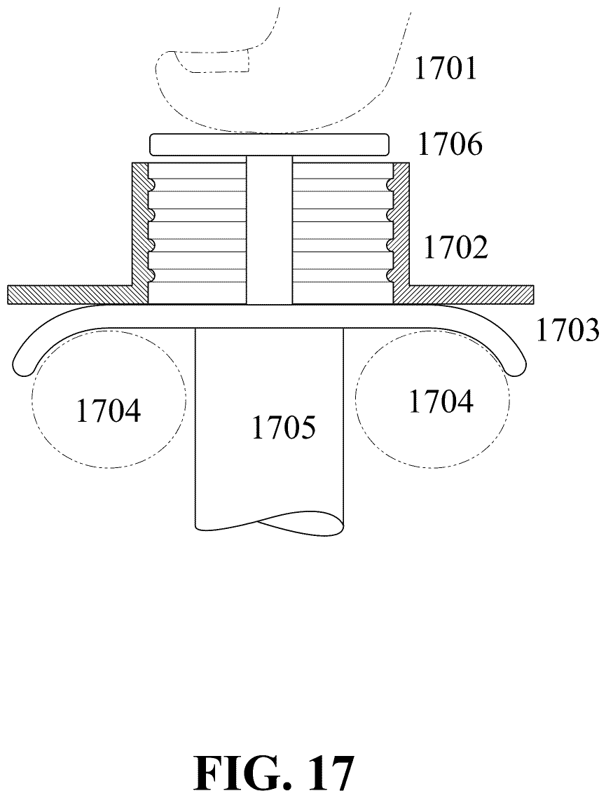

FIG. 16 shows an embodiment of a dosing guide 1603 that surrounds the finger rests 1606. Also shown is plunger 1602, which is being depressed by a thumb 1601, and barrel 1605. Positions 1604 represent pointer finger and index finger positions for creating opposing force with the thumb 1601. In this embodiment, when the needle syringe is coupled to the dosing guide 1603, an operator does not engage the bottom of the finger rest 1606 directly. Instead, as shown in FIG. 16, the operator engages a surface of the dosing guide 1603. The finger rest is contained within. This arrangement may be preferred as it offers a more secure hold of the guide to the top of the syringe, as compared to the arrangement in FIG. 17. When fingers 1604 are being pressed into the guide (in response to the thumb pushing down on the plunger head) the guide is held firmly in place on the syringe. The guide is therefore less likely to move about because fingers 1604 are pressing the guide sleeve against the syringe wings, as shown, during use.

FIG. 16 shows the guide wings fully enclosing the syringe wings or finger rests. In an alternative embodiment the syringe wings may extend out partially from the guide wings. The partial enclosure embodiment would be the same as shown (finger 1604 presses guide into syringe wing) except that left and right outer ends of the syringe wings extend outwardly from openings provided in the respective left and right guide wings

FIGS. 17 and 19-23, herein, show other embodiments of a dosing guide.

FIG. 17 shows an embodiment of a dosing guide 1702 that sits atop the finger rests 1703. The embodiment shown in FIG. 17 is similar to the embodiment shown in FIG. 15 except for the omission of downwardly extending wings on the part of the dosing guide 1702 that sits atop the finger rests 1703. Also shown is plunger 1706, which is depressed by a thumb 1701, and barrel 1705. Positions 1704 represent pointer finger and index finger positions for creating opposing force with the thumb 1601.

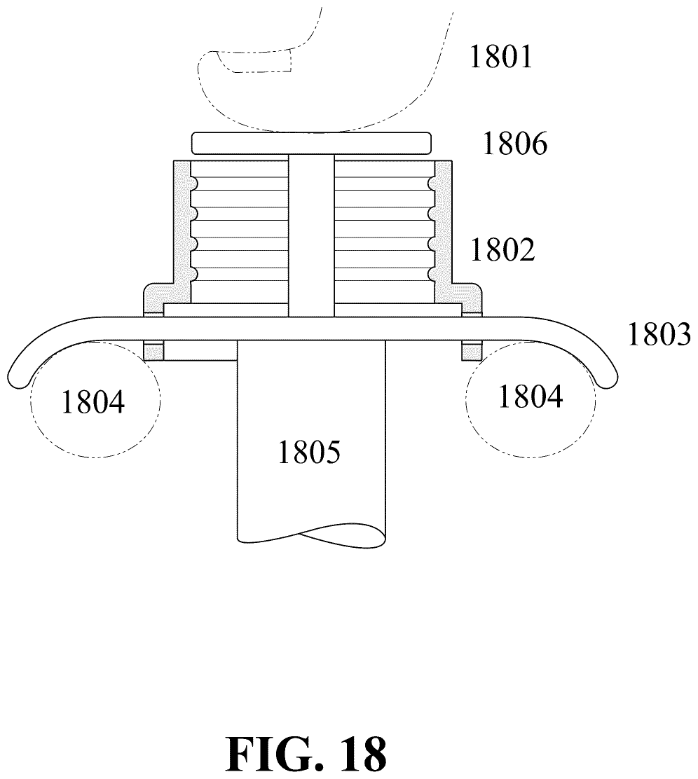

FIG. 18 shows an embodiment of a dosing guide 1802 that attaches atop and below the finger rests 1803. In this embodiment the finger rests 1803 extend through openings provided on a body of the dosing guide 1802. Also shown is plunger 1806, which is being depressed by a thumb 1801, and barrel 1805. Positions 1804 represent pointer finger and index finger positions for creating opposing force with the thumb 1801.

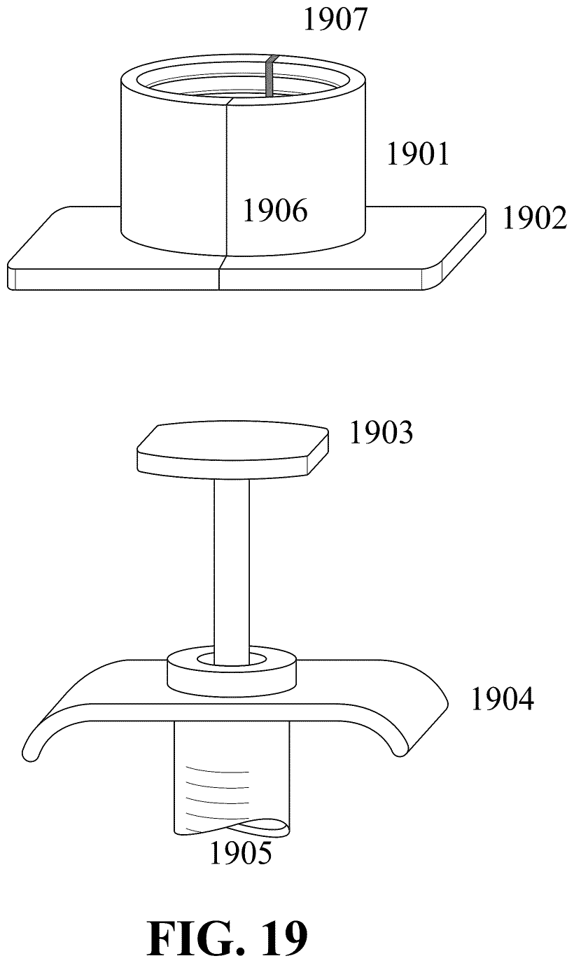

FIG. 19 shows an embodiment of a dosing guide similar to the dosing guide of FIG. 15, wherein when the needle syringe is coupled to the dosing guide, an operator can directly engage the bottom of the finger rests. As shown in FIG. 19, the dosing guide has a top portion 1901 that can surround the plunger 1903 when bottom portion 1902 sits atop the finger rests 1904. Also shown is barrel 1905. Also shown is the opening 1906 with a hinge 1907. The opening 1906 separates the dosing guide into a first halve and a second halve that are connected by the hinge 1907. The hinge 1907 (e.g., a living hinge) allows the first part and the second part to move relative to one another, in a clamshell-type arrangement. This relative movement facilitates attachment of the dosing guide to a needle syringe, as well as removal of the dosing guide from the needle syringe. According to this embodiment, the first part and the second part can move relative to one another about the hinge 1907, thereby creating an opening to receive or remove the needle syringe from the dosing guide.

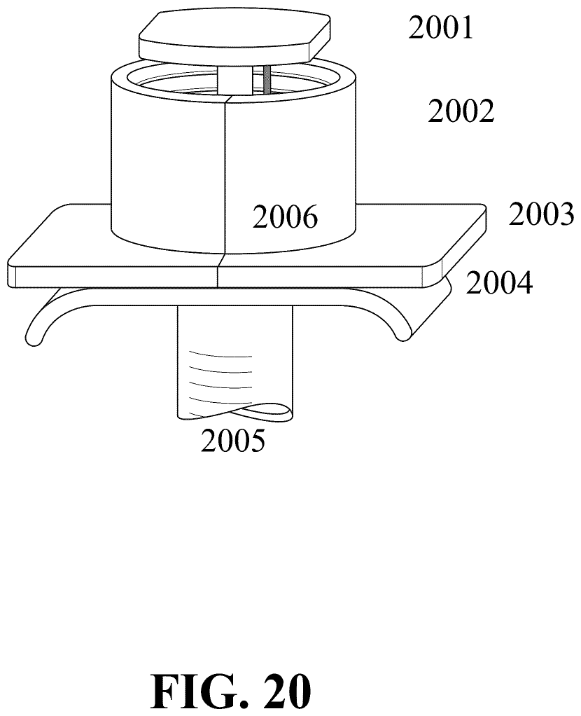

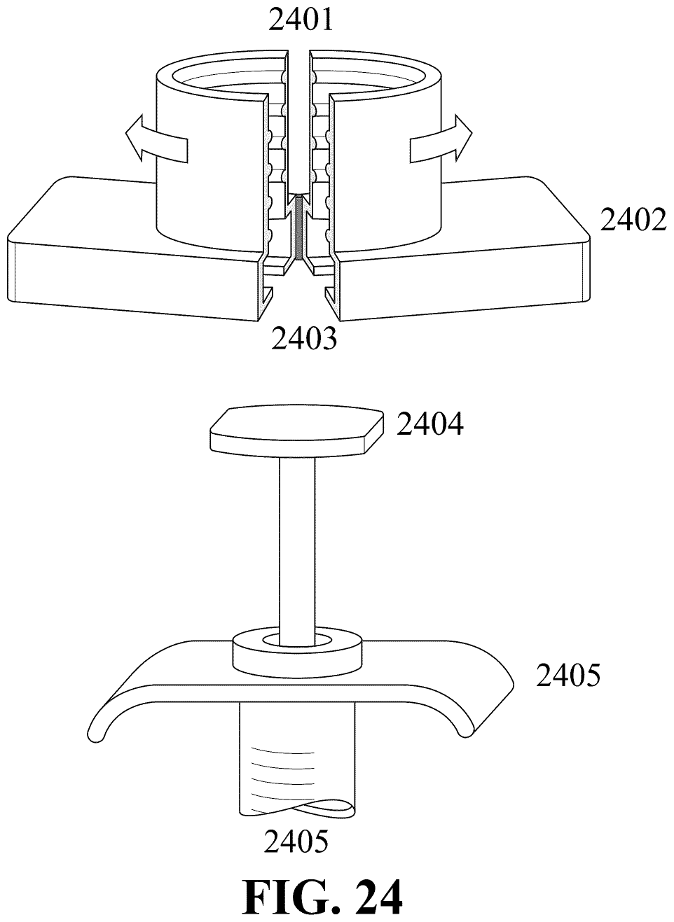

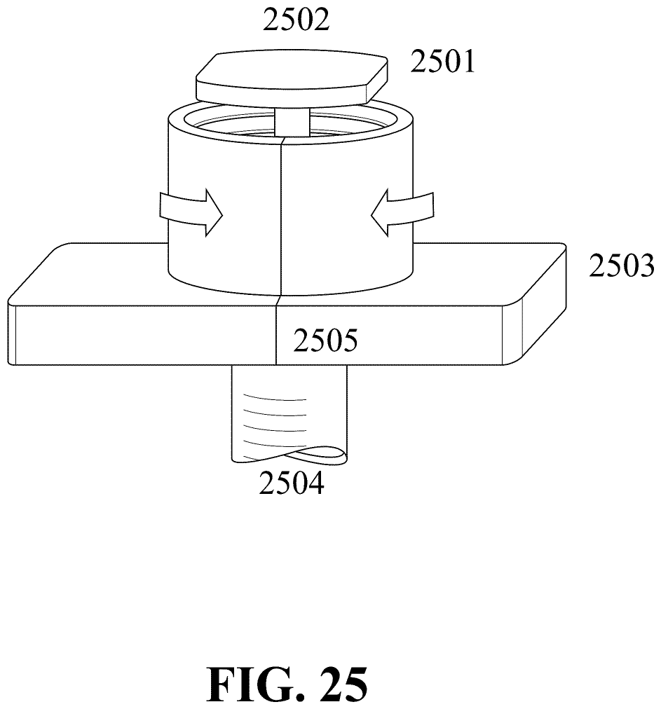

FIG. 20 shows the embodiment of FIG. 19 after the dosing guide receives the needle syringe. As shown, the dosing guide has a top portion 2002 that surrounds the plunger 2001 when bottom portion 2003 sits atop the finger rests 2004. Also shown is barrel 2005. Also shown is barrel 1905. Also shown is the opening 2006 with a hinge (not labeled) directly behind the opening 2306. According to one embodiment, the needle syringe is primed before being received by the dosing guide. In other words, a dead volume of the needle syringe is expelled from the barrel 1905 before the dosing guide receives the needle syringe. After the needle is primed, the syringe is enclosed within the dosing guide as shown in FIG. 24 and FIG. 25. dosing guide as This calibrates the plunger head position with respect to the detents in the guide top portion, so that the needle syringe dispenses the desired unit volume amounts, or incremental fraction of the unit volume as the plunger 2001 is advances between the spaced apart detents. According to one embodiment, the dosing guide is dimensioned and configured so that the plunger 2001 is located adjacent to a terminal end of the top portion 2002 when the needle syringe is primed and received in the dosing guide. In other embodiments, the dosing guide is dimensioned and configured so that the plunger 2001 is engaged with a terminal end of the top portion 2002 when the needle syringe is primed and received in the dosing guide. It will be appreciated that in general, the plunger head position will not be resting directly against the uppermost detent of the guide when primed. In these cases the medical professional will need to expel some additional composition until the head rests against, or near to the top detent. This will ensure that when the plunger head advances past the top detent, and rests between it and the next detent, the desired unit volume will have been dispensed.

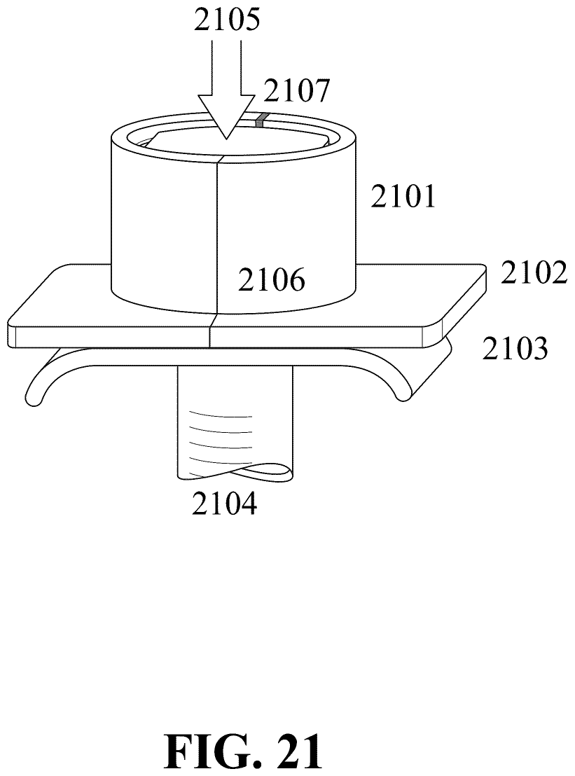

FIG. 21 shows the embodiment of FIG. 19 after the needle syringe is received in the dosing guide and the plunger has been depressed. As shown, the dosing guide has a top portion 2101 that surrounds the plunger 2005, which is depressed, and bottom portion 2102, which sits atop the finger rests 2103. Also shown is barrel 2104. Also shown is barrel 1905. Also shown is the opening 2106 with a hinge 2107. As shown in FIG. 21, depressing the plunger 2005 causes the plunger 2005 to move into an interior space of the top portion 2101.

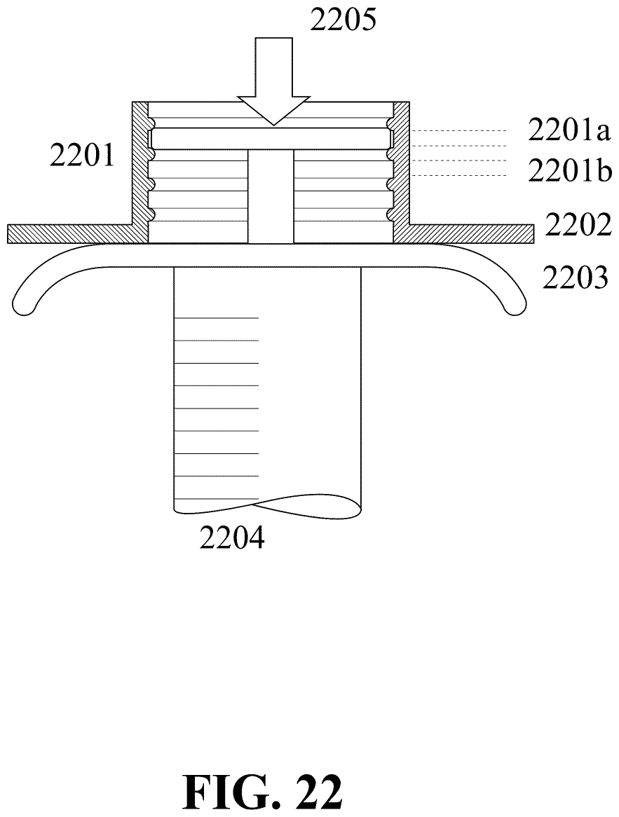

FIG. 22 shows a sectional view of FIG. 21 after the plunger is further depressed. As shown, the dosing guide has a top portion 2201 that surrounds the plunger 2205, which is depressed past a first detent, and bottom portion 2202, which sits atop the finger rests 2203. Also shown is barrel 2104. In FIG. 22, the detents are equally spaced from each other. According to one embodiment, a distance between one detent 2201(a) and an adjacent detent 2201(b) corresponds to a single unit volume of the needle syringe contained therein. In alternative embodiments, the distance between adjacent detents 2201(a), 2201(b) corresponds to incremental fractions of a unit volume (e.g., ½ of a unit volume, ¼ of a unit volume, etc.).

The detents are 2201 are configured to engage the plunger 2205 as the plunger 2205 is moved. This engagement resists further movement of the plunger 2205, thus facilitating the administration of unit volumes or increments thereof. A user can overcome this resistance to move the plunger 2205 past one detent and into engagement with another adjacent detent, thereby administrating another unit volume. The detents and radial flexing of the cylinder (in response to plunger movement) may be such as to promote a convenient and easily sensed haptic or audible feedback to the medical professional indicating that the plunger has moved between detents. The relationship between movement of the plunger head between detents and the volume of composition dispensed will depend on the inner diameter of syringe being used. Differently sized dose guides may be provided and identify corresponding volumes dispensed using different sized syringes. The relationship between the spacing of detents 2201a and 2201b, that is, the distance LDC, and a unit volume, v, and the inner diameter of the syringe, d, is LDC=[4/(πd²)]v. Detents spaced by the vertical distance LDC (in the direction of plunger movement) are related to the unit volume v dispensed using a syringe with diameter d when the plunger head is advanced past a detent.

The term “detent” may refer to any of the following surfaces formed on the inner walls of the top portion: a rim, lip or ridge extending at least partially around the inner wall (e.g., continuous, or discontinuous and spanning 90, 180, 270 or 60 degree sections of the cylinder wall), semicircular bumps that are spaced apart circumferentially by 20, 30, 45 or 60 degrees from each other. In each of these examples, the vertical spacing (i.e., in the direction of plunger travel) between detents is registered to the inner diameter of the syringe, as explained further below, so that when the plunger moves from one detent to another the syringe will dispense a unit volume.

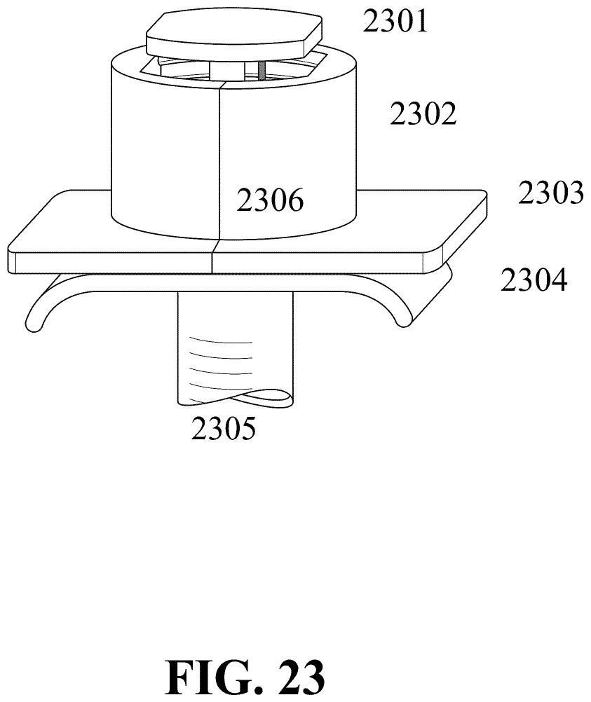

It is desired that the detents impose some resistance to the plunger head, but only enough as needed to produce the haptic or audible feedback. If the resistance is too high, then this will interfere with the dispensing of the unit volumes and ease of use. To ensure the resistance is not too high, vertical slits or notches may be formed in the top portion to reduce the radial or hoop stiffness in the top portion as a unitary cylinder. For instance, referring to FIG. 23, each half of the top portions of the clamshell that form the cylindrical body may instead be two sections extending up from the guide's wing portions, each section forming a 90 degree section of the cylinder shown. These half cylinder sections may alternatively be formed by three 60 degree sections, which should provide for an even more flexible top portion in response to plunger movement while still providing the desired haptic and audible feedback to the medical professional. Rather than being separate parts of the cylinder as just described, the sections may instead correspond to vertical notches.

FIG. 23 shows a variant of the embodiment shown in FIG. 20. As shown, the dosing guide has a top portion 2302 that surrounds the plunger 2301 when bottom portion 2303 sits atop the finger rests 2304. Also shown is barrel 2305. Also shown is the opening 2306 with a hinge (not labeled) directly behind the opening 2306. In FIG. 23, the opening defined by the top portion 2302 has a substantially obround configuration, in comparison to the arrangement shown in FIG. 20, wherein the opening defined by the top portion 2302 has a substantially circular configuration. Additionally, in FIG. 23, the detents are provided on only part of the interior wall (i.e., are discontinuous), in comparison to the configuration shown in FIG. 20, wherein the detents extend continuously around the interior wall. In alternative embodiments, the opening defined by the top portion may have any desired configuration and may be selected to mimic the shape of the plunger head. In other alternative embodiments, the detents may extend around any percentage of the interior wall and may be provided in a continuous or discontinuous manner, as mentioned earlier.

FIG. 24 shows an embodiment of a dosing guide 2402 with a living a hinge 2401 such that the dosing guide can open at position 2403 like the clam shell, as mentioned above. This hinge 2401 allows the dosing guide 2402 to engage with the plunger 2404 and finger rests 2405. Desirably one may prefer the front surface of the guide wing shown to be instead open so that the syringe wing may be more easily fit within the dose guide when the clamshell is closed around the syringe wings. Also shown is barrel 2405. Similar to the embodiment shown in FIG. 19, the hinge 2401 allows parts of the dosing guide 2402 to move relative to one another, which facilitates attaching the dosing guide 2402 to the needle syringe, and removal from the needle syringe.

FIG. 25 shows the embodiment of the dosing guide of FIG. 24 after the two parts of the dosing guide have been moved to enclose the needle syringe. As shown, the dosing guide 2503 has a closed hinge 2502 and the dosing guide is closed at position 2505. Also shown is plunger 2501 and barrel 2504.

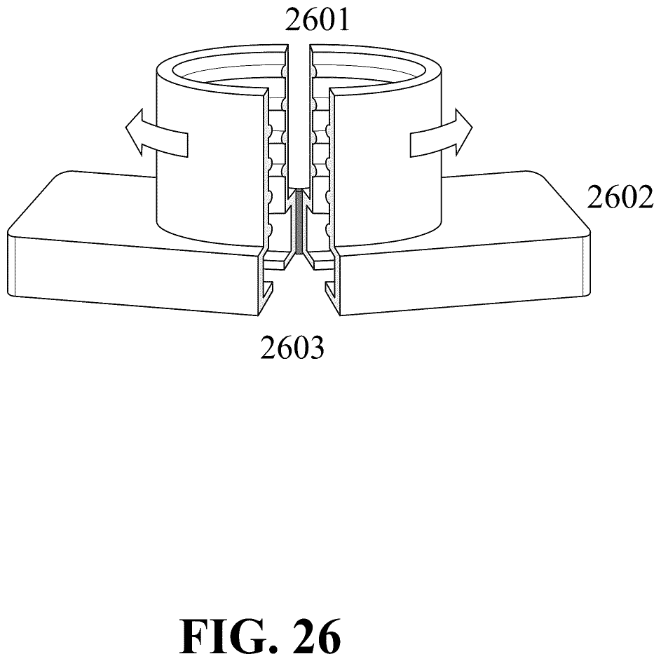

FIG. 26 shows how the embodiment of the dosing guide of FIG. 24 can be manipulated to facilitate attachment or removal of the dosing guide. As shown, the dosing guide 2602 has a hinge 2601 such that the dosing guide can open at position 2603 like a clam shell. The arrows show how the hinge 2601 allows for movement of the parts of the dosing guide 2602. Specifically, the arrows show how the parts of the dosing guide 2602 may be moved relative to one another to create an opening to receive or remove the needle syringe from the dosing guide.

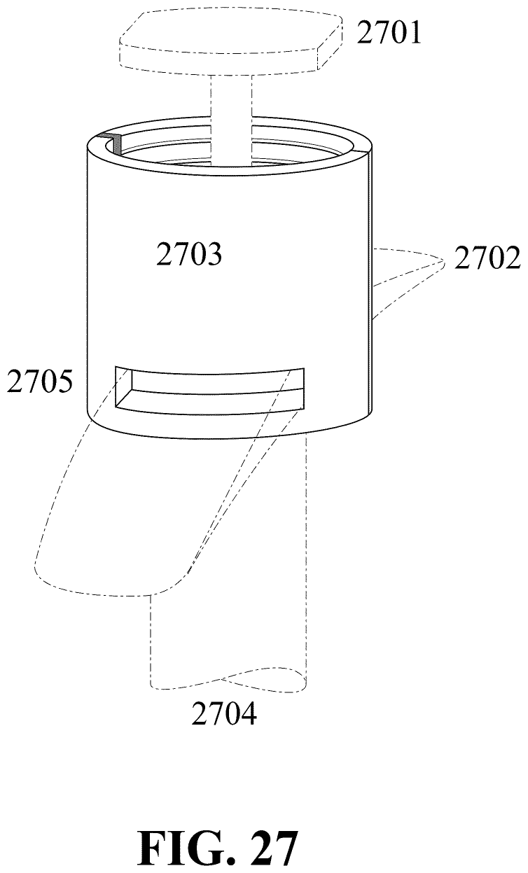

FIG. 27 shows another embodiment of a dosing guide 2703 in use with a needle syringe. The needle syringe includes a plunger 2701, finger rests 2702, and barrel 2704. The barrel 2704 extends from the bottom of the dosing guide 2703. The dosing guide includes apertures 2705 (i.e., cut-out opening). When the needle syringe is received in the dosing guide 2703 the finger rests 2702 extend through the apertures 2705. The position of the finger rests 2702 within the apertures 2705 prevents relative movement between the dosing guide 2703 and the needle syringe.

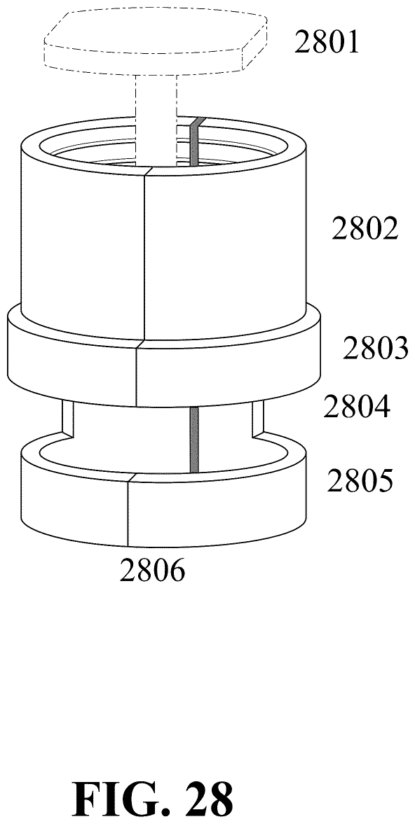

FIG. 28 shows a dosing guide 2802 with plunger 2801 shown in phantom. Also shown is a cut-out portion 2804 between 2803 and 2805. This cut-out portion 2804 allows the finger rests of a syringe barrel to extend out and through the dosing guide when the dosing guide is attached to the syringe and its finger rests. This arrangement is similar to the embodiment of the dosing guide shown in FIG. 27, and prevents relative movement between the dosing guide 2802 and a needle syringe contained therein. Also shown is 2806 which is a clam-shell opening. When 2806 is opened, a syringe barrel and finger rest can be engaged by the dosing guide and the dosing guide can be closed around the syringe barrel at position 2806. This arrangement is similar to the embodiment shown in FIG. 19, and allows for parts of the dosing guide to move relative to one another, which creates an opening to receive or remove the needle syringe from the dosing guide 2802.

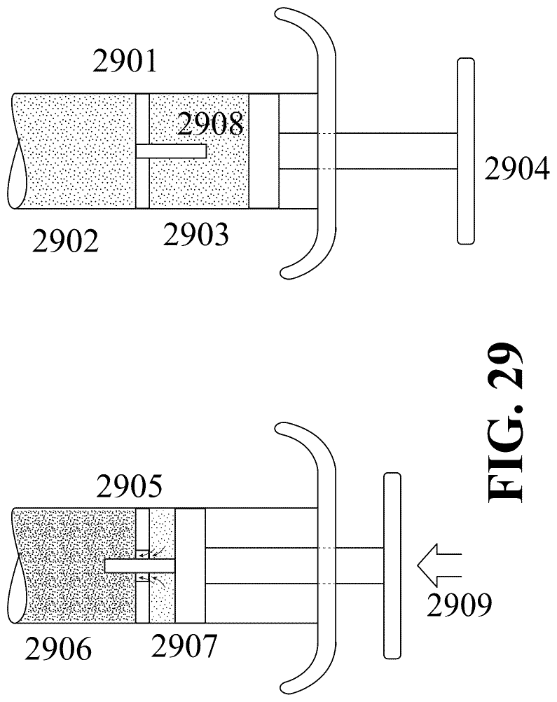

FIG. 29 shows a barrel 2901 having two chambers—2902 and 2903. One chamber—2902—can include a drug powder. One chamber—2903—can include a solution of a water-soluble solvent and a bioabsorbable polymer. Separating the two chambers is septum 2908. When plunger 2904 is depressed as in 2909, the septum 2908 is breached and the solution in chamber 2907 can mix with the drug powder in chamber 2906.

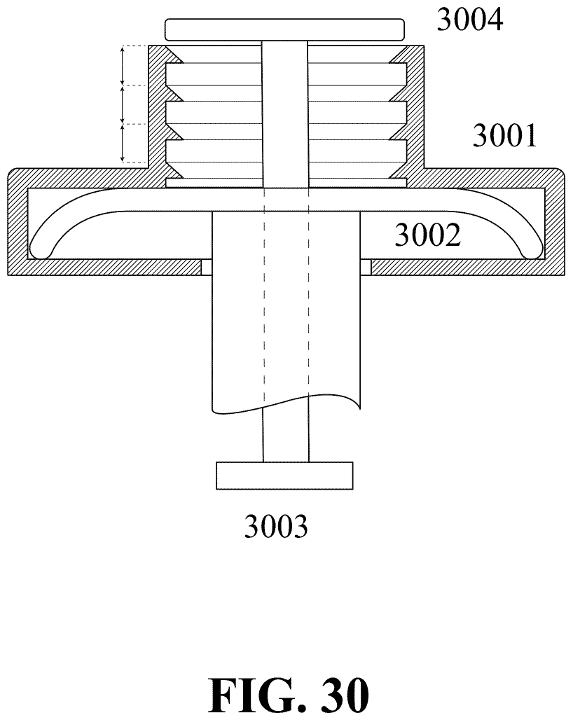

FIG. 30 is a variant of the dosing guide shown in FIG. 16. As shown, the dosing guide 3001 surrounds finger rests 3002. Also shown is plunger 3004 and 3003. In FIG. 30, the detents have a triangular configuration, compared to the semicircular configuration of the detents shown in FIG. 16. In alternative embodiments, the detents may have any desired shape. Regardless of the shape detents, spacing between adjacent detents may correspond to a single unit volume or incremental fractions of a unit volume, as discussed above. Additionally, as discussed above, regardless of the shape of the detents, the detents may be configured to resist movement of the plunger to facilitate administration of unit volumes and may be configured to provide haptic or audible feedback to a user to signal that the plunger has moved between detents.

While discrete embodiments have been shown and described above in regard to the dosing guide, it is understood that the disclosed features are not exclusive to each described embodiment. Instead, various features may be combined into a dosing guide as desired. For example, the shape of the detents of FIG. 30 may be used with the arrangement of the cut-out portion shown in FIG. 28.

In some cases, a SRF delivery dosing guide can be included with the needle syringe to assist with accurate dispensing of unit volumes. This dosing guide is attached to the proximal syringe plunger body 22 exposed outside of the needle. When attached the dosing guide will only enable syringe plunger translation to deliver the precise unit volume of e.g., 0.05 mL (50 micro-liters). The travel distance will be calibrated based on the inner diameter of the barrel. Or accompanying the dosing guide will be a lookup table or chart (provided with, e.g., an Instructions for Use) that indicates the volume dispensed using the guide for differently sized syringes. For example, a 1 mL syringe barrel has an inner diameter of 5 millimeters. In order to dispense a unit volume of 0.05 mL the dosing guide would have a distance LDC of 2.55 mm for each unit volume. The dosing guide can be inserted flush with the proximal body of the syringe barrel while attached to the plunger. After each unit volume injection, it can be removed and re-loaded for the next unit volume, or a total volume may be dispensed without the need to remove the dose guide, depending on the unit volume size and/or total volume needed.

The dose guide may be used in the following manner. First, the dead volume is removed from the needle. Next the dose guide is secured to the syringe by wrapping it around the barrel (hinge, such as a living hinge, FIG. 24) and securing the opposite ends by a clip. The plunger head should then rest against a detent or additional composition expelled until it rests against the topmost detent. When the plunger head is then advanced through one or more detents the unit volumes are dispensed through the needle tip, in registration with the number of detents over which the plunger head passed.

The syringes in FIGS. 2A and 2B are sized to enable a medical professional to accurately dispense unit volume amounts (e.g., 0.05 ml or 0.1 ml) at discrete/separate locations within a target tissue. The syringe may be pre-filled to hold a sufficient total volume for fully treating a patient (e.g., 4 unit volumes injectate at different locations in the prostate tissue). In some embodiments, an applicator portion may include syringe chambers for holding the SRF drug and drug carrier separate from each other, a mixing element for combining the drug and drug carrier such as a static mixing Y-adapter that feeds into a narrow and long, or high slenderness (L/D) ratio needle 28. Alternatively, the drug, drug carrier, and solvent may be mixed using two syringes connected with an adapter, with back and forth plunging to mix the drug, drug carrier, and polymer. Alternatively, the two syringes may connect directly to mix using mated female and male luer fittings at the distal location of each syringe. The applicator portion may have markings demonstrating measurement of lengths of needle insertion or composition volume.

The syringe control needed for dispensing unit volumes of a composition comprising a SRF from the barrel 22 through the needle tip 28 (FIGS. 2A-2B) or, in the case of a transurethral needle barrel (FIG. 2C) becomes challenging either when a long narrow needle is used, or individual injections do not exceed a unit volume amount. Both of these things are present according to the disclosure, as explained earlier. It was found that for the ranges of SRF formulations found suitable to achieve the goals of treatment and being deliverable in a composition in unit volume amounts, the following values for L_{b}, D_{b}, Lₙ, and Dₙ can apply. The needle length Lₙ may be from 35 cm to 70 cm, more preferably from 25 cm to 45 cm. The needle length Lₙ may be from 35 cm to 70 cm for transurethral. The needle length Lₐ may be 10 cm to 20 cm for transrectal. The lumen diameter Dₙ may be 0.03 cm, or between 0.026 and 0.0413 cm, and in some embodiments 0.02 cm up to 0.04 cm. The needle's slenderness ratio (Lₙ/Dₙ) may be between 600 and 2100, or between 600 and 1350.

In some embodiments, the syringe barrel's slenderness ratio (L_{b}/D_{b} may be 5 and 50, or between 10 and 30) is an important factor to consider. Barrel slenderness ratios in these ranges were found to help with more accurately dispensing low unit volumes of the composition.

In some embodiments, the syringe barrel's slenderness ratio (L_{b}/D_{b} may be 5 and 50, or between 10 and 30) is an important factor to consider. Barrel slenderness ratios in these ranges were found to help with more accurately dispensing low unit volumes of the composition.

A desired injection volume control and precision custom elongated glass body gastight delivery syringe can be utilized that provides increased accuracy of volume graduation markings on the syringe. Instead of volume graduations these delivery device markings can also show directly the needle calibrated dead volume and drug delivery drug dosage in mg for each injection for the user.

The pre-filled syringe preferably contains the composition as a pre-mixed solution for each injection, which results in delivery of a primarily amorphous drug already dissolved in the polymer carrier for faster drug release and enhanced onset of efficacy then later slower sustained release of primary crystalline drug.

In some embodiments, an SRF can be provided with polymer injectate and drug powder in the separate delivery syringes (mentioned above) and mixed by the user on or about the time of treatment and placed into the syringe barrel. This would allow for a crystalline or semi-crystalline drug directly into the injectate to increase drug retention time and exposure time and reduce systemic drug loss given that some drug would remain in a crystalline or semi-crystalline form after user preparation.

In some embodiments, an SRF generating composition can be mixed with an ultrasound microbubble contrast agent to further enhance visibility of injectate under transrectal ultrasound guidance to enable precise location control of injection within a prostate capsule within each lobe of a prostate to further minimize potential for drug loss to the urethra, surrounding organs and systemic circulation.

During the course of testing different formulations for an efficacious SRF, the inventors had sought to find a parameter or relationship that they could use to discount a formulation as unsuitable because it could not be delivered reliably. This was much needed because the iterative process of conceiving a certain formulation then evaluating whether it, in combination with the delivery vehicle could satisfy all three characteristics of prostate treatment was a long process. To find a formulation that eventually met those requirements, only to find out that it could not be reliably delivered in the small volumes needed using a long narrow needle, made the process much more labor intensive.

Moreover, it should be emphasized that the objective in matching a composition with a needle syringe was to not compromise more than needed, on the effectiveness of the SRF, in order to ensure its deliverability. The inventors sought an advantageous solution, not simply a way to save time or reduce the amount work needed to match an acceptable SRF with a delivery device. Unexpectedly, it was discovered that there is a numerical range to inform whether a unit volume v of a composition comprising an SRF (as represented by its absolute viscosity p) can be accurately and repeatedly delivered using a needle with length L and lumen diameter D.

Needle IDs smaller than 25 G (0.3 mm), or larger than 18 G (0.8 mm) are excluded from certain embodiments herein.

Needles smaller than 15 cm in needle length, or larger than 75 cm in needle length are excluded from certain embodiments herein.

When adopting this numerical range as a requirement for the syringe needle with composition, in addition to the required three characteristics for prostate treatment, a more effective SRF formulation is found, because the value addresses the deliverability problem and without unnecessarily compromising the SRF effectiveness. The numerical range is computed using the K-Injectate Rating or KIR, defined as follows:

$$KIR = \frac{μ {L}^{2}}{vD} ( 1 {0}^{- 6} )$$

For a KIR between 40 and 400, with units of centipoise (cP) per unit area, one may conclude that the SRF can be reliably and consistently delivered in unit volume increments using a needle having a length L, lumen diameter D, a composition's absolute viscosity p and a unit volume v. For transperineal or transrectal treatments, the KIR is defined for L from 10 to 30 cm, more preferably from 15 to 20 cm, a lumen diameter D of 0.06 cm, or from 0.06 to 0.08 cm, and in some embodiments 0.02 cm up to 0.12 cm. For transurethral treatments, the KIR is defined for L from 35 cm to 70 cm, and lumen diameter from 0.02 cm to 0.05 cm. The needle's slenderness ratio (L/D) may be between 200 and 400, or between 250 and 350. In some other embodiments, the needle's slenderness ratio (L/D) may be between 167 and 500.

In some embodiments, the KIR may be between 10 and 1000 for transurethral administration.

In some embodiments, the KIR may be between 16 and 1200 for transurethral administration.

In some embodiments, the KIR may be a number in Table 10 for transurethral administration.

In some embodiments, the KIR may be between 10 and 1000 for transrectal or transperineal administration.

In some embodiments, the KIR may be between 16 and 1200 for transrectal or transperineal administration.

In some embodiments, the KIR may be a number in FIG. 14A or 14B for transrectal or transperineal administration.

In some embodiments KIR may be between 10 and 1000. The higher ranges may be preferred in situations where a more delayed release rate and/or higher rate of gelation is desired, as well as smaller unit volumes.

TABLE 3 (shown in FIGS. 3A, 3B) discloses embodiments of an apparatus for delivery of a unit volume of a composition comprising an SRF according to the disclosure.

FIGS. 3A and 3B also disclose embodiments of an apparatus for delivery of a unit volume of a composition comprising an SRF according to the disclosure and intended for access to prostate by transrectal or transperineal method.

FIGS. 14A and 14B also disclose embodiments of an apparatus for delivery of a unit volume of a composition comprising an SRF according to the disclosure and intended for access to prostate by transurethral method.

As explained earlier, it is desirable to use a long, slender needle for injecting the relatively high-viscosity composition comprising a SRF that will produce the efficacious result without concomitant, adverse effects on the patient's health due to diffusion, infection, or pain/discomfort during or after the procedure. And it is also important that the medical professional treating the patient has adequate control over the volume of composition injected to enable the dispensing of a unit volume at each injection site, for volume control is necessary to achieve the desired clinical outcome. The ranges in TABLE 3 and FIGS. 14A and 14B for KIR are believed effective in identifying the composition satisfying the first two characteristics for prostate treatment according to the disclosure, and the syringe needle satisfying the third characteristic for prostate treatment according to the disclosure.

The lower end of the KIR range in TABLE 3 and FIGS. 14A and 14B represents compositions that while relatively easy to dispense in small unit volumes (for a fixed needle length, e.g., 15 cm), have a minimal amount of viscosity representative of an SRF's ability to satisfy two of the characteristics for prostate treatment (efficacy, sustained release, and minimal uncontrolled drug diffusion), shorter needle lengths and higher unit volumes. The upper end of the KIR range in TABLE 3 and FIGS. 14A and 14B represent (for a fixed needle length, e.g., 20 cm) compositions having higher viscosity, longer needle lengths, and smaller unit volumes. For these compositions, the two characteristics for prostate treatment will be easily satisfied when the formulation is injected, but the viscosity, if any higher, might challenge the health professional's ability to deliver a total volume of composition in unit volume amounts, especially along a longer needle length. As such, the capabilities of the delivery device are limiting for high KIR values.

In addition to KIR, in some embodiments the syringe barrel's slenderness (L_{b}/D_{b} may be 5 and 50, or between 10 and 30) can be an important factor to consider as well. Barrel slenderness ratios in these ranges were found to help with more accurately dispensing low unit volumes of the composition and may be preferred to satisfy the deliverability requirement (third characteristic).

In some embodiments, including any of the foregoing, the apparatus herein includes a needle having depth limiters and selectable unit-volume dispensing functions.

FIG. 10B is a schematic side-view illustration of a syringe having a depth limiter for not exceeding a predetermined depth of needle injection, and for dispensing a pre-determined unit volume of sustained release formulation (SRF) into a prostate. The physician guidance provided by the depth marker and selectable unit-volume dispensing feature enables for more ease with operating within the identified ranges of KIR.

Following description provides additional detailed information on embodiments of an SRF and the various constituents and properties therefore that may be included in a SRF for treating BPH using a needle syringe according to the disclosure.

Drug or drug combinations used in the SRF include a cytostatic drug, cytotoxic drug, and/or other drugs. The other drug(s) may be used by themselves (i.e., the “other drug(s)” are the only active agents in the SRF), or in combination with the cytostatic drug or cytotoxic drug as part of the medical procedure for treatment of BPH. For example, the other drug(s) may be administered before the composition injected into the target tissue using a needle syringe, or the other drug(s) may be included in the composition with the SRF. Or the other drug(s) may be administered after the SRF containing the cytostatic drug or cytotoxic drug is administered to the target tissue.

These other drugs, which may be administered with, or instead of the cytostatic or cytotoxic drug, include alpha blockers or 5-alpha reductase inhibitors. Alpha blockers may include and are not limited to terazosin, doxazosin, tamsulosin, alfuzosin, and silodosin. 5-alpha reductase inhibitors may include and are not limited to finasteride and dutasteride. Anti-inflammatory drugs may include but are not limited to corticosteroids such as dexamethasone, fluticasone propionate, triamcinolone acetonide, mometasone furoate, prednisone, hydrocortisone, estradiol, clobetasol, and budesonide. Non-steroidal drugs may include acetaminophen, ibuprofen, and naproxen. These other drug types may block cytokine activity or inhibit binding of cytokines to inhibit inflammatory signals such as anti-IL1, anti-IL 2, anti-IL3, anti-IL4, anti-IL8, anti-IL15, anti-IL 18, anti-MCP 1, anti-CCR2, anti-GM-CSF, anti-TNF antibodies and others. Combinations of the above drugs, and herein, may also be administered together.

Five alpha reductase inhibitors reduce the prostate volume by 50% when given orally. Minimal reduction occurs in less than six months. An up to 50% reduction in prostate volume is expected in 12-24 months or possibly longer with appropriate therapy.

Alpha blockers can also be used to treat symptomatically at the time of procedure by blocking the alpha receptor and relaxing the prostate smooth muscle. Alpha blockers, five alpha reductase inhibitors or both may be co-formulated with cytostatic or cytotoxic drugs in the SRF.

When expressing a % of a substance in the SRF, the % of that substance may be expressed in terms of a percent weight of the drug(s) to the overall weight of the SRF (“% X by weight”), or to the overall volume of the SRF (“% X by vol”). Unless stated otherwise the percent dosage % will, by default, refer to a % by weight to the total measured SRF. Unless stated otherwise, weights are given in grams (“g”) or milligrams (“mg”), molecular weight in kilo-Daltons (“kDa”), volume in microliters (“μL”) or milliliters (“mL”), and viscosity units are expressed as inherent viscosity (i.e., the ratio of the natural logarithm of the relative viscosity to the mass concentration of the substance, such as a polymer. The unit of inherent viscosity is deciliters per gram (dL/g). A different measure of viscosity is intrinsic viscosity, which is a measure of a solute's contribution to the total viscosity. Another viscosity is dynamic viscosity or absolute viscosity, the units of which are centimeter-gram-seconds, also known as centipoise (cP).

The SRF may comprise 0.1-60% of a polymer composition, or more preferable 30-50% of a polymer composition. The SRF may comprise 0-80% solvent; however, the solvent is the majority component and is present as the remainder after considering the amount of drug and bioabsorbable polymer. The drug to polymer weight ratio of the SRF may be 1:100, 1:50, 1:25, 1:20, 1:10, 1:5, 1:2, 1:1, 2:1, or 5:1. The SRF, once located at the target tissue, may release 1-10%, or 11-50% of the drug load in less than 24 hours (h), 24-72 hours, 3-7 days, 1-4 weeks, 1-3 months or more than 3 months. The SRF may release 80-100% in 24-72 hours, 3-7 days, 1-4 weeks, 1-3 months or more than 3 months.

The drug carrier may be a polymer composition including silk-elastin like protein polymers, Pluronics F68 or F127 or a combination thereof, poly(ε-caprolactone) (PC), polylactides (PLA), poly(D,L-lactide) (PDLA), poly(ortho esters), polyanhydrides, polycarbonates, polyethylene glycol (PEG), polyethylene oxide (PEO), polyesteramides, and any combinations thereof including block and random co-polymers such as but not limited to poly(lactide-co-glycolide) (PLGA) and PLGA-PEG-PLGA. More specifically the PLGA composition may consist of poly(D,L-lactide-co-glycolide) (50:50), poly(D-lactide-co-glycolide) (50:50), poly(L-lactide-co-glycolide) (50:50), poly(D,L-lactide-co-glycolide) (65:35), poly(D-lactide-co-glycolide) (65:35), poly(L-lactide-co-glycolide) (65:35), poly(D,L-lactide-co-glycolide) (75:25), poly(D-lactide-co-glycolide) (75:25), poly(L-lactide-co-glycolide) (75:25), poly(D,L-lactide-co-glycolide) (85:15) or a mixture thereof. The PLGA may be end-capped with ester, acid, alcohol, thiol or other end-groups. The inherent viscosity of the PLGA polymer may vary from 0.1 dL/g to greater than 1.0 dL/g. The molecular weight of the PLGA polymer may vary from 10 kDa up to 150 kDa. The polymer may be linear, branched, hyperbranched, dendritic, have a star structure, or be a dendrimer-like star polymer.

Additional embodiments of the drug carrier, SRF and composition follow.

The drug carrier may include a polymer composition including poly(lactide-co-glycolide), poly(D,L-lactide-co-glycolide), poly(D,L-lactide), ester end capped poly(D,L-lactide-co-glycolide) (50-50), ester end capped poly(D,L-lactide-co-glycolide) (65-35), ester end capped poly(D,L-lactide-co-glycolide (75-25), ester end capped poly(D,L-lactide-co-glycolide (85-15), acid end capped poly(D,L-lactide-co-glycolide) (50-50), acid end capped poly(D,L-lactide-co-glycolide) (65-35), acid end capped poly(D,L-lactide-co-glycolide (75-25), acid end capped poly(D,L-lactide-co-glycolide (85-15), ester end capped poly(D-lactide-co-glycolide) (50-50), ester end capped poly(D-lactide-co-glycolide) (65-35), ester end capped poly(D-lactide-co-glycolide (75-25), acid end capped poly(D-lactide-co-glycolide) (50-50), acid end capped poly(D-lactide-co-glycolide) (65-35), acid end capped poly(D-lactide-co-glycolide (75-25), ester end capped poly(L-lactide-co-glycolide) (50-50), ester end capped poly(L-lactide-co-glycolide) (65-35), ester end capped poly(L-lactide-co-glycolide (75-25), acid end capped poly(L-lactide-co-glycolide) (50-50), acid end capped poly(L-lactide-co-glycolide) (65-35), acid end capped poly(L-lactide-co-glycolide (75-25), ester end capped poly(D,L-lactide-co-glycolide), acid end capped poly(D,L-lactide-co-glycolide), or combinations thereof.

In some embodiments the drug carrier may include a bioabsorbable polymer composition and the inherent viscosity of the polymer composition is between 0.1-1.0 dL/g, 0.1-0.6 dL/g, or 0.1 to 0.4 dL/g or 0.1 to 0.3 dL/g and the ratio of DL-lactide to glycolide is from 30/70 up to 90/10, 95/5, or 85/15.

In some embodiments the composition may include a bioabsorbable polymer at a concentration of 10-55%, 15-50% by wt. of the bioabsorbable polymer composition, 85-45% by wt. of the solvent and 0.5%-30% by wt. drug; 1%-20% by wt. of drug, or 1%-10% by wt. of drug.

The drug carrier (polymer composition) may generally be in the form of amorphous or semi-crystalline, homogenous, or phase-separated, and provided in the form of a liquid solution or, suspension, gel, or as a solid implant processed by injection molding, casting or extrusion. The biodegradable polymer composition is preferably chosen to substantially biodegrade in a period of about 1 to 3 months, 3 to 6 months or 6 to 12 months.

In some embodiments, including any of the foregoing, the SRF is a solution comprising a drug, solvent, and bioabsorbable polymer.

In some embodiments it may be desirable to formulate the SRF so that the drug carrier is fully biodegraded before the next treatment, e.g., 3 months, 6 months or 12 months after the prior treatment. For example, the polymer would have a ratio of glycolide to lactide of 70:30 up to 15:85 for a less hydrophobic structure (faster degradation) and/or an inherent viscosity less than about 1.0 dL/g or more preferably less than 0.3 dL/g.

A polymer composition, when forming a constituent of the SRF, is a polymer composition that enables or achieves a desired “sustained release” of the one or more drugs to the target tissue. In some embodiments, the polymer composition enables or achieves at least 50%, or up to about 100%, or substantially all drug release between 30 and 90 days, through a combination of diffusion and degradation. In other embodiments up 100% of drug release occurs from 90 to 120 days from treatment. Preferably, there is an initial burst (e.g., up to 50% of drug) followed by a substantially reduced rate of release over the next following month, or several following months following treatment. For example, the drug has a release rate of between 5% to 50% during the first 24 hours from injecting the composition into the prostate and the drug has a release rate of no more than 10% to 75% over the first month, 25% to 95% over the first three months, and/or 50% to 100% over the first six months.

As discussed earlier, the SRF must keep drug exposure to the prostate and minimize leak or flow to other surrounding organs. In addition, the SRF should not take up too much volume in the prostate. A desired shape of the drug release curve could be a burst of drug and early tissue exposure for fast efficacy and then reduced rate of drug release over a 3-6 month period for sustained efficacy. In some examples the drug release curve could be a burst of drug and early tissue exposure for fast efficacy and then reduced rate of drug release over a 1-4 month period for sustained efficacy. For example, a fast burst release rate followed by a reduced rate was achieved with N-methyl pyrrolidone (NMP) solvent that is water soluble. This formulation can provide a high release within the first 24 hours or 48 hours, or within the first 1 week, two weeks, or up to 3 months, followed by a reduced rate of release.

FIG. 3C shows a Drug Release Curve for Sustained Release Formulation (SRFs) A, B and C. These exemplary SRFs provide a burst followed by gradual release of drug over a period of up to 6 months. SRF A releases about 25% of the drug within the first month, followed by a slow, gradual release where about 95% is released at six months. SRF B has an initial burst of about 50% within the first month, followed by about 100% release at two months. SRF C has an initial burst of about 75% within the first month, with about 100% release at about two months.

TABLE 4A below shows examples of SRF formulations for each of SRF A, B and C. Examples A1, A2 exhibit approximately the same release rate characteristics as SRF A in FIG. 3C. Examples B1, B2 exhibit approximately the same release rate characteristics as SRF B in FIG. 3C. Examples C1, C2 exhibit approximately the same release rate characteristics as SRF C in FIG. 3C. TABLE 4B shows examples of drug released over time.

TABLE 4A
Species of SRF formulations for SRF A, B and C in FIG. 3C

| SRF | polymer | drug | % wt. drug v. polymer | Solvent |
|---|---|---|---|---|
| A1 | PLGA8515 | Sirolimus | 1-2% drug, 50% polymer | NMP |
| A2 | PLGA8515 | Paclitaxel | 1-2% drug, 50% polymer | NMP |
| B1 | PLGA7525 | Sirolimus | 3-5% drug, 45-47% | NMP |
| | | | polymer | |
| B2 | PLGA7525 | Paclitaxel | 3-5% drug, 45-47% | NMP |
| | | | polymer | |
| C1 | PLGA5050 | Sirolimus | 1-5% drug, 45-55% | NMP |
| | | | polymer | |
| C2 | PLGA5050 | Paclitaxel | 1-5% drug, 45-55% | NMP |
| | | | polymer | |

TABLE 4B
plotted ranges for SRF A, B & C (FIG. 3C)

| Time | SRF A | SRF B | SRF C |
|---|---|---|---|
| (months) | (drug release %) | (drug release %) | (drug release %) |
| 0 | 0 | 0 | 0 |
| 0.5 | 30 ± 20 | 50 ± 25 | 75 ± 25 |
| 1 | 50 ± 20 | 75 ± 15 | 95 ± 5 |
| 2 | 60 ± 15 | 95 ± 5 | 100 |
| 3 | 70 ± 10 | 100 | |
| 6 | 95 ± 5 | | |

The SRF is delivered to the target tissue in the form of a composition in liquid form (i.e., the SRF's drug(s) and drug carrier are in solution, or in suspension in a solvent when in the delivery vehicle) that gels upon contact with water at the target tissue. The SRF may be made by dissolving the SRF components in a suitable solvent. Suitable solvents for these embodiments include water, N-methylpyrrolidone (NMP), dimethyl sulfoxide (DMSO), 2-pyrrolidone, propylene carbonate, caprolactam, triacetin, alcohols, benzyl benzoate, ethyl acetate, triethyl citrate, benzyl alcohol, glyme (dimethoxyethane), diglyme, and other glycol ethers, and dichloromethane, or any mixture thereof. In some embodiments, including any of the foregoing, the solvent is N-methylpyrrolidone (NMP). In some embodiments, including any of the foregoing, the solvent is dimethyl sulfoxide (DMSO). In some embodiments, including any of the foregoing, the solvent is 2-pyrrolidone. In some embodiments, including any of the foregoing, the solvent is propylene carbonate. In some embodiments, including any of the foregoing, the solvent is caprolactam. In some embodiments, including any of the foregoing, the solvent is triacetin. In some embodiments, including any of the foregoing, the solvent is an alcohol or mixture of alcohols. In some embodiments, including any of the foregoing, the solvent is benzyl benzoate. In some embodiments, including any of the foregoing, the solvent is ethyl acetate. In some embodiments, including any of the foregoing, the solvent is triethyl citrate. In some embodiments, including any of the foregoing, the solvent is benzyl alcohol. In some embodiments, including any of the foregoing, the solvent is glyme (dimethoxyethane). In some embodiments, including any of the foregoing, the solvent is diglyme. In some embodiments, including any of the foregoing, the solvent is a glycol ether. In some embodiments, including any of the foregoing, the solvent is dichloromethane.

As explained earlier, the composition should be formulated at a concentration and viscosity that permits passage through a long, narrow needle having a slenderness ratio within the prescribed ranges deliverable in low unit volumes, and controls the drug release, and drug distribution. Drug to polymer ratios, which influence the viscosity of the composition, may vary from 0.025 to 2.0. Drug and polymer concentrations in solvent may range from 0.1 wt % up to 60 wt % or more preferably 30 wt % to 50 wt %. Unit volumes may range from 0.025 mL up to 0.15 mL per injection or more preferably from 0.05 mL to 0.10 mL per injection. Total volume of the composition for a treatment may range from 0.050 mL up to 1.5 mL or more preferably 0.10 mL to 1.0 mL. Overall dosage of drug provided in the SRF can range from 50 ug up to 200 mg or more preferably from 5 mg to 50 mg. For example, a composition containing the SRF is 300 μg of sirolimus or paclitaxel dissolved with 3000 μg (“μg”—micrograms) poly (D, L-lactide-co-glycolide) (85:15) in NMP at a 50 wt % concentration.

The following disclosure provides further, non-limiting examples for treating prostatic hyperplasia tissues within a patient using an apparatus or medical device according to the disclosure and provides a description of, and results from the second pre-clinical study referred to earlier.

As discussed above, the local drug delivery device is a syringe needle for delivery of the composition including the SRF to the target tissue, as earlier shown and described with reference to FIGS. 2A and 2B. In other embodiments, the applicator portion of a delivery vehicle may include syringe chambers for holding the drug and drug carrier separate from each other, a mixing element for combining the drug and drug carrier such as a static mixing Y-adapter that feeds into a narrow and long, or high slenderness ratio needle. Alternatively, the drug and drug carrier may be mixed using two syringes connected with an adapter, with back and forth plunging to mix the drug and drug carrier. The syringe needle may be imaged under ultrasound to visualize the therapy. Echogenicity may be enhanced by dimpling of the applicator needle or coatings. Echogenicity may also be enhanced by incorporation of contrast enhancing agents such as microbubbles, sulfur hexafluoride, octofluoropropane, air, lipid and/or albumin shells. The applicator may have components for attachment to a rectal ultrasound probe. The applicator may have markings demonstrating measurement of lengths of needle insertion or composition volume. The applicator may have a user-friendly handle and plunger to comfortably deliver the needle to the target tissue. An applicator needle size may range from 16 G up to 25 G. The needle length may range from 10 cm to 30 cm (e.g., 15 cm, 20 cm length) in order to reach the target tissue from a transrectal or transperineal approach.

### 2021 Canine Study

### Example

A preclinical canine study was conducted to evaluate the safety and feasibility of a composition comprising an SRF (SRF1) injected into the prostates of six canines, each having enlarged prostates post weekly treatments by testosterone injection over a period of 12 weeks. The SRF1 treatment was delivered 12 weeks post the initial testosterone injections. The composition comprising SRF1 was delivered to these BPH canine models in the following manner:

SRF1 with PLGA 5050 and NMP included 5 mg paclitaxel delivered to a BPH canine model prostate (N=4) in a 100 microliter composition by injecting two 50 microliter injections of the composition into each side or lobe of the prostate while imaging by transrectal ultrasound guidance using a 20 G×20 cm Chiba needle delivered via a 250 microliter gastight glass delivery syringe. The composition was easy for the user to deliver and visualize under ultrasound without discomfort to the animal. A total of 2 injections (50 microliter each) were made to each BPH canine model to deliver SRF1.

10 mg paclitaxel was delivered to the BPH canine model prostate (N=2) in a 200 microliter (μl) composition by injecting two 100 μL of the composition into each side or lobe of the prostate while imaging by transrectal ultrasound guidance using a 20 G×20 cm Chiba needle delivered via 500 microliter gastight glass delivery syringe. The composition was readily observable by the user to deliver and visualize under ultrasound and did not cause any discomfort to the animal. A total of 2 injections (100 microliter each) were made to each BPH canine model to deliver SRF1.

Blood samples were collected from all animals at 0, 1, 3, 7, 14, 30, 60 and 90 days post treatment or until the animal was euthanized. 30 and 90 days following the procedure the models were humanely euthanized and evaluated to determine whether there were any acute, toxic effects of the injectate. At the time the animals were euthanized, tissue samples were also collected from the prostate, bladder, and urethra to determine the residual drug concentrations. The day-to-day behavior of all animals was also studied over the 30-and-90 days periods following the procedure.

Data collected from each of the models (i.e., the 30- and 90-day studies):
Prostate Morphology and Prostate Size/Weight Baseline and Post-Treatment (Ultrasound)
Gross Necropsy and Camera/Microscope Imaging at Termination, Fixation
Histology Imaging at Treatment Site at Termination (H&E)

TABLE 5 shows prostate volume (cc, or cm³) as measured by ultrasound at baseline (i.e., before the animal received SRF1) and 4 weeks and 12 weeks post SRF1 treatment. TABLE 5 shows a reduction in prostate volume (%) post SRF1 treatment relative to baseline.

TABLE 5

| | | Prostate Volume by | | | Reduction from Baseline in | |
|---|---|---|---|---|---|---|
| | | Ultrasound (cm³ or cc) | | | Prostate Volume (%) | |
| | Drug Dose | | 4 weeks | 12 weeks | 4 weeks | 12 weeks |
| Animal# | (mg) | Baseline | post SRF1 | post SRF1 | post SRF1 | post SRF1 |
| 50 | 5 | 39.2 | 10.6 | — | −72.9 | — |
| 45 | 5 | 26.4 | 7.54 | — | −71.4 | — |
| 42 | 10 | 29.7 | 5.87 | — | −80.3 | — |
| 46 | 5 | 19.5 | — | 13.7 | — | −29.9 |
| 52 | 5 | 22.2 | — | 8.65 | — | −61.0 |
| 44 | 10 | 44.1 | — | 8.77 | — | −80.1 |

The BPH canine models in the study are distinguished by number. Animal numbers 45, 46, 50, and 52 received the 5 mg drug dose of SRF1. BPH canine animal numbers 42 and 44 received the 10 mg drug dose. Prostate volumes decreased by as much as 80% 4 weeks and twelve weeks from treatment (animal 44). For example, the prostate volume for animal 50 reduced by 72.9% ([(10.6/39.2)−1]*100=−72.9%).

TABLE 6

| | Prostate Volume by | | % Prostate Volume | |
|---|---|---|---|---|
| | Ultrasound (cc, Mean) | | Reduction (Mean) | |
| Dose | 4 weeks | 12 weeks | 4 weeks | 12 weeks |
| (mg) | post SRF1 | post SRF1 | post SRF1 | post SRF1 |

TABLE 7

| | Overall (n = 3) | | 5 mg dose (n = 2) | | 10 mg dose (n = 1) | |
|---|---|---|---|---|---|---|
| | 30-day (μg/g) | | 30-day (μg/g) | | 30-day (μg/g) | |
| | | Standard | | Standard | | Standard |
| Tissue | Mean | Deviation | Mean | Deviation | Mean | Deviation |
| Main | 0.16 | 0.14 | 0.10 | 0.12 | 0.29 | NA |
| Bladder | | | | | | |
| Bladder neck | 0.05 | 0.06 | 0.02 | 0.02 | 0.12 | NA |
| Adventitia / | 1.20 | 2.05 | 1.80 | 2.51 | 0.02 | NA |
| Adipose | | | | | | |
| Tissue | | | | | | |
| Prostate tx1 | 16.01 | 6.91 | 20.00 | 0.00 | 8.03 | NA |
| Prostate tx2 | 17.07 | 5.08 | 20.00 | 0.00 | 11.20 | NA |
| Prostate adj1 | 13.92 | 9.59 | 19.45 | 0.78 | 2.87 | NA |
| Prostate adj2 | 11.15 | 7.37 | 9.17 | 9.23 | 15.10 | NA |
| Prostate | 6.23 | 9.17 | 9.12 | 10.87 | 0.46 | NA |
| distal 1 | | | | | | |
| Prostate | 1.46 | 1.75 | 2.17 | 1.76 | 0.04 | NA |
| distal 2 | | | | | | |
| Prostate | 9.28 | 10.03 | 13.86 | 8.68 | 0.12 | NA |
| remains | | | | | | |
| Urethra | 1.10 | 1.82 | 1.60 | 2.26 | 0.10 | NA |
| proximal | | | | | | |
| Urethra | 0.06 | 0.05 | 0.03 | 0.03 | 0.11 | NA |
| distal | | | | | | |

| | | | | |
|---|---|---|---|---|
| 5 | 9.09 | 11.2 | −72.1 | −45.5 |
| 10 | 5.87 | 8.77 | −80.3 | −80.1 |

TABLE 6 shows the mean average change in canine prostate volume (cc) as a function of the drug dose injected (5 mg and 10 mg) and measured by transrectal ultrasound, post SRF1 injections (4 and 12 weeks) and post SRF1 treatments (4 and 12 weeks).

TABLE 7 shows the overall (N=3) mean and standard deviation of drug concentrations (ag/g) in the various organ and tissue 30 days post SRF1 treatment. TABLE 7 also shows the mean (and standard deviation for 5 mg) of drug concentrations (jag/g) in the various organ and tissue 30 days post SRF1 treatment for the two doses delivered, 5 mg (N=2) and 10 mg (N=1).

TABLE 8

| | Overall (n = 3) | | 5 mg dose (n = 2) | | 10 mg dose (n = 1) | |
|---|---|---|---|---|---|---|
| | 90-day (μg/g) | | 90-day (μg/g) | | 90-day (μg/g) | |
| | | Standard | | Standard | | Standard |
| Tissue | Mean | Deviation | Mean | Deviation | Mean | Deviation |
| Main Bladder | 0.03 | 0.01 | 0.03 | 0.01 | 0.03 | NA |
| Bladder neck | 0.09 | 0.08 | 0.11 | 0.11 | 0.06 | NA |
| Adventitia / Adipose | 0.51 | 0.24 | 0.43 | 0.27 | 0.69 | NA |
| Tissue | | | | | | |
| Prostate tx1 | 103.97 | 179.29 | 155.60 | 219.77 | 0.71 | NA |
| Prostate tx2 | 10.52 | 15.20 | 14.22 | 19.49 | 3.12 | NA |
| Prostate adj1 | 3.71 | 5.32 | 5.46 | 6.19 | 0.22 | NA |
| Prostate adj2 | 4.64 | 6.76 | 6.94 | 7.73 | 0.04 | NA |
| Prostate distal 1 | 4.37 | 3.82 | 2.56 | 3.11 | 7.98 | NA |
| Prostate distal 2 | 1.83 | 2.72 | 2.69 | 3.21 | 0.10 | NA |
| Prostate remains | NA | NA | NA | NA | NA | NA |
| Urethra proximal | 0.51 | 0.53 | 0.22 | 0.22 | 1.10 | NA |
| Urethra distal | 0.29 | 0.35 | 0.39 | 0.43 | 0.08 | NA |

TABLE 8 shows the overall (N=3) mean and standard deviation of drug concentrations (μg/g) in the various organ and tissue 90 days post SRF1 treatment. TABLE 8 also shows the mean (and standard deviation for 5 mg) of drug concentrations (μg/g) in the various organ and tissue 30 days post SRF1 treatment for the two doses delivered, mg (N=2) and 10 mg (N=1).

TABLE 9

| | | | Eosinophilic | | | | | | | | | |
|---|---|---|---|---|---|---|---|---|---|---|---|---|
| | | | Amorphous | | | | | | | | | |
| | Acinar | | material | | Necrosis (0- | | | | Inflammation | | Fibrosis ( [symbol] | |
| Timepoint | dilation (0-4) | | (0-4) | | 4) | | Fibrin (0-4) | | (0-4) | | 4) | |
| (days) | Mean | StDev | Mean | StDev | Mean | StDev | Mean | StDev | Mean | StDev | Mean | St [symbol] |

Overall

| | | | | | | | | | | | | |
|---|---|---|---|---|---|---|---|---|---|---|---|---|
| 30 (n = 3) | 0.68 | 0.79 | 1.53 | 1.40 | 1.27 | 1.21 | 0.27 | 0.12 | 2.20 | 1.25 | 1.07 | 0. [symbol] |
| 90 (n = 3) | 1.00 | 0.00 | 0.80 | 0.80 | 0.47 | 0.42 | 0.00 | 0.00 | 1.20 | 0.87 | 0.33 | 0. [symbol] |

5 mg Drug Dose

| | | | | | | | | | | | | |
|---|---|---|---|---|---|---|---|---|---|---|---|---|
| 30 (n = 2) | 0.90 | 0.99 | 1.60 | 1.98 | 1.20 | 1.70 | 0.20 | 0.00 | 2.70 | 1.27 | 0.80 | 0. [symbol] |
| 90 (n = 2) | 1.00 | 0.00 | 1.20 | 0.57 | 0.70 | 0.14 | 0.00 | 0.00 | 1.50 | 0.99 | 0.00 | 0. [symbol] |

10 mg Drug Dose

| | | | | | | | | | | | | |
|---|---|---|---|---|---|---|---|---|---|---|---|---|
| 30 (n = 1) | 0.25 | NA | 1.40 | NA | 1.40 | NA | 0.40 | NA | 1.20 | NA | 1.60 | N [symbol] |
| 90 (n = 1) | 1.00 | NA | 0.00 | NA | 0.00 | NA | 0.00 | NA | 0.60 | NA | 1.00 | N [symbol] |

[symbol]

indicates data missing or illegible when filed

TABLE 9 summarizes histopathology scoring from histopathology H&E microscopic images post SRF1 injection in each of the canine BPH prostates. Scoring ranges from 0 to 4 (0=none, 1=minimal, 2=mild, 3=moderate and 4=marked/severe/complete). Minimal to moderate values of necrosis and inflammation over time are evident of the localized drug effect of necrosis observed in the histopathology images. Minimal to mild fibrosis that resolves over time from 30 days to 90 days is likely evident of the drug effect in reducing the prostate volume (StDev=Standard Deviation).

It is known that for aging men with a history of BPH and a reduced serum testosterone concentration the size of the prostate is not reduced when serum total testosterone concentration is reduced. See Xia, B.-W. et al, Relationship between serum total testosterone and prostate volume in aging men, Scientific Reports, 11, 14122 (2021).

As mentioned earlier, while prior methods may show efficacy in reducing BPH, they either may require a more invasive procedure (vs. localized treatment using the delivery device as disclosed herein, such as the needle used in the animal study), more frequent treatment due to diffusion or more generalized treatment of BPH raising the possibility of adverse effects because a higher dosage is needed to treat the area while accounting for leakage or diffusion of the drug to other areas. Adverse effects may include diminished urinary or sexual function. It is desired to have an effective treatment targeting only the target tissue and nowhere else (e.g., avoiding the urethra) and to perform the procedure in a less invasive manner for patient acceptance.

As mentioned earlier, both injection volumes of SRF1 showed significant reduction in prostate size, as reported in TABLE 5 and TABLE 6. Additionally, as shown in FIG. 7 and FIG. 8, there was very low systemic drug concentrations present in the blood, indicating that the agent was contained at the target tissue. The Example (pre-clinical study 2) indicates that an effective SRF injectate according to the disclosure (1) reduces prostate volume significantly over a 30 and 90 day period, (2) limits substantially all of the active agent to the target tissue, and (3) is capable of being delivered as a series of low unit volumes using a needle syringe. As for point (2), it should be mentioned that other known products for treating BPH by contrast have their active agents disperse significantly (systemic drug concentrations much higher than what is shown in FIGS. 7-8). Moreover, the study indicated a significant ratio of prostate 30-day drug concentration to maximum plasma drug concentration of at least about 10,000. As mentioned throughout, it is important to control diffusion of the agent as this can cause adverse effects on nearby tissue and organs. Additionally, in contrast to most prior or existing techniques for treating BPH, a comparatively small volume of SRF injectate is needed for efficacy. In the example (pre-clinical study) a unit volume of the composition comprising SRF1 was between 10-200 microliter per injection over 1-10 injections across each side of the prostate, or 50-100 microliter per injection over 1-5 injections across each side of the prostate. Prior approaches for treating BPH seek to maximize injection volumes for increased drug dosage. An SRF however does not require as high a drug dosage, because the drug is maintained in the target tissue.

Following are additional listing of disclosed embodiments with KIR values useful for treating BPH:

0.5 mL N-methyl pyrrolidone (NMP) was added to a vial with 0.25 g sirolimus and vortexed until dissolved. 50 microliter of drug solution was added to 2.5 mL of a 50 percent by weight PLGA8515A (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% by weight PLGA8515A (0.5 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% by weight PLGA8515E (0.3 dl/g) in NMP solution using syringe to syringe mixing with a female to female luer connector. 250 microliters in a 1 mL syringe were loaded into a 20 G×20 cm Chiba biopsy needle with depth markers. The “E” in PLGA8515E means ester-capped.

0.5 mL N-methyl pyrrolidone (NMP) was added to a vial with 0.25 g sirolimus and vortexed until dissolved. 50 microliter of drug solution was added to 2.5 mL of a 50% by weight PLGA8515E (0.5 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% by weight PLGA8515A (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% by weight PLGA8515E (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA8515A (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA8515E (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA8515A (0.5 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA8515E (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA8515E (0.3 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA8515A (0.5 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA8515E (0.7 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 200 microliter of drug solution was added to 2.5 mL of a 50% by weight PLGA8515 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 200 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g sirolimus and vortexed until dissolved. 50 microliter of drug solution was added to 2.5 mL of a 50% by weight PLGA5050 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% by weight PLGA5050 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g sirolimus and vortexed until dissolved. 200 microliter of drug solution was added to 2.5 mL of a 50% by weight PLGA5050 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515A (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515E (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515A (0.5 dl/g) in NMP solution using syringe to syringe mixing with a female to female luer connector. 250 microliters in a 1 mL syringe were loaded into a 20 G×20 cm Chiba biopsy needle with depth markers. 0.5 mL N-methyl pyrrolidone (NMP) was added to a vial with 0.25 g everolimus and vortexed until dissolved. 50 microliter of drug solution was added to 2.5 mL of a 50% by weight PLGA8515E (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515A (0.5 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515E (0.7 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515E (0.5 dl/g) in NMP solution using syringe to syringe mixing with a female to female luer connector. 800 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515A (0.7 dl/g) in NMP solution using syringe to syringe mixing with a female to female luer connector. 800 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515A (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA8515E (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 100 microliter of drug solution was added to 2.5 mL of a 50% by weight PLGA8515 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% by weight PLGA8515 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 100 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% by weight PLGA7525A (0.3 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA7525E (0.3 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA7525A (0.5 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA7525E (0.5 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA7525A (0.7 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA7525E (0.7 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA6535E (0.3 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA6535A (0.3 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA6535E (0.5 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA6535A (0.5 dl/g) in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA6535E (0.5 dl/g) in NMP solution using syringe to syringe mixing with a female to female luer connector. 500 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 200 microliter of drug solution was added to 2.5 mL of a 50% by weight PLGA6535A (0.5 dl/g) in NMP solution using syringe to syringe mixing with a female to female luer connector. 500 microliters in a 1 mL syringe were loaded into a 20 G×20 cm Chiba biopsy needle with depth markers

0.5 mL N-methyl pyrrolidone (NMP) was added to a vial with 0.25 g everolimus and vortexed until dissolved. 50 microliter of drug solution was added to 2.5 mL of a 50% by weight PLGA5050 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 100 microliter of drug solution was added to 2.5 mL of a 50% by weight PLGA5050 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% by weight PLGA5050 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 50 microliter of drug solution was added to 2.5 mL of a 50% by weight PLGA8515 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515A (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515E (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515A (0.5 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515E (0.5 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515A (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515E (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515A (0.5 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515E (0.5 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA8515A (0.7 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 500 microliter of drug solution was added to 2.5 mL of a 50% by weight PLGA8515E (0.7 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 200 microliter of drug solution was added to 2.5 mL of a 50% by weight PLGA8515 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 50 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 200 microliter of drug solution was added to 2.5 mL of a 50% by weight PLGA7525 in NMP solution using syringe to syringe mixing with a female to female luer connector. 250 microliters in a 1 mL syringe were loaded into a 20 G×20 cm Chiba biopsy needle with depth markers

0.5 mL N-methyl pyrrolidone (NMP) was added to a vial with 0.25 g paclitaxel and vortexed until dissolved. 100 microliter of drug solution was added to 2.5 mL of a 50% by weight PLGA6535A (0.3 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% by weight PLGA6535A (0.5 dl/g) in NMP solution using syringe to syringe mixing with a female to female luer connector. 500 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 500 microliter of drug solution was added to 2.5 mL of a 50% by weight PLGA6535E (0.3 dl/g) in NMP solution using syringe to syringe mixing with a female to female luer connector. 250 microliters in a 1 mL syringe were loaded into a 20 G×20 cm Chiba biopsy needle with depth markers.

0.5 mL N-methyl pyrrolidone (NMP) was added to a vial with 0.25 g paclitaxel and vortexed until dissolved. 500 microliter of drug solution was added to 2.5 mL of a 50% by weight PLGA6535E (0.5 dl/g) in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA5050A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA5050A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA5050A in NMP solution using syringe to syringe mixing with a female to female luer connector (#SRF1). 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA5050A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA5050E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA5050E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA5050E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA5050E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA8515A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA8515A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA8515A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA8515A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA8515E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA8515E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA8515E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA8515E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA6535A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA6535A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA5050A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA6535A in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA6535E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA6535E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA6535E in NMP solution using syringe to syringe mixing with a female to female luer connector. 250 microliters in a 1 mL syringe were loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight by weight PLGA6535E in NMP solution using syringe to syringe mixing with a female to female luer connector. 250 microliters in a 1 mL syringe were loaded into a 20 G×20 cm Chiba biopsy needle with depth markers.

See also the Table in FIGS. 14A and 14B, which is reproduced below:

TABLE 10

| Unit | | Needle | Needle | |
|---|---|---|---|---|
| Volume | Viscosity | Length | ID | KIR |
| (ml) | (cP) | (cm) | (cm) | (cP/cm²) |
| 0.5 | 267 | 35 | 0.0413 | 16 |
| 0.5 | 300 | 35 | 0.0413 | 18 |
| 0.5 | 300 | 35 | 0.0337 | 22 |
| 0.5 | 500 | 35 | 0.0413 | 30 |
| 0.5 | 533 | 35 | 0.0413 | 32 |
| 0.25 | 267 | 35 | 0.0413 | 32 |
| 0.25 | 300 | 35 | 0.0413 | 36 |
| 0.5 | 500 | 35 | 0.0337 | 36 |
| 0.25 | 300 | 35 | 0.03 | 49 |
| 0.25 | 500 | 35 | 0.0413 | 59 |
| 0.25 | 533 | 35 | 0.0413 | 63 |
| 0.25 | 500 | 35 | 0.0337 | 73 |
| 0.5 | 300 | 70 | 0.0337 | 87 |
| 0.1 | 267 | 35 | 0.0337 | 97 |
| 0.1 | 300 | 35 | 0.0337 | 109 |
| 0.5 | 500 | 70 | 0.0337 | 145 |
| 0.25 | 1065 | 35 | 0.0337 | 155 |
| 0.05 | 267 | 35 | 0.0413 | 158 |
| 0.25 | 300 | 70 | 0.0337 | 174 |
| 0.05 | 300 | 35 | 0.0413 | 178 |
| 0.5 | 500 | 70 | 0.026 | 188 |
| 0.1 | 500 | 35 | 0.0337 | 182 |
| 0.5 | 2500 | 35 | 0.0337 | 182 |
| 0.05 | 267 | 35 | 0.0337 | 194 |
| 0.1 | 533 | 35 | 0.0337 | 194 |
| 0.05 | 300 | 35 | 0.0337 | 218 |
| 0.05 | 267 | 35 | 0.026 | 252 |
| 0.05 | 300 | 35 | 0.026 | 283 |
| 0.25 | 500 | 70 | 0.0337 | 291 |
| 0.05 | 500 | 35 | 0.0413 | 297 |
| 0.05 | 533 | 35 | 0.0413 | 316 |
| 0.05 | 267 | 45 | 0.0337 | 321 |
| 0.05 | 300 | 45 | 0.0337 | 361 |
| 0.05 | 500 | 35 | 0.0337 | 364 |
| 0.25 | 2500 | 35 | 0.0337 | 364 |
| 0.5 | 5000 | 35 | 0.0337 | 364 |
| 0.25 | 500 | 70 | 0.026 | 377 |
| 0.05 | 533 | 35 | 0.0337 | 387 |
| 0.1 | 1065 | 35 | 0.0337 | 387 |
| 0.05 | 500 | 35 | 0.026 | 471 |
| 0.1 | 300 | 70 | 0.0337 | 436 |
| 0.1 | 300 | 70 | 0.0337 | 436 |
| 0.05 | 533 | 35 | 0.026 | 502 |
| 0.1 | 1065 | 35 | 0.026 | 502 |
| 0.25 | 5000 | 35 | 0.0413 | 593 |
| 0.5 | 10000 | 35 | 0.0413 | 593 |
| 0.05 | 500 | 45 | 0.0337 | 601 |
| 0.05 | 500 | 45 | 0.0337 | 601 |
| 0.05 | 533 | 45 | 0.0337 | 641 |
| 0.1 | 500 | 70 | 0.0337 | 727 |
| 0.5 | 10000 | 35 | 0.0337 | 727 |
| 0.5 | 2500 | 70 | 0.0337 | 727 |
| 0.1 | 2500 | 35 | 0.0413 | 742 |
| 0.05 | 1065 | 35 | 0.0337 | 774 |
| 0.05 | 1065 | 35 | 0.0337 | 774 |
| 0.05 | 267 | 70 | 0.0337 | 776 |
| 0.05 | 300 | 70 | 0.0337 | 872 |
| 0.05 | 1065 | 35 | 0.026 | 1004 |
| 0.05 | 500 | 70 | 0.0413 | 1186 |
| 0.05 | 408 | 70 | 0.0337 | 1186 |

Additional aspects of the disclosure are set forth in the Embodiments E1-E48 as well as the embodiments that follow.

E1. A Sustained Release Formulation (SRF) for treating BPH by dispensing no more than a unit volume of the SRF at a location in the prostate using a needle syringe, comprising: a cytostatic or cytotoxic drug; a glycolide-based bioabsorbable copolymer; and a water soluble solvent capable of dissolving the drug and copolymer.

E2. The SRF of E1 or any embodiments depending from E1, wherein the glycolide-based bioabsorbable copolymer is selected from the set of poly(D,L-lactide-co-glycolide) (50:50), poly(D,L-lactide-co-glycolide) (65:35), and poly(D,L-lactide-co-glycolide) (85:15).

E3. The SRF of E1 or any embodiments depending from E1, wherein the water soluble solvent capable of dissolving the drug and copolymer is selected from the set of N-methylpyrrolidone (NMP) and dimethyl sulfoxide (DMSO).

E4. The SRF of E1 or any embodiments depending from E1, wherein the cytotoxic drug is selected from the set of paclitaxel, docetaxel, taxanes, protaxel, vincristine, etoposide, nocodazole, indirubin, anthracycline derivatives, daunorubicin, daunomycin, plicamycin, tauromustine, bofumustane, and plicamycin, irinotecan and doxorubicin and combinations thereof.

E6. The SRF of E1 or any embodiments depending from E1, wherein the glycolide-based bioabsorbable copolymer is selected from the set of poly(D,L-lactide-co-glycolide) (PLGA) and PLGA-PEG-PLGA.

E7. The SRF of E1 or any embodiments depending from E1, wherein glycolide-based bioabsorbable copolymer has a total concentration of 25-75% by wt., the solvent has a total concentration of 75-25% by wt., and the cytotoxic or cytostatic drug has a total concentration of 0.5%-30% by wt.; 1%-20% by wt. o, or 2%-6% by wt.

E8. The SRF of E1 or any embodiments depending from E1, wherein the composition is adapted to fully release the drug into the prostate over at least 14 days from being injected into the prostate, or over a 30 to 90 day period, or over a 90 to 180 day period.

E9. The SRF of E1 or any embodiments depending from E1, wherein the drug has a release rate of no more than 10% to 85% over the first month, 25% to 95% over the first three months, and/or 50 wt. % to 100% over the first six months.

E10. The SRF of E1 or any embodiments depending from E1, wherein the drug has a release rate of between 5% to 50 wt. % during the first 24 hours from injecting the composition into the prostate.

E11. The SRF of E1 or any embodiments depending from E1, wherein the glycolide-based bioabsorbable copolymer has a viscosity of between 0.2-0.5 dL/g, or 0.2 to 0.4 dL/g or 0.2 to 0.3 dL/g and the ratio of DL-lactide to glycolide is from 50/50 up to 85/15.

E12. The SRF of E1 or any embodiments depending from E1, wherein the cytostatic or cytotoxic drug is 0.1 up to 10% wt., 1-15% or up 20-30% wt. of the composition.

E13. The SRF of E1 or any embodiments depending from E1, wherein the solvent comprises N-methyl-pyrrolidone, the drug is paclitaxel, and the polymer is PLGA.

E14. The SRF of E1 or any embodiments depending from E1, wherein the SRF is adapted to release the drug into the prostate over at least 14 days from being injected into the prostate, or over a 30 to 90 day period, or over a 90 to 180 day period.

E15. The SRF of E1 or any embodiments depending from E1, wherein the drug is a cytostatic drug consisting of rapamycin, sirolimus, everolimus, temsirolimus, or zotarolimus.

E16. The SRF of E1 or any embodiments depending from E1, where the drug is a cytotoxic drug comprising paclitaxel or docetaxel.

E17. The SRF of E1 or any embodiments depending from E1, wherein the SRF forms into a solid material with shapes of one or more of, or any of combination of microparticles, nanoparticles, rods, or a gel when the SRF comes into contact with the prostate.

E18. An apparatus for treating a prostate volume, PV, comprising: a composition comprising an SRF having an absolute viscosity, p, and in an amount of at least one unit volume, v; and a syringe containing the composition in a barrel of the syringe, the syringe including a needle having a length, L, and lumen diameter, D, and adapted for being placed in fluid communication with the composition; wherein the syringe holds at least n of the unit volumes, v, n is related to PV as n=PV/(200*v) and the n unit volumes equal a total volume for treating BPH; and wherein the apparatus has a KIR between 10 and 1000, 10 and 300, or 40 and 400, wherein the KIR is defined as (μ)*(L)\hat{ }2/(vD)*10\hat{ }(−6).

E19. The apparatus of E18 or any claim depending therefrom, wherein p is between 500 cP and 6000 cP.

E19b. The apparatus of E18 or any claim depending therefrom, wherein p is between 200 cP and 500 cP.

E20. The apparatus of E18 or any claim depending therefrom, wherein L is between 10 and 20 cm.

E21. The apparatus of E18 or any claim depending therefrom, wherein the barrel has a slenderness ratio of between 5 and 50, or more narrowly between 10 and 30.

E22. The apparatus of E18 or any claim depending therefrom, wherein the needle has a slenderness ratio (L/D) of between 200 and 400, or between 250 and 350.

E23. The apparatus of E18 or any claim depending therefrom, wherein the needle has an inner diameter of 0.02 cm to about 0.12 cm or more narrowly 0.06 cm to 0.08 cm.

E24. The apparatus of E18 or any claim depending therefrom, wherein v is between 0.05 ml and 0.2 ml.

E25. The apparatus of E18 or any claim depending therefrom, wherein L is between 15 and 20 cm, the barrel has a slenderness ratio of about 10 and 30, the needle has an inner diameter of 0.06 cm to about 0.08 cm, v is between 0.05 ml and 0.2 ml, and the viscosity is between 1065 cP and 4000 cP.

E26. The apparatus of E18 or any claim depending therefrom, wherein the syringe barrel contains between 2 and 10 unit volumes of the composition, excluding dead volume of composition within the syringe barrel.

E27. The apparatus of E18 or any claim depending therefrom, wherein the composition comprises a bioabsorbable polymer at a total concentration of 40-50% by wt., a solvent at a total concentration of 80-20%, 75-25%, 60-40%, by wt., and a drug at a total concentration of 0.5%-30% by wt.; 1%-20% by wt. o, or 2%-6% by wt.

E28. The apparatus of E18 or any claim depending therefrom, wherein a drug of the SRF has a release rate of no more than 10% to 85% over the first month, 25% to 95% over the first three months, and/or 50% to 100% over the first six months.

E29. The apparatus of E18 or any claim depending therefrom, wherein the drug has a release rate of between 5% to 50% during the first 24 hours from injecting the composition into the prostate.

E30. The apparatus of E18 or any claim depending therefrom, wherein the SRF comprises a cytostatic or cytotoxic drug of 0.1 up to 10% wt., 10-15% or up to 20-30% wt. of the SRF.

E31. The apparatus of E18 or any claim depending therefrom, wherein the drug is sirolimus, docetaxel, or paclitaxel.

E32. The apparatus of E18 or any claim depending therefrom, wherein the drug comprises an alpha blocker or 5-alpha reductase inhibitor, anti-inflammatory such as corticosteroids, and/or vasodilators.

E33. The apparatus of E18 or any claim depending therefrom, wherein the solvent comprises N-methyl-pyrrolidone, the drug is paclitaxel, and the polymer is PLGA.

E34. The apparatus of E18 or any claim depending therefrom, wherein the KIR is between 40 and 400.

E35. An apparatus for treating a prostate volume, PV, comprising a composition comprising an SRF having an absolute viscosity, p, and in an amount of at least one unit volume, v; a syringe containing the composition in a barrel of the syringe, the syringe including a needle having a length, L, and lumen diameter, D, and adapted for being placed in fluid communication with the composition; and wherein the syringe holds at least n of the unit volumes and n is related to PV as n=PV/(200*v); and wherein L is between 15 and 20 cm, the barrel has a slenderness ratio of about 10 and 30, the needle has an inner diameter of 0.06 cm to about 0.08 cm, v is between 0.05 ml and 0.1 ml, and p is between 1065 cP and 4000 cP; and wherein the apparatus has a KIR between 10 and 1000, defined as (p)*(L)\hat{ }2/(vD)*10\hat{ }(−6).

E36. The apparatus Claim 18, wherein the KIR is between 40 and 400.

E37. The apparatus Claim 18, wherein the syringe barrel contains between 2 and 10 unit volumes of the composition, excluding dead volume of composition within the syringe barrel.

E38. Method of making, comprising: combining a cytotoxic or cytostatic drug, a glycolide-based bioabsorbable copolymer, and a water soluble solvent capable of dissolving the drug and copolymer to form composition for treating BPH;

E39. The method of making according to E48 or any claim depending therefrom, wherein the composition when placed within a needle syringe forms an apparatus having a KIR of between 10 and 1000.

E.40. The method of making according to E48 or any claim depending therefrom, wherein the composition when delivered to a prostate produces an efficacious outcome when delivered to a prostate as a total volume.

E.41. The method of making according to E48 or any claim depending therefrom, wherein the cytotoxic or cytostatic drug, the glycolide-based bioabsorbable copolymer, and the water soluble solvent are combined when placed within a barrel of the needle syringe.

In some embodiments, including any of the foregoing, KIR is from 10 to 300; from 100 to 900; from 300 to 600; from 10 to 1000; or from 40 to 400.

In some embodiments, including any of the foregoing, TZUL is from 1.5 cm to 3.5 cm.

In some embodiments, including any of the foregoing, TZUL is 2 cm, v is 0.05 ml, and n is 3.

In some embodiments, including any of the foregoing, n is 1 to 10.

In some embodiments, including any of the foregoing, n is 1, 2, 3, 4, 5, 6, 7, 8, 9, or 10.

In some embodiments, including any of the foregoing, n is 2 to 6.

In some embodiments, including any of the foregoing, the n number of unit volumes, v, of the composition is such that n=TZUL/(11*v).

In some embodiments, including any of the foregoing, the n number of unit volumes, v, of the composition is such that n is related to prostate volume, PV, as n=PV/(200*v).

In some embodiments, including any of the foregoing, KIR is from 10 to 300; from 100 to 900; from 300 to 600; from 10 to 1000; or from 40 to 400.

In some embodiments, including any of the foregoing, the needle syringe has a needle slenderness ratio greater than 300.

In some embodiments, including any of the foregoing, the needle syringe has a needle slenderness ratio from 300 to 1100.

In some embodiments, including any of the foregoing, the needle is longer than 5 mm.

In some embodiments, including any of the foregoing, the needle can turn 90°.

The unit volume delivery system of any one of claims 11-35, comprising a handle.

The unit volume delivery system of any one of claims 11-35, configured as an injection gun.

In some embodiments, including any of the foregoing, also included is dead volume control.

In some embodiments, including any of the foregoing, p is from 1065 cP to 4000 cP.

In some embodiments, including any of the foregoing, L is from 10 to 20 cm.

In some embodiments, including any of the foregoing, L is from 15 to 20 cm.

In some embodiments, including any of the foregoing, the needle has a slenderness ratio (LID) from 250 to 350.

In some embodiments, including any of the foregoing, the needle has an inner diameter of 0 0.06 cm to 0.08 cm.

In some embodiments, including any of the foregoing, the syringe barrel contains 2 to 10 unit volumes of the composition, excluding dead volume of composition within the syringe barrel.

In some embodiments, including any of the foregoing, the composition has a release rate of no more than 10% to 85% over the first month.

In some embodiments, including any of the foregoing, the composition has a release rate of from 5% to 50% during the first 24 hours from injecting the composition into the prostate.

In some embodiments, including any of the foregoing, the composition comprises 0.1 up to 10% wt. of sirolimus or paclitaxel.

In some embodiments, including any of the foregoing, the composition comprises an alpha blocker, a 5-alpha reductase inhibitor, an anti-inflammatory, a vasodilator, or a combination thereof.

In some embodiments, including any of the foregoing, the concentration of the glycolide-based bioabsorbable copolymer in the composition is 40 to 50% by wt.; the concentration of the solvent is 20 to 80% by wt.; and the concentration of the drug is 0.5% to 30% by wt.;

In some embodiments, including any of the foregoing, the bioabsorbable polymer is poly(lactide-co-glycolide) (PLGA).

In some embodiments, including any of the foregoing, the solvent is N-methyl-pyrrolidone.

In some embodiments, including any of the foregoing, drug is sirolimus or paclitaxel.

In some embodiments, including any of the foregoing,
PV is 20 to 80 cm³,
L is between 15 and 20 cm,
the barrel has a slenderness ratio of about 200 to 400,
D is about 0.06 cm to about 0.08 cm,
v is between 0.05 ml and 0.1 ml, and
μ is between 1065 cP and 4000 cP.

In some embodiments, including any of the foregoing,
PV is 20 to 200 cm³,
L is between 15 and 20 cm,
the barrel has a slenderness ratio of about 200 to 400,
D is about 0.06 cm to about 0.08 cm,
v is between 0.05 ml and 0.1 ml, and
μ is between 1065 cP and 4000 cP.

In some embodiments, including any of the foregoing, the needle syringe comprises a needle capable of creating multiple lesions along the urethra transition zone.

In some embodiments, including any of the foregoing, the needle syringe comprises up to twenty side holes through which the needle syringe is capable of injecting the composition.

In some embodiments, including any of the foregoing, the needle syringe comprises 10 to 20 side holes through which the needle syringe is capable of injecting the composition.

In some embodiments, including any of the foregoing, the needle syringe comprises 12 side holes through which the needle syringe is capable of injecting the composition.

In some embodiments, including any of the foregoing, the needle syringe is 22 cm long with large 20 Fr/6.7 mm OD.

In some embodiments, including any of the foregoing, the needle is flexible.

In some embodiments, including any of the foregoing, the needle is rigid.

The unit volume delivery system of any one of claims 11-66, further comprising a luer port to connect to the needle.

The unit volume delivery system of any one of claims 11-67, configured to be opened for manual retrieval of the needle.

In some embodiments, including any of the foregoing, the needle length is greater than 5 mm.

In some embodiments, including any of the foregoing, the needle length is greater about 8 mm.

In some embodiments, including any of the foregoing, the needle is a transrectal needle.

In some embodiments, including any of the foregoing, the needle is a transurethral needle.

In some embodiments, including any of the foregoing, also included is a 18 G needle.

In some embodiments, including any of the foregoing, also included is a 20 G needle.

In some embodiments, including any of the foregoing, also included is a needle smaller than a 20 G needle.

In some embodiments, including any of the foregoing, also included is a 22 G needle.

In some embodiments, including any of the foregoing, also included is a 23 G needle.

In some embodiments, including any of the foregoing, also included is a 25 G needle.

The unit volume delivery system of any one of claims 11-72, wherein the needle is 1 mm to 35 cm in length.

In some embodiments, including any of the foregoing, the needle is 4 mm to 20 cm in length.

In some embodiments, including any of the foregoing, also included is a transrectal 20 G×20 cm needle.

In some embodiments, including any of the foregoing, also included is a transrectal 20 G×20 cm needle.

In some embodiments, including any of the foregoing, the needle is rigid.

In some embodiments, including any of the foregoing, the needle is flexible.

The unit volume delivery system of any one of claims 11-72, comprising a Cook 45 cm long needle.

The unit volume delivery system of any one of claims 11-72, comprising a 23 G 8 mm long needle.

In some embodiments, including any of the foregoing, also included is a Storrs, Olympus or Ambu flexible cystoscopes.

In some embodiments, including any of the foregoing, also included is a flexible scope.

In some embodiments, including any of the foregoing, also included is a hollow plastic handle with buttons configured to couple to an optical rigid cystoscope.

In some embodiments, including any of the foregoing, also included is a long slender transurethral needle that has a curved tip to access the urethra.

In some embodiments, including any of the foregoing, also included is a on the proximal side a luer port for the needle.

In some embodiments, including any of the foregoing, also included is a plastic syringe support to hold the syringe fixed in place with the handle while still allowing rotation.

In some embodiments, including any of the foregoing, the needle is rotatable.

In some embodiments, including any of the foregoing, the needle is rotatable via the syringe and luer port for additional injections.

In some embodiments, including any of the foregoing, the unit volume delivery system is made from injection molding.

In some embodiments, including any of the foregoing, also included is a long flexible needle placed inside the fluid port of a commercial flexible cystoscope.

In some embodiments, including any of the foregoing, also included is a curved needle tip of 5 mm or more length.

In some embodiments, including any of the foregoing, also included is a curved needle tip of 10 mm or more length.

In some embodiments, including any of the foregoing, also included is a collar coupled to the plunger of the needle syringe, where: the collar is configured to dispense a single unit volume.

In some embodiments, including any of the foregoing, also included is a collar coupled to the plunger of the needle syringe, where: the collar is configured to dispense a total volume.

In some embodiments, including any of the foregoing, also included is a ratcheting system that engages the plunger for each delivered unit volume.

In some embodiments, including any of the foregoing, set forth herein is a method for treating Benign Prostatic Hyperplasia (BPH) in a subject in need thereof, comprising:

providing the unit volume delivery system set forth herein, and dispensing, with the unit volume delivery system, a total volume of the composition into a prostate to produce an efficacious outcome for treating BPH, including dispensing no more than a unit volume of the composition into a prostate at a first prostate location using the needle syringe.

In some embodiments, including any of the foregoing, the method includes the step of placing the needle syringe at a second location within the prostate and dispensing no more than one unit volume of the composition into the prostate at the second location.

In some embodiments, including any of the foregoing, the method includes a plurality, n, of the no more than a unit volume, v, injected into the prostate to treat the prostate at a corresponding n prostate locations, wherein n=TZUL/(11*v).

In some embodiments, including any of the foregoing, the method includes a plurality, n, of the no more than a unit volume, v, injected into the prostate to treat the prostate at a corresponding n prostate locations, wherein n is related to prostate volume, PV, as n=PV/(200*v), wherein the sum of the unit volumes is a total volume for the prostate volume, PV.

In some embodiments, including any of the foregoing, the needle syringe includes a barrel that holds at least n of the unit volumes, wherein n is an integer from 2 to 10; wherein:
n is 2 to 4 when a prostate size is between 20 and 40 grams,
n is 2 to 6 when the prostate size is between 40 and 60 grams,
n is 4 to 8 when the prostate size is between 60 and 80 grams, and
n is 6 to 10 when the prostate size is 80 grams or more.

In some embodiments, including any of the foregoing, unit volume is 0.05 ml to 0.5 ml, 0.05 ml to 0.1 ml, 0.1 to 0.2 ml, or 0.05 to 0.2 ml.

In some embodiments, including any of the foregoing, composition has between 4% to 15% vol. of the cytotoxic or cytostatic drug.

In some embodiments, including any of the foregoing, the cytotoxic drug or cytostatic drug is selected from the group consisting of paclitaxel, sirolimus, docetaxel, everolimus, carmustine, mitomycin, tacrolimus, and temsirolimus.

In some embodiments, including any of the foregoing, the cytotoxic drug is docetaxel or paclitaxel.

In some embodiments, including any of the foregoing, the cytostatic drug is sirolimus.

In some embodiments, including any of the foregoing, also included is the step of measuring the TZUL and based on the TZUL, selecting a total number of unit volumes, v, of the composition to dispense into the prostate at a respective plurality of locations of the prostate, the sum of the unit volumes being about equal to the total volume.

In some embodiments, including any of the foregoing, also included is the step of measuring prostate size and based on the prostate size, selecting a total volume of the composition for treating BPH including selecting the number of unit volumes of the composition to dispense into the prostate at a respective plurality of locations of the prostate, the sum of the unit volumes being about equal to the total volume.

In some embodiments, including any of the foregoing, the prostate size is a prostate volume or a prostrate weight, and wherein the prostate volume or prostate weight is measured by ultrasonic imaging.

In some embodiments, including any of the foregoing, the composition further comprises an alpha blocker or 5-alpha reductase inhibitor or an anti-inflammatory.

In some embodiments, including any of the foregoing, the glycolide-based bioabsorbable copolymer is selected from the set of poly(D,L-lactide-co-glycolide) wherein the molar ratio of lactide to glycolide is 50:50, poly(D,L-lactide-co-glycolide) wherein the molar ratio of lactide to glycolide is 65:35, and poly(D,L-lactide-co-glycolide) wherein the molar ratio of lactide to glycolide is 75:25, and poly(D,L-lactide-co-glycolide) wherein the molar ratio of lactide to glycolide is 85:15.

In some embodiments, including any of the foregoing, the water soluble solvent capable of dissolving the drug and copolymer is selected from the set of N-methylpyrrolidone (NMP) and dimethyl sulfoxide (DMSO), and combinations thereof.

In some embodiments, including any of the foregoing, the glycolide-based bioabsorbable copolymer is selected from the set of poly(D,L-lactide-co-glycolide) (PLGA) and PLGA-PEG-PLGA.

In some embodiments, including any of the foregoing, glycolide-based bioabsorbable copolymer has a total concentration of 30-50% by wt., the solvent has a total concentration of 50-30% by wt., and the cytotoxic or cytostatic drug has a total concentration of 0.5%-30% by wt.; 1%-20% by wt.; or 2%-6% by wt.

In some embodiments, including any of the foregoing, the drug has a release rate of between 5% to 50% during the first 24 hours from injecting the composition into the prostate.

In some embodiments, including any of the foregoing, the solvent comprises N-methyl-pyrrolidone, the drug is paclitaxel and the polymer is PLGA.

In some embodiments, including any of the foregoing, a unit volume is from 0.05 ml to 0.2 ml.

In some embodiments, including any of the foregoing, the plurality of unit volumes is equal to a total volume for treating BPH.

In some embodiments, including any of the foregoing, the composition has between 4% to 15% vol. of the cytotoxic or cytostatic drug, wherein the cytotoxic drug is docetaxel or paclitaxel, and wherein the cytostatic drug is sirolimus.

In some embodiments, including any of the foregoing, the n number of unit volumes of the composition, wherein n is related to TZUL as n=TZUL/(11*v).

In some embodiments, including any of the foregoing, the plurality of unit volumes, n, is related to a prostate volume, PV, for treatment of BPH as n=PV/(200*v).

In some embodiments, including any of the foregoing, the anti-inflammatory is selected from corticosteroids, vasodilators, and combinations thereof.

In some embodiments, including any of the foregoing, the following active agents may be administered in combination the methods, devices, and apparatus set forth herein.

In some embodiments, including any of the foregoing, also included are analgesic or pain medications, such as, but not limited to: Lidocaine, Xylocaine, Buvipocaine, bupivacaine, cocaine, ropivacaine, mepivacaine, etidocaine, levobupivacaine, trimecaine, carticaine, articaine, lidocaine, prilocaine, benzocaine, procaine, tetracaine, chloroprocaine, and combinations thereof.

In some embodiments, including any of the foregoing, also included are 5-alpha reductase inhibitors such as, but not limited to: Dutasteride and Finasteride.

In some embodiments, including any of the foregoing, also included are alpha blockers and smooth muscle cell relaxers such as, but not limited to: doxazosin, prazosin, terazosin, tamsulosin, alfuzosin, silodosin, phenoxybenzamine, vibegron, beta 3 adregenic receptor agonists, mirabegron, and tolterodine.

In some embodiments, including any of the foregoing, also included are Anti-inflammatories such as, but not limited to: Fluticaosone furoate, mometasone furoate, dexamethasone, prednisone, betamethasone, cortisone, hydrocortisone, methylprednisolone, NSAIDs, aspirin, ibuprofen, naproxen sodium, diclofenac, diclofenac-misoprostol, celexocib, piroxicam, indomethacin meloxicam, ketoprofen, sulindac, diflunisal, nabumetone, oxaprozin, tolmetin, salsalate, etodolac, fenoprofen, flurbiprofen, ketorolac, meclofenamate, mefenamic acid, and other COX-2 inhibitors, and combinations thereof.

In some embodiments, including any of the foregoing, also included are anti-cholinergics such as, but not limited to: atropine, belladonna alkaloids, benztropine mesylate, clidinium, cyclopentolate, darifenacin, dicylomine, diphenhydramine, fesoteroidine, flavoxate, glycopyrrolate, homatropine hydrobromide, hyoscyamine, ipratropium, orphenadrine, oxybutynin, propantheline, scopolamine, methscopolamine, solifenacin, succinylcholine, tiotropium, tolterodine, trihexyphenidyl, trospium, vecuronium.

In some embodiments, including any of the foregoing, also included are anti-fungals, anti-microbials, antibiotics, and antimicrobials such as, but not limited to: amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, .alpha.-defensins, and .alpha.-protegrins.

Antifungal agents, in some embodiments, include, but are not limited to, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, and amphotericin B.

In some embodiments, including any of the foregoing, also included are antimuscarinics such as, but not limited to: tolterodine, solifenacin, darifenacin, propiverine, oxybutynin, trospium, and chloride.

In some embodiments, including any of the foregoing, also included are others drugs such as, but not limited to: Capsaicin, and botox.

In some embodiments, including any of the foregoing, single drug may be utilized.

In some embodiments, including any of the foregoing, combinations of various drugs may be utilized.

In some embodiments, including any of the foregoing, also included are drugs such as enzalutamide (non-steroidal antiandrogen) and abiraterone acetate (Zytidga).

In some embodiments, set forth herein is a method for treating Benign Prostatic Hyperplasia (BPH) in a subject in need thereof, comprising:
using a needle syringe containing a composition comprising a cytotoxic or cytostatic drug, a glycolide-based bioabsorbable copolymer, and a water soluble solvent capable of dissolving the drug and copolymer; and
dispensing a total volume of the composition into a prostate to produce an efficacious outcome for treating BPH, including dispensing no more than a unit volume of the composition into a prostate at a first prostate location using the needle syringe.

In some embodiments, including any of the foregoing, the methods comprise using an apparatus comprising the needle syringe and the composition, the apparatus having a KIR of between 10 and 1000, 10 and 300, or 40 and 400.

In some embodiments, including any of the foregoing, the step of placing the needle syringe at a second location within the prostate and dispensing no more than one unit volume of the composition into the prostate at the second location using the apparatus.

The method of any one of claims 1-3, wherein a plurality, n, of the no more than a unit volume, v, are injected into the prostate to treat the prostate at a corresponding n prostate locations, wherein n is related to prostate volume, PV, as n=PV/(200*v), wherein the sum of the plurality of the n of the unit volumes is a total volume for the prostate volume, PV.

In some embodiments, including any of the foregoing, the needle syringe includes a barrel that holds at least n of the unit volumes, such that
n is 2 to 4 when a prostate size is between 20 and 40 grams,
n is 2 to 6 when the prostate size is between 40 and 60 grams,
n is 4 to 8 when the prostate size is between 60 and 80 grams, and
n is 6 to 10 when the prostate size is 80 grams or more.

In some embodiments, including any of the foregoing, a unit volume is 0.05 ml to 0.5 ml, 0.05 ml to 0.1 ml, 0.1 to 0.2 ml, or 0.05 to 0.2 ml.

In some embodiments, including any of the foregoing, the composition has between 4% to 15% vol. of the cytotoxic or cytostatic drug.

In some embodiments, including any of the foregoing, the cytotoxic drug is docetaxel or paclitaxel.

In some embodiments, including any of the foregoing, the cytostatic drug is sirolimus.

In some embodiments, including any of the foregoing, the step of measuring prostate size and based on the prostate size, selecting a total volume of the composition for treating BPH including selecting the number of unit volumes of the composition to dispense into the prostate at a respective plurality of locations of the prostate, the sum of the unit volumes being about equal to the total volume.

In some embodiments, including any of the foregoing, the prostate volume or weight is measured by ultrasonic imaging.

In some embodiments, including any of the foregoing, the composition further comprises an alpha blocker or 5-alpha reductase inhibitor, anti-inflammatory such as corticosteroids, and/or vasodilators.

In some embodiments, including any of the foregoing, the glycolide-based bioabsorbable copolymer is selected from the set of poly(D,L-lactide-co-glycolide) (50:50), poly(D,L-lactide-co-glycolide) (65:35), and poly(D,L-lactide-co-glycolide) (75:25), and poly(D,L-lactide-co-glycolide) (85:15).

In some embodiments, including any of the foregoing, the water soluble solvent capable of dissolving the drug and copolymer is selected from the set of N-methylpyrrolidone (NMP) and dimethyl sulfoxide (DMSO), and combinations thereof.

In some embodiments, including any of the foregoing, the glycolide-based bioabsorbable copolymer is selected from the set of poly(D,L-lactide-co-glycolide) (PLGA) and PLGA-PEG-PLGA.

In some embodiments, including any of the foregoing, the glycolide-based bioabsorbable copolymer has a total concentration of 30-50% by wt., the solvent has a total concentration of 50-30% by wt., and the cytotoxic or cytostatic drug has a total concentration of 0.5%-30% by wt.; 1%-20% by wt. o, or 2%-6% by wt.

In some embodiments, including any of the foregoing, the drug has a release rate of between 5% to 50% during the first 24 hours from injecting the composition into the prostate.

In some embodiments, including any of the foregoing, the solvent comprises N-methyl-pyrrolidone, the drug is paclitaxel, and the polymer is PLGA.

In some embodiments, set forth herein is a method of treating Benign Prostatic Hyperplasia (BPH) in a subject in need thereof, comprising
using an apparatus comprising a needle syringe containing a composition comprising a cytotoxic or cytostatic drug, a glycolide-based bioabsorbable copolymer, and a water soluble solvent capable of dissolving the drug and copolymer, wherein the composition has an absolute viscosity, p, and the needle syringe has a needle length L with inner diameter D; and
dispensing a plurality of unit volumes, v, of the composition at a respective plurality of different locations in the prostate using the needle syringe;
wherein a unit volume is from 0.05 ml to 0.2 ml;
wherein the plurality of unit volumes is equal to a total volume for treating BPH; and
wherein the apparatus has a KIR of between 10 to 300 or 40 to 400 and KIR is defined as

$$KIR = \frac{μ {L}^{2}}{vD} ( 1 {0}^{- 6} ) .$$

In some embodiments, including any of the foregoing, the composition has between 4% to 15% vol. of the cytotoxic or cytostatic drug, wherein the cytotoxic drug is docetaxel or paclitaxel, and wherein the cytostatic drug is sirolimus.

In some embodiments, including any of the foregoing, the plurality of unit volumes, n, is related to a prostate volume, PV, for treatment of BPH as n=PV/(200*v).

In some embodiments, set forth herein is an apparatus for treating a prostate volume, PV, comprising:
a composition comprising an SRF having an absolute viscosity, p, and in an amount of at least one unit volume, v; and
a syringe containing the composition in a barrel of the syringe, the syringe including a needle having a length, L, and lumen diameter, D, and adapted for being placed in fluid communication with the composition;
wherein the syringe holds at least n of the unit volumes, v, n is related to PV as n=PV/(200*v) and the n unit volumes equal a total volume for treating BPH; and
wherein the apparatus has a KIR between 10 and 1000, 10 and 300, or 40 and 400, wherein the KIR is defined as (p)*(L)\hat{ }2/(vD)*10\hat{ }(−6).

In some embodiments, including any of the foregoing, p is between 500 cP and 6000 cP.

In some embodiments, including any of the foregoing, the L is between 10 and 20 cm.

In some embodiments, including any of the foregoing, the barrel has a slenderness ratio of between 5 and 50, or more narrowly between 10 and 30.

In some embodiments, including any of the foregoing, the needle has a slenderness ratio (L/D) of between 200 and 400, or between 250 and 350.

In some embodiments, including any of the foregoing, the needle has an inner diameter of 0.02 cm to about 0.12 cm or more narrowly 0.06 cm to 0.08 cm.

In some embodiments, including any of the foregoing, the v is between 0.05 ml and 0.2 ml.

In some embodiments, including any of the foregoing, the L is between 15 and 20 cm,
the barrel has a slenderness ratio of about 10 and 30,
the needle has an inner diameter of 0.06 cm to about 0.08 cm,
v is between 0.05 ml and 0.2 ml, and
the viscosity is between 1065 cP and 4000 cP.

In some embodiments, including any of the foregoing, the syringe barrel contains between 2 and 10 unit volumes of the composition, excluding dead volume of composition within the syringe barrel.

In some embodiments, including any of the foregoing, the composition comprises
a bioabsorbable polymer at a total concentration of 40-50% by wt., a solvent at a total concentration of 80-20%, 75-25%, 60-40%, by wt., and a drug at a total concentration of 0.5%-30% by wt.; 1%-20% by wt. o, or 2%-6% by wt.

In some embodiments, including any of the foregoing, a drug of the SRF has a release rate of no more than 10% to 85% over the first month, 25% to 95% over the first three months, and/or 50% to 100% over the first six months.

In some embodiments, including any of the foregoing, the drug has a release rate of between 5% to 50% during the first 24 hours from injecting the composition into the prostate.

In some embodiments, including any of the foregoing, the SRF comprises a cytostatic or cytotoxic drug of 0.1 up to 10% wt., 10-15% or up to 20-30% wt. of the SRF.

In some embodiments, including any of the foregoing, the drug is sirolimus, docetaxel, or paclitaxel.

In some embodiments, including any of the foregoing, the drug comprises an alpha blocker or 5-alpha reductase inhibitor, anti-inflammatory such as corticosteroids, and/or vasodilators.

In some embodiments, including any of the foregoing, the solvent comprises N-methyl-pyrrolidone, the drug is paclitaxel and the polymer is PLGA.

In some embodiments, including any of the foregoing, the KIR is between 40 and 400.

In some embodiments, set forth herein is an apparatus for treating a prostate volume, PV, comprising
a composition comprising an SRF having an absolute viscosity, p, and in an amount of at least one unit volume, v;
a syringe containing the composition in a barrel of the syringe, the syringe including a needle having a length, L, and lumen diameter, D, and adapted for being placed in fluid communication with the composition; and
wherein the syringe holds at least n of the unit volumes and n is related to PV as n=PV/(200*v); and
wherein
   L is between 15 and 20 cm,
   the barrel has a slenderness ratio of about 10 and 30,
   the needle has an inner diameter of 0.06 cm to about 0.08 cm,
   v is between 0.05 ml and 0.1 ml, and
   μ is between 1065 cP and 4000 cP; and
wherein the apparatus has a KIR between 10 and 1000, defined as (p)*(L)\hat{ }2/(vD)*10\hat{ }(−6).

In some embodiments, including any of the foregoing, the KIR is between 40 and 400.

In some embodiments, including any of the foregoing, the syringe barrel contains between 2 and 10 unit volumes of the composition, excluding dead volume of composition within the syringe barrel.

In some embodiments, set forth herein is a guide for providing incremental dosages from a syringe coupled to the guide, the syringe including a barrel, a finger rest extending from the barrel, and a plunger that is movable relative to the barrel, the guide comprising:
(a) a main body extending along a central axis between a first end and a second end, the main body defining an interior space, the main body comprising:
   (1) an exterior wall;
   (2) an interior wall disposed radially inward of the exterior wall; and
   (3) a plurality of detents extending from the interior wall into the interior space, the plurality of detents being spaced apart from one another along the central axis of the main body;
(b) a finger rest retaining portion at the second end of the main body, the finger rest retaining portion being configured to engage the finger rest of the syringe to prevent movement of the barrel relative to the main body;
wherein, when the syringe is provided in the guide, the plunger extends into the interior space of the main body, and wherein, when the plunger is moved relative to the barrel, a first detent of the plurality of detents is configured to engage the plunger to resist movement of the plunger relative to the barrel, the plunger being movable past the first detent of the plurality of detents to a second detent of the plurality of detents.

In some embodiments, including any of the foregoing, the finger rest retaining portion is provided as an aperture, the aperture extending through the main body between the exterior wall and the interior wall, and wherein, when the syringe is provided in the guide, the finger rest extends through the aperture and away from the main body.

In some embodiments, including any of the foregoing, the finger rest retaining portion is provided as a wing, the wing extending radially outward from the exterior wall of the main body, and wherein, when the syringe is provided in the guide, the wing at least partially encloses the finger rest.

In some embodiments, including any of the foregoing, the main body includes a first part and a second part, the second part being movable relative to the first part to transition the guide between a first condition and a second condition, the first condition permitting movement of the barrel relative to the main body, the second condition preventing movement of the barrel relative to the main body.

In some embodiments, including any of the foregoing, the first part and the second part are integrally connected by a hinge, and wherein the main body includes a snap connector for maintaining the guide in the second condition.

In some embodiments, including any of the foregoing, the first part and the second part are discrete components, and wherein the main body includes a snap connector for securing the second part to the first part, the guide being in the second condition when the second part is secured to the first part.

In some embodiments, including any of the foregoing, a first distance (LDC) between the first detent and the second detent along the central axis of the main body corresponds to one unit volume (v) of a composition contained within the barrel.

In some embodiments, including any of the foregoing, the barrel of the syringe has an inner diameter (d), and wherein the first distance (LDC) is proportional to the unit volume (v), the first distance LDC being defined as

LDC=[4/(7πd²)]v In some embodiments, including any of the foregoing, the detents are configured to provide haptic feedback to signal to a user that the plunger has moved the first distance.

In some embodiments, including any of the foregoing, the detents are configured to provide audible feedback to signal to a user that the plunger has moved the first distance.

In some embodiments, including any of the foregoing, the plunger includes a head, and wherein the plurality of detents includes a third detent located between the first detent and the second detent, a second distance between the first detent and the third detent along the central axis of the main body and a third distance between the third detent and the first detent along the central axis of the main body both being equal to a thickness of the head; optionally, or the distances being such as to retain the head between detents and if displaced from either upper or lower detent there is a haptic or audible sound indicating that head was displaced from its position between detents.

In some embodiments, including any of the foregoing, the interior wall has a first circumscribed extent, and wherein the plunger includes a head having a second circumscribed extent, the second circumscribed extent being smaller than the first second circumscribed extent.

In some embodiments, including any of the foregoing, the interior wall has a first circumscribed extent, and wherein the plunger includes a head having a second circumscribed extent, the second circumscribed extent being smaller than the first second circumscribed diameter.

The guide according to any one of claims 42-52, wherein resistance of movement of the plunger relative to the barrel is provided by engagement between the detents and the head of the plunger.

In some embodiments, including any of the foregoing, the plunger includes a head, the head having an obround configuration, and wherein a cross section of the interior wall of the main body approximates at least a portion of the obround configuration of the head of the plunger.

In some embodiments, including any of the foregoing, the detents are provided on only the at least a portion of the obround configuration; optionally, only curved portions of the interior wall of the main body.

In some embodiments, including any of the foregoing, the main body includes weakened portions, the weakened portions being configured to allow the main body to deflect away from the plunger head when the plunger head is forcibly moved between detents.

In some embodiments, including any of the foregoing, the weakened portions are provided as slots extending through the main body between the exterior wall and the interior wall, the slots being arranged to weaken the resistance of the main body to outwardly directed pressure applied when the plunger head is forcibly moved between detents.

In some embodiments, including any of the foregoing, the weakened portions are provided as reduced thickness portions that do not extend through the main body.

In some embodiments, including any of the foregoing, the reduced thickness portions extend substantially parallel with the central axis of the main body.

In some embodiments, including any of the foregoing, the main body includes a cutout portion, the cutout portion extending from a terminal end of the main body toward the first end, the cutout portion being configured to provide clearance for a finger of a user that is manipulating the syringe to move the plunger head towards the barrel when dispensing a unit volume.

In some embodiments, including any of the foregoing, the syringe is provided in the guide, a portion of the barrel of the syringe is disposed in the main body.

In some embodiments, including any of the foregoing, the main body further comprises a plurality of bumpers extending from the interior wall into the interior space, the bumpers being configured to engage the portion of the barrel of the syringe disposed in the main body to prevent movement of the barrel relative to the main body.

In some embodiments, including any of the foregoing, the guide is a unitary assembly that is securable to a top of a syringe barrel of the needle syringe assembly.

In some embodiments, including any of the foregoing, the guide comprises a plurality of n number of unit volumes, v, of a composition, wherein n is related to TZUL as n=TZUL/(11*v); and wherein the composition comprises a cytotoxic drug or cytostatic drug powder, a glycolide-based bioabsorbable copolymer, and a water soluble solvent.

In some embodiments, including any of the foregoing, the guide comprises a plurality of n number of unit volumes of a composition, wherein n is related to prostate volume, PV, as n=PV/(200*v); wherein the composition comprises a cytotoxic drug or cytostatic drug powder, a glycolide-based bioabsorbable copolymer, and a water soluble solvent.

In some embodiments, including any of the foregoing, the TZUL is from 1.5 cm to 3.5 cm.

In some embodiments, including any of the foregoing, the TZUL is 2 cm, v is 0.05 ml, and n is 3.

In some embodiments, including any of the foregoing, the guide comprises dead volume control.

In some embodiments, set forth herein is a delivery device configured to mix a first composition with a second composition, the delivery device comprising:
a barrel extending between a first end and a second end, the first end of the barrel being adapted for attachment to a needle;
a septum disposed within the barrel, the septum dividing the barrel into a first chamber and a second chamber, the first chamber receiving the first composition, the second chamber receiving the second composition; and
a plunger extending from the second end of the barrel, the plunger being movable relative to the barrel, the plunger being configured to break the septum to allow the first composition to mix with the second composition to form a mixture and force the mixture from the first end of the barrel into the needle.

In some embodiments, including any of the foregoing, the guide comprises a piercing member disposed within the barrel, the piercing member being configured to break the septum.

In some embodiments, including any of the foregoing, the plunger engages the piercing member to cause the piercing member to move relative to the barrel to break the septum.

In some embodiments, including any of the foregoing, the plunger comprises a shaft portion extending between a first end and a second end, and wherein a piston is provided at the first end of the shaft portion, the piston being in sealing engagement with an interior surface of the barrel.

In some embodiments, including any of the foregoing, the piston engages the septum to break the septum.

In some embodiments, including any of the foregoing, the guide comprises a piercing member disposed within the barrel, the piercing member being configured to break the septum, wherein the piston engages the piercing member to cause the piercing member to move relative to the barrel to break the septum.

In some embodiments, including any of the foregoing, a head is provided at the second end of the shaft portion, the head having a first circumscribed diameter and the shaft portion having a second circumscribed diameter, the first circumscribed diameter being larger than the second circumscribed diameter.

In some embodiments, including any of the foregoing, the guide comprises a finger rest, the finger rest extending from an exterior surface of the barrel and extending along a first axis that is transverse to a central axis of the barrel.

In some embodiments, including any of the foregoing, the first composition comprises a cytotoxic drug or cytostatic drug powder, and the second composition comprises a glycolide-based bioabsorbable copolymer and a water soluble solvent.

In some embodiments, including any of the foregoing, the cytotoxic drug or cytostatic drug powder is paclitaxel, sirolimus, docetaxel, everolimus, carmustine, mitomycin, tacrolimus, or temsirolimus.

In some embodiments, including any of the foregoing, the glycolide-based bioabsorbable copolymer and a water soluble solvent is PLGA and N-methyl pyrrolidone (NMP).

In some embodiments, including any of the foregoing, the cytotoxic or cytostatic drug is paclitaxel, the glycolide-based bioabsorbable copolymer is PLGA8515, and the water soluble solvent is NMP.

In some embodiments, set forth herein is a kit comprising the guide of any one of claims 42-70, or the delivery device of any one of claims 71-82, and instructions for use thereof.

In some embodiments, set forth herein is a kit comprising: at least two or more pre-filled syringes; wherein one of the at least two or more pre-filled syringes comprises a composition comprising a cytotoxic drug or cytostatic drug powder, wherein one of the at least two or more pre-filled syringes comprises a composition comprising a glycolide-based bioabsorbable copolymer and a water soluble solvent; and instructions for mixing the pre-filled syringes.

In some embodiments, including any of the foregoing, the instructions for mixing the pre-filled syringes instruct how to provide a needle syringe assembly containing at least one unit volume, v, or more of a composition, wherein the composition comprises: the cytotoxic drug or cytostatic drug, the glycolide-based bioabsorbable copolymer, and the water-soluble solvent; and wherein the needle syringe assembly has a K injectate ratio (KIR) from 15 to 1200; wherein KIR is defined as:

$$KIR = \frac{μ {L}^{2}}{vD} ( 1 {0}^{- 6} )$$

wherein the needle syringe has a needle length, L, and lumen diameter, D; and wherein p is the absolute viscosity of the composition.

In some embodiments, including any of the foregoing, the one of the cytotoxic drug or cytostatic drug is selected from paclitaxel, sirolimus, docetaxel, everolimus, carmustine, mitomycin, tacrolimus, or temsirolimus.

In some embodiments, including any of the foregoing, the glycolide-based bioabsorbable copolymer and a water soluble solvent comprises PLGA and NMP.

In some embodiments, including any of the foregoing, the kit comprises instructions for dispensing at least one or more unit volumes, v, of the composition into a prostate to produce an efficacious outcome for treating BPH, including dispensing no more than a unit volume of the composition into a prostate at a first prostate location using the needle syringe.

In some embodiments, including any of the foregoing, the kit comprises an adapter for joining two of the at least two or more pre-filled syringes.

In some embodiments, including any of the foregoing, wherein one of the at least two or more pre-filled syringes comprises paclitaxel.

In some embodiments, including any of the foregoing, wherein one of the at least two or more pre-filled syringes comprises docetaxel.

In some embodiments, including any of the foregoing, wherein one of the at least two or more pre-filled syringes comprises sirolimus.

In some embodiments, including any of the foregoing, wherein one of the at least two or more pre-filled syringes comprises (NMP) and PLGA.

In some embodiments, including any of the foregoing, wherein the kit comprises a transrectal needle.

In some embodiments, including any of the foregoing, wherein the kit comprises a transurethral needle.

In some embodiments, including any of the foregoing, wherein the kit comprises a 18 G needle.

In some embodiments, including any of the foregoing, wherein the kit comprises a 20 G needle.

In some embodiments, including any of the foregoing, wherein the kit comprises a 22 G needle.

In some embodiments, including any of the foregoing, wherein the kit comprises a 23 G needle.

In some embodiments, including any of the foregoing, wherein the kit comprises a 25 G needle.

In some embodiments, including any of the foregoing, the needle is 10 cm to 70 cm in length.

In some embodiments, including any of the foregoing, the needle is 10 cm to 20 cm in length.

In some embodiments, including any of the foregoing, wherein the kit comprises a transrectal 20 G×20 cm needle.

In some embodiments, including any of the foregoing, wherein the kit comprises a transurethral 23 G×70 cm needle.

In some embodiments, including any of the foregoing, wherein the kit comprises a 23 G×45 cm commercial transurethral needle.

In some embodiments, including any of the foregoing, wherein the kit comprises an adapter for coupling one of the at least two or more pre-filled syringes to a cystoscope.

In some embodiments, including any of the foregoing, wherein the adapter is a hub for coupling one of the at least two or more pre-filled syringes to a cystoscope.

In some embodiments, including any of the foregoing, the adapter is a luer lock that is capable of coupling to a flexible cystoscope.

In some embodiments, including any of the foregoing, wherein the kit comprises a Tuehy-Borst-type value.

In some embodiments, including any of the foregoing, wherein the kit comprises unit doses of 50 μL.

In some embodiments, including any of the foregoing, wherein the kit comprises unit doses of 100 μL.

In some embodiments, including any of the foregoing, wherein the kit comprises paclitaxel, NMP, and PLGA8515.

In some embodiments, including any of the foregoing, wherein the kit comprises 50% PLGA 5002A Polymer Solution in NMP.

In some embodiments, including any of the foregoing, wherein the kit comprises at least one dosing guide for providing incremental dosages from one of the at least two or more pre-filled syringes.

In some embodiments, including any of the foregoing, wherein the kit comprises at least one dosing guide for providing incremental dosages of two to three per patient from one of the at least two or more pre-filled syringes.

In some embodiments, including any of the foregoing, wherein the kit comprises at least one dosing guide for providing incremental dosages of 0.05 mL to 0.1 mL unit volumes from one of the at least two or more pre-filled syringes.

In some embodiments, including any of the foregoing, wherein the kit comprises at least one dosing guide for providing incremental dosages of 0.1 mL to 0.2 mL unit volumes from one of the at least two or more pre-filled syringes.

In some embodiments, including any of the foregoing, wherein the kit comprises at least one dosing guide for providing incremental dosages of four to eight unit volumes from one of the at least two or more pre-filled syringes.

In some embodiments, including any of the foregoing, wherein the KIR is from 15 to 300; from 100 to 900; from 300 to 600; from 15 to 1000; or from 40 to 400.

In some embodiments, including any of the foregoing, wherein the kit comprises at least n number of unit volumes, wherein n is related to prostate volume, PV, as n=PV/(200*v).

In some embodiments, including any of the foregoing, wherein the kit comprises at least n number of unit volumes, wherein TZUL is the BPH transition zone urethra length; and n=TZUL/(11*v).

In some embodiments, including any of the foregoing, wherein the TZUL is from 1.5 cm to 3.5 cm.

In some embodiments, including any of the foregoing, wherein the TZUL is 2 cm, v is 0.05 ml, and n is 3.

In some embodiments, including any of the foregoing, wherein n is 1 to 10.

In some embodiments, including any of the foregoing, wherein n is 1, 2, 3, 4, 5, 6, 7, 8, 9, or 10.

In some embodiments, including any of the foregoing, the needle syringe has a needle slenderness ratio greater than 300.

In some embodiments, including any of the foregoing, the needle syringe has a needle slenderness ratio from 300 to 1200.

In some embodiments, including any of the foregoing, wherein p is from 250 cP to 4000 cP.

In some embodiments, including any of the foregoing, wherein p is from 250 cp to 1100 cP.

In some embodiments, including any of the foregoing, wherein a height of the dosing guide is proportional to a delivery volume in the needle syringe.

In some embodiments, including any of the foregoing, wherein L is from 10 to 20 cm.

In some embodiments, including any of the foregoing, wherein L is from 15 to 20 cm.

In some embodiments, including any of the foregoing, the needle has a slenderness ratio (LID) from 300 to 1200.

In some embodiments, including any of the foregoing, the needle has a slenderness ratio (LID) from 250 to 350.

In some embodiments, including any of the foregoing, wherein the needle has an inner diameter of 0 0.06 cm to 0.08 cm.

In some embodiments, including any of the foregoing, the syringe barrel contains 2 to 10 unit volumes of the composition, excluding dead volume of composition within the syringe barrel.

In some embodiments, including any of the foregoing, wherein:
PV is 20 to 80 cm³,
L is between 15 and 20 cm,
the barrel has a slenderness ratio of between 5 and 20,
D is about 0.06 cm to about 0.08 cm,
v is between 0.05 ml and 0.1 ml, and
μ is between 250 cP and 1100 cP.

In some embodiments, including any of the foregoing, the needle syringe comprises a needle configured for creating multiple lesions along the urethra transition zone.

In some embodiments, including any of the foregoing, the needle syringe comprises up to twenty side holes through which the needle syringe is capable of injecting the composition.

In some embodiments, including any of the foregoing, the needle syringe comprises 10 to 20 side holes through which the needle syringe is capable of injecting the composition.

In some embodiments, including any of the foregoing, the needle syringe comprises 12 side holes through which the needle syringe is capable of injecting the composition.

In some embodiments, including any of the foregoing, the needle syringe is 22 cm long with large 20 Fr/6.7 mm OD.

In some embodiments, including any of the foregoing, the delivery system shaft or sheath is flexible.

In some embodiments, including any of the foregoing, the delivery system or sheath is rigid.

In some embodiments, including any of the foregoing, the barrel comprises a luer port to connect to the needle.

In some embodiments, including any of the foregoing, the guide is configured to be opened for manual retrieval of the needle.

In some embodiments, including any of the foregoing, the extendable needle length for tissue penetration is greater than 5 mm.

In some embodiments, including any of the foregoing, the kit comprises an extendable needle length for tissue penetration is greater about 8 mm.

In some embodiments, set forth herein is a method for treating Benign Prostatic Hyperplasia (BPH) in a subject in need thereof, comprising:
providing the guide of any one of claims 1-29, the delivery device of any one of claims 30-41, or the kit of any one of claims 42-103, and
dispensing a total volume of the composition into a prostate to produce an efficacious outcome for treating BPH, including dispensing no more than a unit volume of the composition into a prostate at a first prostate location using the needle syringe.

In some embodiments, including any of the foregoing, the methods comprise using ultrasound imaging with a transrectal needle; or using a cystoscopic imaging with a transurethral needle.

In some embodiments, including any of the foregoing, the methods comprise the step of placing the needle syringe at a second location within the prostate and dispensing no more than one unit volume of the composition into the prostate at the second location.

In some embodiments, including any of the foregoing, a plurality, n, of the no more than a unit volume, v, are injected into the prostate to treat the prostate at a corresponding n prostate locations, wherein n=TZUL/(11*v).

In some embodiments, including any of the foregoing, a plurality, n, of the no more than a unit volume, v, are injected into the prostate to treat the prostate at a corresponding n prostate locations, wherein n is related to prostate volume, PV, as n=PV/(200*v), wherein the sum of the unit volumes is a total volume for the prostate volume, PV.

In some embodiments, including any of the foregoing, the needle syringe includes a barrel that holds at least n of the unit volumes, wherein n is an integer from 2 to 10; wherein:
n is 2 to 4 when a prostate size is between 20 and 40 grams,
n is 2 to 6 when the prostate size is between 40 and 60 grams,
n is 4 to 8 when the prostate size is between 60 and 80 grams, and
n is 6 to 10 when the prostate size is 80 grams or more.

In some embodiments, including any of the foregoing, a unit volume is 0.05 ml to 0.5 ml, 0.05 ml to 0.1 ml, 0.1 to 0.2 ml, or 0.05 to 0.2 ml.

In some embodiments, including any of the foregoing, the composition has between 4% to 15% vol. of the cytotoxic or cytostatic drug.

In some embodiments, including any of the foregoing, the cytotoxic drug or cytostatic drug is selected from the group consisting of paclitaxel, sirolimus, docetaxel, everolimus, carmustine, mitomycin, tacrolimus, and temsirolimus.

In some embodiments, including any of the foregoing, the cytotoxic drug is docetaxel or paclitaxel.

In some embodiments, including any of the foregoing, the cytostatic drug is sirolimus.

In some embodiments, including any of the foregoing, the method comprises the step of measuring the TZUL and based on the TZUL, selecting a total number of unit volumes, v, of the composition to dispense into the prostate at a respective plurality of locations of the prostate, the sum of the unit volumes being about equal to the total volume.

In some embodiments, including any of the foregoing, the method comprises the step of measuring prostate size and based on the prostate size, selecting a total volume of the composition for treating BPH including selecting the number of unit volumes of the composition to dispense into the prostate at a respective plurality of locations of the prostate, the sum of the unit volumes being about equal to the total volume.

In some embodiments, including any of the foregoing, the prostate size is a prostate volume or a prostrate weight, and wherein the prostate volume or prostate weight is measured by ultrasonic imaging.

In some embodiments, including any of the foregoing, the composition further comprises an alpha blocker or 5-alpha reductase inhibitor or an anti-inflammatory.

In some embodiments, including any of the foregoing, the glycolide-based bioabsorbable copolymer is selected from the set of poly(D,L-lactide-co-glycolide) wherein the molar ratio of lactide to glycolide is 50:50, poly(D,L-lactide-co-glycolide) wherein the molar ratio of lactide to glycolide is 65:35, and poly(D,L-lactide-co-glycolide) wherein the molar ratio of lactide to glycolide is 75:25, and poly(D,L-lactide-co-glycolide) wherein the molar ratio of lactide to glycolide is 85:15.

In some embodiments, including any of the foregoing, the water soluble solvent capable of dissolving the drug and copolymer is selected from the set of N-methylpyrrolidone (NMP) and dimethyl sulfoxide (DMSO), and combinations thereof.

In some embodiments, including any of the foregoing, the glycolide-based bioabsorbable copolymer is selected from the set of poly(D,L-lactide-co-glycolide) (PLGA) and PLGA-PEG-PLGA.

In some embodiments, including any of the foregoing, the glycolide-based bioabsorbable copolymer has a total concentration of 30-55% by wt., the solvent has a total concentration of 69.5-40% by wt., and the cytotoxic or cytostatic drug has a total concentration of 0.5%-30% by wt.; 1%-20% by wt.; or 2%-6% by wt.

In some embodiments, including any of the foregoing, the drug has a release rate of between 5% to 50% during the first 24 hours from injecting the composition into the prostate.

In some embodiments, including any of the foregoing, the solvent comprises N-methyl-pyrrolidone, the drug is paclitaxel and the polymer is PLGA.

In some embodiments, including any of the foregoing, a unit volume is from 0.05 ml to 0.2 ml.

In some embodiments, including any of the foregoing, the plurality of unit volumes is equal to a total volume for treating BPH.

In some embodiments, including any of the foregoing, the composition has between 4% to 15% vol. of the cytotoxic or cytostatic drug, wherein the cytotoxic drug is docetaxel or paclitaxel, and wherein the cytostatic drug is sirolimus.

In some embodiments, including any of the foregoing, the n number of unit volumes of the composition, wherein n is related to TZUL as n=TZUL/(11*v).

In some embodiments, including any of the foregoing, the plurality of unit volumes, n, is related to a prostate volume, PV, for treatment of BPH as n=PV/(200*v).

In some embodiments, including any of the foregoing, the anti-inflammatory is selected from corticosteroids, vasodilators, and combinations thereof.

In some embodiments, including any of the foregoing, the method comprises forcibly pressing the plunger head through the unit volume delivery system until the plunger head has traversed the distance (LDC) for each unit volume.

In some embodiments, including any of the foregoing, the unit volume is as set forth in any one of FIG. 3A, 3B, 14A, or 14B. In some embodiments, including any of the foregoing, the unit volume is 0.05 ml, 0.1 ml, 0.25 ml, 0.5 ml.

In some embodiments, including any of the foregoing, the SRF viscosity is as set forth in any one of FIG. 3A, 3B, 14A, or 14B. In some embodiments, including any of the foregoing, the viscosity (cP) ranges from 267 to 10,000. In some embodiments, including any of the foregoing, the viscosity (cP) ranges from 267 to 1,000.

In some embodiments, including any of the foregoing, the Needle length is as set forth in any one of FIG. 3A, 3B, 14A, or 14B. In some embodiments, including any of the foregoing, the Needle length ranges from 35 cm to 75 cm.

In some embodiments, including any of the foregoing, the Needle ID is as set forth in any one of FIG. 3A, 3B, 14A, or 14B. In some embodiments, including any of the foregoing, the Needle ID ranges from 0.0413 cm to 0.026.

In some embodiments, including any of the foregoing, the bioabsorbable polymer is present at a total concentration set forth in any one of FIG. 2 or 4A. In some embodiments, including any of the foregoing, the bioabsorbable polymer is PLGA8515. In some embodiments, including any of the foregoing, the bioabsorbable polymer is PLGA7525. In some embodiments, including any of the foregoing, the bioabsorbable polymer is PLGA7525. In some embodiments, including any of the foregoing, the bioabsorbable polymer is PLGA5050. In some embodiments, including any of the foregoing, the bioabsorbable polymer is present at a concentration ranging from 35% by weight to 48% by weight. In some embodiments, including any of the foregoing, the bioabsorbable polymer is present at a concentration ranging from 35% by weight to 40% by weight. In some embodiments, including any of the foregoing, the bioabsorbable polymer is present at a concentration ranging from 37% by weight to 40% by weight. In some embodiments, including any of the foregoing, the bioabsorbable polymer is present at a concentration ranging from 37% by weight to 45% by weight. In some embodiments, including any of the foregoing, the bioabsorbable polymer is present at a concentration ranging from 37% by weight to 48% by weight.

In some embodiments, including any of the foregoing, the solvent is present as in any one of FIG. 2 or 4A. In some embodiments, including any of the foregoing, the solvent is NMP.

In some embodiments, including any of the foregoing, the drug is present at a total concentration set forth in any one of FIG. 2 or 4A. In some embodiments, including any of the foregoing, the drug is sirolimus. In some embodiments, including any of the foregoing, the drug is paclitaxel. In some embodiments, including any of the foregoing, the drug is everolimus. In some embodiments, including any of the foregoing, the drug is docetaxel. In some embodiments, including any of the foregoing, the drug is carmustine. In some embodiments, including any of the foregoing, the drug is mitomycin. In some embodiments, including any of the foregoing, the drug is temsirolimus. In some embodiments, including any of the foregoing, the drug is present at a concentration ranging from 3% to 12% by weight.

Embodiments of this disclosure, presented above and in the figures, relate to specific embodiments. A person of skill in the art will appreciate that alternatives can be developed without departing from the spirit and scope of the appended claims herein. Different variants of the embodiments may exist in different forms and it is assumed that other embodiments also all fall within the spirit and scope of appended claims.

1. An apparatus comprising:
a needle syringe containing a composition comprising:
a cytotoxic or cytostatic drug,
a bioabsorbable copolymer, and
a water soluble solvent capable of dissolving the drug and copolymer;
wherein
the composition has an absolute viscosity, μ;
the needle syringe has a needle length L with an inner diameter D; and
the needle syringe comprises a plurality, n, of unit volumes of the composition;
a unit volume, v, is from 0.025 ml to 0.15 ml;
the plurality of unit volumes is equal to a total volume for treating Benign Prostatic Hyperplasia (BPH);
the apparatus has a K-injectable rating (KIR) of between 10 to 300 or 40 to 400; and
KIR is defined as
$$KIR = \frac{μ {L}^{2}}{vD} ( 1 {0}^{- 6} ) .$$

2. The apparatus of claim 1, wherein the cytotoxic drug is docetaxel or paclitaxel.

3. The apparatus of claim 1, wherein the cytostatic drug is sirolimus.

4. The apparatus of claim 1, wherein the composition has between 4% to 15% by volume of the cytotoxic or cytostatic drug, wherein the cytotoxic drug is docetaxel or paclitaxel, and wherein the cytostatic drug is sirolimus.

5. The apparatus of claim 1, wherein the plurality is an integer, n, and wherein n is related to a prostate volume, PV, for treatment of BPH as n=PV/(200*v).

6. The apparatus of claim 1, wherein the apparatus is configured to deliver the composition by way of transurethral, transperineal or transrectal.

7. The apparatus of claim 1, wherein the composition is echogenic and comprises contrast agent to enhance echogenicity.

8. The apparatus of claim 1, wherein the apparatus has a KIR of between 10 and 300.

9. The apparatus of claim 1, wherein the needle syringe comprises a barrel that holds at least n of the unit volumes, n is an integer from 2 to 10; and wherein:
n is from 2 to 4 when a prostate size is between 20 grams and 40 grams,
n is from 2 to 6 when the prostate size is between 40 grams and 60 grams,
n is from 4 to 8 when the prostate size is between 60 grams and 80 grams, and
n is from 6 to 10 when the prostate size is 80 grams or more.

10. The apparatus of claim 1, wherein a unit volume is between 0.05 ml to 0.1 ml.

11. The apparatus of claim 10, wherein a unit volume is between 0.1 ml to 0.2 ml.

12. The apparatus of claim 1, wherein the prostate size is a prostate volume or a prostrate weight, and wherein the prostate volume or prostate weight is determined by an ultrasonic imaging or a magnetic resonance imaging.

13. The apparatus of claim 1, wherein the composition further comprises an alpha blocker or 5-alpha reductase inhibitor or an anti-inflammatory.

14. The apparatus of claim 12, wherein the anti-inflammatory is selected from the group consisting of corticosteroids, vasodilators, and combinations thereof.

15. The apparatus of claim 1, wherein the bioabsorbable copolymer is selected from the group consisting of poly(ethylene glycol) (PEG), poly(D,L-lactide), poly(D,L-lactide-co-glycolide) wherein the molar ratio of lactide to glycolide is 50:50, poly(D,L-lactide-co-glycolide) wherein the molar ratio of lactide to glycolide is 65:35, poly(D,L-lactide-co-glycolide) wherein the molar ratio of lactide to glycolide is 75:25, and poly(D,L-lactide-co-glycolide) wherein the molar ratio of lactide to glycolide is 85:15.

16. The apparatus of claim 1, wherein the water soluble solvent capable of dissolving the drug and copolymer is selected from the group consisting of N-methylpyrrolidone (NMP), dimethyl sulfoxide (DMSO), and combinations thereof.

17. The apparatus of claim 1, wherein the bioabsorbable copolymer is selected from the group consisting of poly(D,L-lactide-co-glycolide) (PLGA) and PLGA-PEG-PLGA.

18. The apparatus of claim 1, wherein the bioabsorbable copolymer has a total concentration of 30-50% by weight, the solvent has a total concentration of 50-30% by weight, and the cytotoxic or cytostatic drug has a total concentration of 0.5%-30% by weight.

19. The apparatus of claim 18, wherein the cytotoxic or cytostatic drug has a total concentration of 2%-6% by weight.

20. The apparatus of claim 1, wherein the solvent comprises N-methyl-pyrrolidone, the drug is paclitaxel, and the bioabsorbable copolymer is PLGA.
